SEQUENCE LISTING (128 sequences)

SEQ ID NO: 1 - DNA, 1551 bp, Bacillus circulans
atggggtggtttttagtgattttacgcaagtggttgattgcttttgtcgcatttttactgatgttctcgtggactggacaacttacgaacaaagcacatgctgcaagcggattttatgtaagcggtaccaaattattggatgctacaggacaaccatttgtgatgcgaggagtcaatcatgcgcacacatggtataaagatcaactatccaccgcaataccagccattgctaaaacaggtgccaacacgatacgtattgtactggcgaatggacacaaatggacgcttgatgatgtaaacaccgtcaacaatattctcaccctctgtgaacaaaacaaactaattgccgttttggaagtacatgacgctacaggaagcgatagtctttccgatttagacaacgccgttaattactggattggtattaaaagcgcgttgatcggcaaggaagaccgtgtaatcattaatatagctaacgagtggtacggaacatgggatggagtcgcctgggctaatggttataagcaagccatacccaaactgcgtaatgctggtctaactcatacgctgattgttgactccgctggatggggacaatatccagattcggtcaaaaattatgggacagaagtactgaatgcagacccgttaaaaaacacagtattctctatccatatgtatgaatatgctgggggcaatgcaagtaccgtcaaatccaatattgacggtgtgctgaacaagaatcttgcactgattatcggcgaatttggtggacaacatacaaacggtgatgtggatgaagccaccattatgagttattcccaagagaagggagtcggctggttggcttggtcctggaagggaaatagcagtgatttggcttatctcgatatgacaaatgattgggctggtaactccctcacctcgttcggtaataccgtagtgaatggcagtaacggcattaaagcaacttctgtgttatccggcatttttggaggtgttacgccaacctcaagccctacttctacacctacatctacgccaacctcaactcctactcctacgccaagtccgaccccgagtccaggtaataacgggacgatcttatatgatttcgaaacaggaactcaaggctggtcgggaaacaatatttcgggaggcccatgggtcaccaatgaatggaaagcaacgggagcgcaaactctcaaagccgatgtctccttacaatccaattccacgcatagtctatatataacctctaatcaaaatctgtctggaaaaagcagtctgaaagcaacggttaagcatgcgaactggggcaatatcggcaacgggatttatgcaaaactatacgtaaagaccgggtccgggtggacatggtacgattccggagagaatctgattcagtcaaacgacggtaccattttgacactatccctcagcggcatttcgaatttgtcctcagtcaaagaaattggggtagaattccgcgcctcctcaaacagtagtggccaatcagctatttatgtagatagtgttagtctgcaatga

SEQ ID NO: 2 - protein, 516 aa, Bacillus circulans
MGWFLVILRKWLIAFVAFLLMFSWTGQLTNKAHAASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGGVTPTSSPTSTPTSTPTSTPTPTPSPTPSPGNNGTILYDFETGTQGWSGNNISGGPWVTNEWKATGAQTLKADVSLQSNSTHSLYITSNQNLSGKSSLKATVKHANWGNIGNGIYAKLYVKTGSGWTWYDSGENLIQSNDGTILTLSLSGISNLSSVKEIGVEFRASSNSSGQSAIYVDSVSLQ

SEQ ID NO: 3 - DNA, 984 bp, Bacillus circulans
atgatgttgatatggatgcagggatggaagtctattctagtcgcgatcttggcgtgtgtgtcagtaggcggtgggcttcctagtccagaagcagccacaggattttatgtaaacggtaccaagctgtatgattcaacgggcaaggcctttgtgatgaggggtgtaaatcatccccacacctggtacaagaatgatctgaacgcggctattccggctatcgcgcaaacgggagccaataccgtacgagtcgtcttgtcgaacgggtcgcaatggaccaaggatgacctgaactccgtcaacagtatcatctcgctggtgtcgcagcatcaaatgatagccgttctggaggtgcatgatgcgacaggcaaagatgagtatgcttcccttgaagcggccgtcgactattggatcagcatcaaaggggcattgatcggaaaagaagaccgcgtcatcgtcaatattgctaatgaatggtatggaaattggaacagcagcggatgggccgatggttataagcaggccattcccaaattaagaaacgcgggcattaagaatacgttgatcgttgatgcagcgggatgggggcaatacccgcaatccatcgtggatgagggggccgcggtatttgcttccgatcaactgaagaatacggtattctccatccatatgtatgagtatgccggtaaggatgccgctacggtgaaaacgaatatggacgatgttttaaacaaaggattgcctttaatcattggggagttcggcggctatcatcaaggtgccgatgtcgatgagattgctattatgaagtacggacagcagaaggaagtgggctggctggcttggtcctggtacggaaacagcccggagctgaacgatttggatctggctgcagggccaagcggaaacctgaccggctggggaaacacggtggttcatggaaccgacgggattcagcaaacctccaagaaagcgggcatttattaa

SEQ ID NO: 4 - protein, 327 aa, Bacillus circulans
MMLIWMQGWKSILVAILACVSVGGGLPSPEAATGFYVNGTKLYDSTGKAFVMRGVNHPHTWYKNDLNAAIPAIAQTGANTVRVVLSNGSQWTKDDLNSVNSIISLVSQHQMIAVLEVHDATGKDEYASLEAAVDYWISIKGALIGKEDRVIVNIANEWYGNWNSSGWADGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY

SEQ ID NO: 5 - DNA, 981 bp, Bacillus circulans
atggccaagttgcaaaagggtacaatcttaacagtcattgcagcactgatgtttgtcattttggggagcgcggcgcccaaagccgcagcagctacaggtttttacgtgaatggaggcaaattgtacgattctacgggtaaaccattttacatgaggggtatcaatcatgggcactcctggtttaaaaatgatttgaacacggctatccctgcgatcgcaaaaacgggtgccaatacggtacgaattgttttatcaaacggtacacaatacaccaaggatgatctgaattccgtaaaaaacatcattaatgtcgtaaatgcaaacaagatgattgctgtgcttgaagtacacgatgccactgggaaagatgacttcaactcgttggatgcagcggtcaactactggataagcatcaaagaagcactgatcgggaaggaagatcgggttattgtaaacattgcaaacgagtggtacggaacatggaacggaagcgcgtgggctgacgggtacaaaaaagctattccgaaattaagagatgcgggtattaaaaataccttgattgtagatgcagcaggctggggtcagtaccctcaatcgatcgtcgattacggacaaagcgtattcgccgcggattcacagaaaaatacggcgttttccattcacatgtatgagtatgcaggcaaggatgcggccaccgtcaaatccaatatggaaaatgtgctgaataaggggctggccttaatcattggtgagttcggaggatatcacaccaatggagatgtcgatgaatatgcaatcatgaaatatggtctggaaaaaggggtaggatggcttgcatggtcttggtacggtaatagctctggattaaactatcttgatttggcaacaggacctaacggcagtttgacgagctatggtaatacggttgtcaatgatacttacggaattaaaaatacgtcccaaaaagcgggaatcttttaa

SEQ ID NO: 6 - protein, 326 aa, Bacillus circulans
MAKLQKGTILTVIAALMFVILGSAAPKAAAATGFYVNGGKLYDSTGKPFYMRGINHGHSWFKNDLNTAIPAIAKTGANTVRIVLSNGTQYTKDDLNSVKNIINVVNANKMIAVLEVHDATGKDDFNSLDAAVNYWISIKEALIGKEDRVIVNIANEWYGTWNGSAWADGYKKAIPKLRDAGIKNTLIVDAAGWGQYPQSIVDYGQSVFAADSQKNTAFSIHMYEYAGKDAATVKSNMENVLNKGLALIIGEFGGYHTNGDVDEYAIMKYGLEKGVGWLAWSWYGNSSGLNYLDLATGPNGSLTSYGNTVVNDTYGIKNTSQKAGIF

SEQ ID NO: 7 - DNA, 984 bp, Paenibacillus polymyxa
atgaaggtattgttaagaaaagcattattgtctggactggtcggcttgctcatcatgattggtttaggaggagttttctccaaggtagaagctgcttcaggattttatgtaagcggtaccaaattgtatgactctacaggcaagccatttgttatgagaggcgtcaatcatgctcacacttggtacaaaaacgatctttatacagctatcccggcaattgcccagacaggtgctaataccgtccgaattgtcctttctaacggaaaccagtacaccaaggatgacattaattccgtgaaaaatattatctctcttgtctccaactataaaatgattgctgtacttgaagttcatgatgctacaggcaaagacgactacgcgtctttggatgcagctgtgaactactggattagcataaaagatgctctgatcggcaaggaagaccgggttatcgtaaacattgcgaacgaatggtatggttcttggaatggaagtggttgggctgatggatacaagcaagcgattcccaagttgagaaacgcaggtatcaaaaatacgctcatcgtcgattgtgccggatggggacagtatcctcagtctatcaatgactttggtaaatctgtatttgcagctgattctttgaagaatacggtattctctattcatatgtatgagttcgctggtaaagatgctcaaaccgttcgaaccaatattgataacgttctgaatcaaggaattcctctgattattggtgaatttggaggttaccaccagggagcagacgtcgacgagacagaaatcatgagatatggccaatccaaaggagtaggctggttagcctggtcctggtatggtaatagttccaacctttcctaccttgatcttgtaacaggacctaatggcaatctgacggattggggaaaaactgtagttaacggaagcaacgggatcaaagaaacatcgaaaaaagctggtatctactaa

SEQ ID NO: 8 - protein, 327 aa, Paenibacillus polymyxa
MKVLLRKALLSGLVGLLIMIGLGGVFSKVEAASGFYVSGTKLYDSTGKPFVMRGVNHAHTWYKNDLYTAIPAIAQTGANTVRIVLSNGNQYTKDDINSVKNIISLVSNYKMIAVLEVHDATGKDDYASLDAAVNYWISIKDALIGKEDRVIVNIANEWYGSWNGSGWADGYKQAIPKLRNAGIKNTLIVDCAGWGQYPQSINDFGKSVFAADSLKNTVFSIHMYEFAGKDAQTVRTNIDNVLNQGIPLIIGEFGGYHQGADVDETEIMRYGQSKGVGWLAWSWYGNSSNLSYLDLVTGPNGNLTDWGKTVVNGSNGIKETSKKAGIY

SEQ ID NO: 9 - DNA, 984 bp, Paenibacillus polymyxa
gtgaacgcattgttaagaaaagcattattgtctggactcgctggtctgcttatcatgattggtttggggggattcttctccaaggcgcaagctgcttcaggattttatgtaagcggtaccaatctgtatgactctacaggcaaaccgttcgttatgagaggcgtcaatcatgctcacacttggtacaaaaacgatctttatactgctatcccagcaattgctaaaacaggtgctaatacagtccgaattgtcctttctaacggaaaccagtacaccaaggatgacattaattccgtgaaaaatattatctctctcgtctccaaccataaaatgattgctgtacttgaagttcatgacgctacaggtaaagacgactatgcgtctttggatgcagcagtgaattactggattagtataaaagatgctctgatcggcaaggaagatcgggttatcgtgaacattgcgaacgaatggtatggctcttggaatggaggcggttgggcagatgggtataagcaagcgattcccaagctgagaaacgcaggcatcaaaaatacgctcatcgtcgattgtgctggatggggacaataccctcagtctatcaatgactttggtaaatctgtgtttgcagctgattctttgaaaaataccgttttctccattcatatgtatgaatttgctggcaaagatgttcaaacggttcgaaccaatattgataacgttctgtatcaagggctccctttgattattggtgaatttggcggttaccatcagggagcagacgtcgacgagacagaaatcatgagatacggccaatctaaaagcgtaggctggttagcctggtcctggtatggcaatagctccaaccttaattatcttgatcttgtgacaggacctaacggcaatctgaccgattggggtcgcaccgtggtagagggagccaacgggatcaaagaaacatcgaaaaaagcgggtatcttctaa

SEQ ID NO: 10 - protein, 327 aa, Paenibacillus polymyxa
MNALLRKALLSGLAGLLIMIGLGGFFSKAQAASGFYVSGTNLYDSTGKPFVMRGVNHAHTWYKNDLYTAIPAIAKTGANTVRIVLSNGNQYTKDDINSVKNIISLVSNHKMIAVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLYQGLPLIIGEFGGYHQGADVDETEIMRYGQSKSVGWLAWSWYGNSSNLNYLDLVTGPNGNLTDWGRTVVEGANGIKETSKKAGIF

SEQ ID NO: 11 - DNA, 960 bp, Paenibacillus sp. A1
atgaaatacctgctgccgaccgctgctgctggtctgctgctcctcgctgcccagccggcgatggccatggctacaggtttttatgtaagcggtaacaagttatacgattccactggcaagccttttgttatgagaggtgttaatcacggacattcctggttcaaaaatgatttgaataccgctatccctgccatcgccaaaacaggtgccaatacggtacgcattgttctttcgaatggtagcctgtacaccaaagatgatctgaacgctgttaaaaatattattaatgtggttaaccagaataaaatgatagctgtactcgaagtacatgacgccacagggaaagatgactataattcgttggatgcggcggtgaactactggattagtattaaggaagctttgattggaaaagaagatcgggtaattgtcaacatcgccaatgaatggtatggaacgtggaatggaagtgcgtgggctgatggttacaaaaaagccattccgaaactccgaaatgcaggaattaaaaatacgctaattgtggatgcagccggatggggacagttccctcaatccatcgtggattatggacaaagtgtatttgcagccgattcacagaaaaataccgtcttctccattcatatgtatgagtatgctggcaaagatgctgcaacggtcaaagccaatatggagaatgtgctgaacaaaggattggctctgatcattggtgaattcgggggatatcacacaaacggtgatgtggatgagtatgccatcatgagatatggtcaggaaaaaggggtaggctggcttgcctggtcttggtacggaaacagctccggtttgaactatctggacatggccacaggtccgaacggaagcttaacgagttttggcaacactgttgttaatgatacctatggtattaaaaacacttcccaaaaagcggggattttctaa

SEQ ID NO: 12 - protein, 319 aa, Paenibacillus sp. A1
MKYLLPTAAAGLLLLAAQPAMAMATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 13 - DNA, 984 bp, Paenibacillus sp. CH-3
atgagacaacttttagcaaaaggtattttagctgcactggtcatgatgttagcgatgtatggattggggaatctctcttctaaagcttcggctgcaacaggtttttatgtaagcggtaccactctatatgattctactggtaaaccttttgtaatgcgcggtgtcaatcattcgcatacctggttcaaaaatgatctaaatgcagccatccctgctattgccaaaacaggtgcaaatacagtacgtatcgttttatctaatggtgttcagtatactagagatgatgtaaactcagtcaaaaatattatttccctggttaaccaaaacaaaatgattgctgttcttgaggtgcatgatgctaccggtaaagacgattacgcttctcttgatgccgctgtaaactactggatcagcatcaaagatgccttgattggcaaggaagatcgagtcattgttaatattgccaatgaatggtacggtacatggaatggcagtgcttgggcagatggttataagcaggctattcccaaactaagaaatgcaggcatcaaaaacactttaatcgttgatgccgccggctggggacaatgtcctcaatcgatcgttgattacgggcaaagtgtatttgcagcagattcgcttaaaaatacaattttctctattcacatgtatgaatatgcaggcggtacagatgcgatcgtcaaaagcaatatggaaaatgtactgaacaaaggacttcctttgatcatcggtgaatttggcgggcagcatacaaacggcgatgtagatgaacatgcaattatgcgttatggtcagcaaaaaggtgtaggttggctggcatggtcgtggtatggcaacaatagtgaactcagttatctggatttggctacaggtcccgccggtagtctgacaagtatcggcaatacgattgtaaatgatccatatggtatcaaagctacctcgaaaaaagcgggtatcttctaa

SEQ ID NO: 14 - protein, 327 aa, Paenibacillus sp. CH-3
MRQLLAKGILAALVMMLAMYGLGNLSSKASAATGFYVSGTTLYDSTGKPFVMRGVNHSHTWFKNDLNAAIPAIAKTGANTVRIVLSNGVQYTRDDVNSVKNIISLVNQNKMIAVLEVHDATGKDDYASLDAAVNYWISIKDALIGKEDRVIVNIANEWYGTWNGSAWADGYKQAIPKLRNAGIKNTLIVDAAGWGQCPQSIVDYGQSVFAADSLKNTIFSIHMYEYAGGTDAIVKSNMENVLNKGLPLIIGEFGGQHTNGDVDEHAIMRYGQQKGVGWLAWSWYGNNSELSYLDLATGPAGSLTSIGNTIVNDPYGIKATSKKAGIF

SEQ ID NO: 15 - DNA, 978 bp, Paenibacillus amylolyticus
atggttaatctgaaaaagtgtacaatcttcacggttattgctacactcatgttcatggtattagggagtgcagcacccaaagcatctgctgctacaggattttatgtaagcggtaacaagttatacgattccacaggcaaggcttttgtcatgagaggtgttaatcacggacattcctggttcaaaaatgatttgaataccgctatccctgcaatcgccaaaacaggtgccaatacggtacgcattgttctttcgaatggtagcctgtacaccaaagatgatctgaacgctgttaaaaatattattaatgtggttaaccaaaataaaatgatagctgtactcgaggtgcatgacgccacagggaaagatgactataattcgttggatgcggcagtgaactactggattagcattaaggaagctttgattggcaaagaagatcgggtcatcgtcaatatcgccaatgaatggtatggaacgtggaatggaagtgcgtgggctgatggttacaaaaaagccattccgaaactccgaaatgcgggaattaaaaatacgctaattgtggatgcagccggatggggacagttccctcaatccatcgtggattatggacaaagtgtatttgcaaccgattctcagaaaaatacggtcttctccattcatatgtatgagtatgctggcaaagatgctgcaaccgtcaaagccaatatggaaaatgtgctgaacaaaggattggctctgatcattggtgagttcgggggataccacacaaacggtgatgtggacgagtatgccatcatgagatatggtcaggaaaaaggggtgggctggctggcctggtcctggtatggaaacagttctggtctgaactacctggacatggctacaggtccgaacggaagtttgacgagcttcggaaacaccgtagtgaatgatacctatggaattaaaaaaacttctcaaaaagcggggattttc

SEQ ID NO: 16 - protein, 326 aa, Paenibacillus amylolyticus
MVNLKKCTIFTVIATLMFMVLGSAAPKASAATGFYVSGNKLYDSTGKAFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFATDSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKKTSQKAGIF

SEQ ID NO: 17 - protein, 296 aa, Paenibacillus amylolyticus
ATGFYVSGNKLYDSTGKAFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFATDSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKKTSQKAGIF

SEQ ID NO: 18 - DNA, 978 bp, Paenibacillus pabuli
atggtcaagttgcaaaagggtacgatcatcaccgtcattgctgcgctcattttggttatgttgggaagtgctgcacccaaagcttctgctgctgctggtttttatgtaagcggtaacaagttgtatgactctacgggtaaagcttttgtcatgcggggcgtcaaccacagtcatacctggttcaagaacgatctaaacacagcgatacccgccattgcaaaaacaggtgcgaacacggtacgtattgtgctctccaatgggacgcaatataccaaagatgatttgaacgccgttaaaaacataatcaacctggtgagtcagaacaaaatgatcgcagtgctcgaagtacatgatgcaactggtaaagatgactacaattcgttggatgcagcagtcaactactggattagcatcaaggaagctctgattggcaaggaagaccgcgttatcgtcaatattgccaatgaatggtacgggacctggaacggcagtgcctgggctgacgggtacaaaaaagcaattccgaaactgagaaatgccggcattaaaaatacattaattgtagatgcagctggctggggccaatatccgcaatctattgtggactatggtcaaagtgtttttgcagcagatgcccagaaaaatacggttttctccattcacatgtatgaatatgcaggtaaagatgccgcaacggtcaaagccaacatggaaaacgtgctgaacaaaggtttggccctgatcatcggtgagtttggtggataccacaccaatggggacgtcgatgaatatgcaatcatgaaatacggtcaggaaaaaggagtaggctggctcgcatggtcctggtatgggaacaactccgatctcaattatctggatttggctacaggtccaaacggaactttaacaagctttggcaacacggtggtttatgacacgtatggaattaaaaacacttcggtaaaagcagggatctat

SEQ ID NO: 19 - protein, 326 aa, Paenibacillus pabuli
MVKLQKGTIITVIAALILVMLGSAAPKASAAAGFYVSGNKLYDSTGKAFVMRGVNHSHTWFKNDLNTAIPAIAKTGANTVRIVLSNGTQYTKDDLNAVKNIINLVSQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADAQKNTVFSIHMYEYAGKDAATVKANMENVLNKGLALIIGEFGGYHTNGDVDEYAIMKYGQEKGVGWLAWSWYGNNSDLNYLDLATGPNGTLTSFGNTVVYDTYGIKNTSVKAGIY

SEQ ID NO: 20 - DNA, 945 bp, Paenibacillus hunanensis
gtgtttatgttagcgatgtatggatgggctggactgactggtcaagcttcagctgctacaggtttttatgtaagcggtaccaaattatacgactctacaggcaagccatttgtgatgcgtggtgtgaatcattcccacacctggttcaaaaatgacctgaatgcagcgatccctgcaattgccaaaacaggcgccaacacggtacgtatcgtattatcgaatggcgtgcagtacaccagagatgatgtaaactccgtcaaaaatatcatctctctcgtcaaccagaacaaaatgatcgcagtactggaggttcatgatgcaacaggcaaggacgattacgcttcgctcgatgccgcaatcaactactggatcagcatcaaggatgcgctgatcggtaaagaggatcgcgttatcgtcaatattgccaacgaatggtatggcacatggaatggaagcgcatgggcagatggctacaaacaggcgattccaaagctccgtaatgcgggtataaaaaatacgctgattgttgacgcagccggctggggtcaatatccacaatcgatcgttgattatggacaaagtgtatttgcagcggattcgttaaaaaatacggttttctcgatccatatgtatgagtatgcaggtggaaccgatgcgatggtcaaagccaacatggagggcgtactcaataaaggtctgccactgatcattggtgaatttggcggacagcacacaaatggagacgtggatgagctggcgatcatgcgttacggacaacaaaaaggagtaggctggctcgcctggtcctggtacggcaacaatagtgatctgagttatctcgatctagcgacaggtccaaatggtagcctgaccacgtttggtaatacggtggtaaatgacaccaacggtatcaaagccacctccaaaaaagcaggtattttccag

SEQ ID NO: 21 - protein, 315 aa, Paenibacillus hunanensis
MFMLAMYGWAGLTGQASAATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 22 - DNA, 978 bp, Paenibacillus tundrae
atggtcaagttgcaaaagtgtacagtctttaccgtaattgctgcacttatgttggtgattctggcgagtgctgcacccaaagcgtctgctgctacaggattttatgtaagcggaggcaaattgtacgattctactggcaaggcatttgttatgagaggtgtcaatcatggacattcatggtttaagaacgacttgaacacggctattcctgcgatagccaaaacaggtgccaacaccgtacggattgtgctctccaatggcgtacagtacaccaaagacgatctgaactctgttaaaaacatcattaatgttgtaagcgtaaacaaaatgattgcggtgctcgaagtacatgatgcaacaggtaaggatgactataattcgttggatgcagcggtgaactactggattagcatcaaggaagcactcattggcaaagaagacagagttatcgtaaatatcgcgaacgaatggtatggaacatggaacggcagtgcctgggctgacggatacaaaaaagcaattccgaagctgagaaatgccggtattaaaaatacattgatcgtggatgcagcgggctgggggcagtacccgcaatccatcgtggattatggacaaagtgtatttgcagcggattcacagaaaaacaccgtattctcgattcacatgtatgaatatgccggtaaagacgcagcaaccgtaaaagccaacatggaaagcgtattaaacaaaggtctggccctgatcatcggtgaattcggtggatatcacacgaacggggatgtcgatgaatatgcgatcatgaaatatggtcaggaaaaaggggtaggctggctcgcatggtcctggtatggcaatagctccgatttgaactatttggacttggctacgggacctaacggaagtttgactagctttggaaacacagtcgtcaacgacacttatggaatcaaaaatacttcaaaaaaagcagggatctac

SEQ ID NO: 23 - protein, 326 aa, Paenibacillus tundrae
MVKLQKCTVFTVIAALMLVILASAAPKASAATGFYVSGGKLYDSTGKAFVMRGVNHGHSWFKNDLNTAIPAIAKTGANTVRIVLSNGVQYTKDDLNSVKNIINVVSVNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADSQKNTVFSIHMYEYAGKDAATVKANMESVLNKGLALIIGEFGGYHTNGDVDEYAIMKYGQEKGVGWLAWSWYGNSSDLNYLDLATGPNGSLTSFGNTVVNDTYGIKNTSKKAGIY

SEQ ID NO: 24 - protein, 296 aa, Paenibacillus tundrae
ATGFYVSGGKLYDSTGKAFVMRGVNHGHSWFKNDLNTAIPAIAKTGANTVRIVLSNGVQYTKDDLNSVKNIINVVSVNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADSQKNTVFSIHMYEYAGKDAATVKANMESVLNKGLALIIGEFGGYHTNGDVDEYAIMKYGQEKGVGWLAWSWYGNSSDLNYLDLATGPNGSLTSFGNTVVNDTYGIKNTSKKAGIY

SEQ ID NO: 25 - DNA, 1542 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaagcggcttttatgtttcaggcacaaaactgctggatgcaacaggccaaccgtttgttatgagaggcgttaatcatgcacatacgtggtataaagatcaactgtcaacagcaattccggcaatcgcaaaaacaggcgcaaatacaattagaattgttctggcgaatggccataaatggacactggatgatgttaacacagtcaacaatattctgacactgtgcgaacagaataaactgattgcagttctggaagttcatgatgcgacaggctcagattcactgtcagatctggataatgcagtcaattattggatcggcattaaatcagcactgatcggcaaagaagatcgcgtcattattaacattgcgaacgaatggtatggcacatgggatggcgttgcatgggcaaatggctataaacaagcgattccgaaactgagaaatgcaggcctgacacatacactgattgttgattcagcaggctggggacaatatccggattcagttaaaaactatggcacagaagttctgaacgcagatccgctgaaaaatacagtctttagcatccacatgtacgaatatgcaggcggaaatgcatcaacagtgaaatcaaatattgatggcgtcctgaataaaaacctggcactgattattggcgaatttggcggacaacatacaaatggcgacgttgatgaagcaacgattatgtcatatagccaagaaaaaggcgttggctggcttgcatggtcatggaaaggcaattcatcagatcttgcatatctggatatgacgaatgattgggcaggcaatagcctgacatcatttggcaatacagttgtcaatggcagcaatggcattaaagcaacatcagttctgtcaggcatttttggcggagttacaccgacatcatcaccgacaagcacaccgacgtcaacacctacatcaacgccgacaccgacacctagcccgacaccttcaccgggaaataatggcacaattctgtatgattttgaaacaggcacacaaggctggtcaggcaataacatttcaggcggaccgtgggttacaaatgaatggaaagcgacaggcgcacaaacactgaaagcagatgtttcacttcaaagcaattcaacgcatagcctgtatatcacaagcaatcaaaatctgagcggcaaatcaagcctgaaagcaacagttaaacatgcgaattggggcaatattggcaatggaatttatgcgaaactgtacgttaaaacaggcagcggctggacatggtatgattcaggcgaaaatctgattcagtcaaacgatggaacaatcctgacactttcactttcaggcattagcaatctgagcagcgttaaagaaattggcgtcgaatttagagcaagctcaaatagctcaggccaaagcgcaatttatgttgatagcgtttcactgcag

SEQ ID NO: 26 - protein, 514 aa, Artificial Sequence
  precursor protein expressed from synthetic construct
MRSKKLWISLLFALTLIFTMAFSNMSAQAAGKASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGGVTPTSSPTSTPTSTPTSTPTPTPSPTPSPGNNGTILYDFETGTQGWSGNNISGGPWVTNEWKATGAQTLKADVSLQSNSTHSLYITSNQNLSGKSSLKATVKHANWGNIGNGIYAKLYVKTGSGWTWYDSGENLIQSNDGTILTLSLSGISNLSSVKEIGVEFRASSNSSGQSAIYVDSVSLQ

SEQ ID NO: 27 - protein, 485 aa, Artificial Sequence
  mature protein expressed from synthetic construct
AGKASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGGVTPTSSPTSTPTSTPTSTPTPTPSPTPSPGNNGTILYDFETGTQGWSGNNISGGPWVTNEWKATGAQTLKADVSLQSNSTHSLYITSNQNLSGKSSLKATVKHANWGNIGNGIYAKLYVKTGSGWTWYDSGENLIQSNDGTILTLSLSGISNLSSVKEIGVEFRASSNSSGQSAIYVDSVSLQ

SEQ ID NO: 28 - protein, 482 aa, Artificial Sequence
  mature protein sequence, based on the predicted cleavage of the naturally occurring sequence
ASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGGVTPTSSPTSTPTSTPTSTPTPTPSPTPSPGNNGTILYDFETGTQGWSGNNISGGPWVTNEWKATGAQTLKADVSLQSNSTHSLYITSNQNLSGKSSLKATVKHANWGNIGNGIYAKLYVKTGSGWTWYDSGENLIQSNDGTILTLSLSGISNLSSVKEIGVEFRASSNSSGQSAIYVDSVSLQ

SEQ ID NO: 29 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaacaggcttttatgtcaatggcacgaaactgtatgatagcacaggcaaagcatttgttatgagaggcgttaatcatccgcatacgtggtataaaaacgatctgaatgcagcaattccggctattgcacaaacaggcgcaaatacagttagagttgttctgtcaaatggcagccaatggacaaaagatgatctgaatagcgtcaacagcattatttcactggttagccaacatcaaatgattgcagttctggaagttcatgatgcaacgggcaaagatgaatatgcatcactggaagcagcagtcgattattggatttcaattaaaggcgcactgatcggcaaagaagatagagtcattgtcaatattgcgaacgaatggtatggcaattggaattcatcaggctgggcagatggctataaacaagcgattccgaaactgagaaatgcaggcattaaaaacacactgattgttgatgcagcaggctggggacaatatccgcaatcaattgtcgatgaaggcgcagcagtttttgcatcagatcaactgaaaaacacggtctttagcatccacatgtatgaatacgctggaaaagatgcagcaacagtcaaaacaaatatggatgacgttctgaataaaggcctgccgctgattattggcgaatttggcggatatcatcaaggcgcagatgttgatgaaattgcgattatgaaatacggccagcaaaaagaggttggctggcttgcatggtcatggtatggaaactcaccggaactgaatgatctggatctggcagcaggaccgtcaggcaatctgacaggatggggcaatacagttgttcatggcacagatggcattcaacagacatcaaaaaaagcaggcatctat

SEQ ID NO: 30 - protein, 328 aa, Artificial Sequence
  precursor protein expressed from synthetic construct
MRSKKLWISLLFALTLIFTMAFSNMSAQAAGKATGFYVNGTKLYDSTGKAFVMRGVNHPHTWYKNDLNAAIPAIAQTGANTVRVVLSNGSQWTKDDLNSVNSIISLVSQHQMIAVLEVHDATGKDEYASLEAAVDYWISIKGALIGKEDRVIVNIANEWYGNWNSSGWADGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY

SEQ ID NO: 31 - protein, 299 aa, Artificial Sequence
  mature protein expressed from synthetic construct
AGKATGFYVNGTKLYDSTGKAFVMRGVNHPHTWYKNDLNAAIPAIAQTGANTVRVVLSNGSQWTKDDLNSVNSIISLVSQHQMIAVLEVHDATGKDEYASLEAAVDYWISIKGALIGKEDRVIVNIANEWYGNWNSSGWADGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY

SEQ ID NO: 32 - protein, 296 aa, Artificial Sequence
  mature protein sequence, based on predicted cleavage of naturally occurring sequence
ATGFYVNGTKLYDSTGKAFVMRGVNHPHTWYKNDLNAAIPAIAQTGANTVRVVLSNGSQWTKDDLNSVNSIISLVSQHQMIAVLEVHDATGKDEYASLEAAVDYWISIKGALIGKEDRVIVNIANEWYGNWNSSGWADGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY

SEQ ID NO: 33 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaacaggcttttatgttaatggcggaaaactgtatgatagcacaggcaaaccgttttatatgcgtggcattaatcatggccatagctggtttaaaaacgatctgaatacagcgattccggctattgcaaaaacaggcgcaaatacagttagaattgttctgtcaaatggcacgcagtatacgaaagatgatctgaactcagtcaaaaacatcatcaatgtcgtcaacgcgaacaaaatgattgcagttctggaagttcatgatgcaacgggcaaagatgatttcaattcactggatgcagcagtcaactattggatctcaattaaagaagcgctgatcggcaaagaagatcgcgttattgttaatattgcgaacgaatggtatggcacatggaatggctcagcatgggcagatggctacaaaaaagcaattccgaaactgagagatgcaggcattaaaaacacactgattgttgatgcggcaggctggggacaatatccgcaatcaattgttgattatggccaaagcgtttttgcagcagatagccagaaaaatacagcgtttagcatccacatgtatgaatatgcgggaaaagatgcagcaacagtcaaaagcaatatggaaaacgtcctgaataaaggcctggcactgattattggcgaatttggcggatatcatacaaatggcgacgttgacgaatatgcgattatgaaatatggcctggaaaaaggcgttggctggcttgcatggtcatggtatggaaattcatcaggccttaattatctggatctggcaacaggaccgaatggcagcctgacatcatatggcaatacagttgtcaatgatacgtatggcatcaaaaatacgtcacagaaagcaggcatcttt

SEQ ID NO: 34 - protein, 328 aa, Artificial Sequence
  precursor protein expressed from synthetic construct
MRSKKLWISLLFALTLIFTMAFSNMSAQAAGKATGFYVNGGKLYDSTGKPFYMRGINHGHSWFKNDLNTAIPAIAKTGANTVRIVLSNGTQYTKDDLNSVKNIINVVNANKMIAVLEVHDATGKDDFNSLDAAVNYWISIKEALIGKEDRVIVNIANEWYGTWNGSAWADGYKKAIPKLRDAGIKNTLIVDAAGWGQYPQSIVDYGQSVFAADSQKNTAFSIHMYEYAGKDAATVKSNMENVLNKGLALIIGEFGGYHTNGDVDEYAIMKYGLEKGVGWLAWSWYGNSSGLNYLDLATGPNGSLTSYGNTVVNDTYGIKNTSQKAGIF

SEQ ID NO: 35 - protein, 299 aa, Artificial Sequence
  mature protein expressed from synthetic construct
AGKATGFYVNGGKLYDSTGKPFYMRGINHGHSWFKNDLNTAIPAIAKTGANTVRIVLSNGTQYTKDDLNSVKNIINVVNANKMIAVLEVHDATGKDDFNSLDAAVNYWISIKEALIGKEDRVIVNIANEWYGTWNGSAWADGYKKAIPKLRDAGIKNTLIVDAAGWGQYPQSIVDYGQSVFAADSQKNTAFSIHMYEYAGKDAATVKSNMENVLNKGLALIIGEFGGYHTNGDVDEYAIMKYGLEKGVGWLAWSWYGNSSGLNYLDLATGPNGSLTSYGNTVVNDTYGIKNTSQKAGIF

SEQ ID NO: 36 - protein, 296 aa, Artificial Sequence
  mature protein sequence, based on predicted cleavage of naturally occurring protein sequence
ATGFYVNGGKLYDSTGKPFYMRGINHGHSWFKNDLNTAIPAIAKTGANTVRIVLSNGTQYTKDDLNSVKNIINVVNANKMIAVLEVHDATGKDDFNSLDAAVNYWISIKEALIGKEDRVIVNIANEWYGTWNGSAWADGYKKAIPKLRDAGIKNTLIVDAAGWGQYPQSIVDYGQSVFAADSQKNTAFSIHMYEYAGKDAATVKSNMENVLNKGLALIIGEFGGYHTNGDVDEYAIMKYGLEKGVGWLAWSWYGNSSGLNYLDLATGPNGSLTSYGNTVVNDTYGIKNTSQKAGIF

SEQ ID NO: 37 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcagcaggcttttatgtttcaggcaacaagctgtatgattcaacaggaaaagcatttgttatgagaggcgttaatcattcacatacatggtttaagaacgatcttaatacagccattccggcaatcgcgaagacaggagcaaatacagtgagaattgttctttcaaacggaacgcaatatacaaaagatgacctgaacgccgttaagaatatcattaatctggtttcacaaaataagatgattgcagttctggaggttcatgatgcaacaggcaaggatgactacaatagcctggatgcagcggtcaattactggatttcaattaaagaagcacttattggcaaagaggatagagttattgttaatatcgcaaatgaatggtatggaacgtggaacggctcagcatgggcagatggctacaaaaaagcaattccgaaactgagaaatgcaggaatcaaaaatacactgattgttgacgccgcaggctggggacaatatccgcaaagcatcgttgattatggccaaagcgtttttgccgcagacgcacagaaaaacacggttttctcaattcatatgtacgagtatgctggaaaggatgctgcaacggttaaagctaacatggaaaatgttctgaataaaggcctggcactgatcattggcgaatttggaggctatcacacaaatggcgatgttgatgaatacgcaattatgaaatatggacaagaaaaaggcgttggatggcttgcatggtcatggtacggaaacaactcagaccttaattacctggacctggctacgggaccgaatggcacactgacatcattcggcaatacggtcgtttatgacacgtatggcatcaagaacacgagcgtgaaagccggcatttat

SEQ ID NO: 38 - protein, 328 aa, Artificial Sequence
  precursor protein expressed from synthetic construct
MRSKKLWISLLFALTLIFTMAFSNMSAQAAGKAAGFYVSGNKLYDSTGKAFVMRGVNHSHTWFKNDLNTAIPAIAKTGANTVRIVLSNGTQYTKDDLNAVKNIINLVSQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADAQKNTVFSIHMYEYAGKDAATVKANMENVLNKGLALIIGEFGGYHTNGDVDEYAIMKYGQEKGVGWLAWSWYGNNSDLNYLDLATGPNGTLTSFGNTVVYDTYGIKNTSVKAGIY

SEQ ID NO: 39 - protein, 299 aa, Artificial Sequence
  mature protein expressed from synthetic construct
AGKAAGFYVSGNKLYDSTGKAFVMRGVNHSHTWFKNDLNTAIPAIAKTGANTVRIVLSNGTQYTKDDLNAVKNIINLVSQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADAQKNTVFSIHMYEYAGKDAATVKANMENVLNKGLALIIGEFGGYHTNGDVDEYAIMKYGQEKGVGWLAWSWYGNNSDLNYLDLATGPNGTLTSFGNTVVYDTYGIKNTSVKAGIY

SEQ ID NO: 40 - protein, 296 aa, Artificial Sequence
  mature protein sequence, based on the predicted cleavage of the naturally occurring sequence
AAGFYVSGNKLYDSTGKAFVMRGVNHSHTWFKNDLNTAIPAIAKTGANTVRIVLSNGTQYTKDDLNAVKNIINLVSQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFAADAQKNTVFSIHMYEYAGKDAATVKANMENVLNKGLALIIGEFGGYHTNGDVDEYAIMKYGQEKGVGWLAWSWYGNNSDLNYLDLATGPNGTLTSFGNTVVYDTYGIKNTSVKAGIY

SEQ ID NO: 41 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaagcggcttttatgtttcaggcacaaaactgtatgatagcacaggcaaaccgtttgttatgagaggcgttaatcatgcacatacgtggtataaaaacgatctgtatacggcaattccggctattgcacaaacaggcgcaaatacagttagaattgttctgagcaatggcaaccagtatacgaaagatgatatcaacagcgtcaaaaacattatcagcctggtcagcaactataaaatgattgcagttctggaagtccatgatgcaacgggcaaagatgattatgcatcactggatgcagcagtcaattattggattagcattaaagatgcgctgatcggcaaagaagatcgcgttattgttaatattgcgaacgaatggtatggctcatggaatggctcaggctgggcagatggctataaacaagcaattccgaaactgagaaatgcaggcattaaaaacacactgattgttgattgcgcaggctggggacaatatccgcaatcaattaatgattttggcaaaagcgtttttgcagcggatagcctgaaaaatacagtctttagcatccatatgtatgaatttgcgggaaaagatgcacagacagtccgcacaaatattgataatgtcctgaatcaaggcatcccgctgattattggcgaatttggcggatatcatcaaggcgcagatgttgatgaaacagaaattatgagatacggccaatcaaaaggcgttggctggcttgcatggtcatggtatggaaattcaagcaatctgtcatatctggatctggttacaggaccgaatggcaatcttacagattggggcaaaacagttgttaatggctcaaatggcatcaaagaaacgtcaaaaaaagcaggcatctat

SEQ ID NO: 42 - protein, 328 aa, Artificial Sequence
  precursor protein expressed from synthetic construct
MRSKKLWISLLFALTLIFTMAFSNMSAQAAGKASGFYVSGTKLYDSTGKPFVMRGVNHAHTWYKNDLYTAIPAIAQTGANTVRIVLSNGNQYTKDDINSVKNIISLVSNYKMIAVLEVHDATGKDDYASLDAAVNYWISIKDALIGKEDRVIVNIANEWYGSWNGSGWADGYKQAIPKLRNAGIKNTLIVDCAGWGQYPQSINDFGKSVFAADSLKNTVFSIHMYEFAGKDAQTVRTNIDNVLNQGIPLIIGEFGGYHQGADVDETEIMRYGQSKGVGWLAWSWYGNSSNLSYLDLVTGPNGNLTDWGKTVVNGSNGIKETSKKAGIY

SEQ ID NO: 43 - protein, 299 aa, Artificial Sequence
  mature protein expressed from synthetic construct
AGKASGFYVSGTKLYDSTGKPFVMRGVNHAHTWYKNDLYTAIPAIAQTGANTVRIVLSNGNQYTKDDINSVKNIISLVSNYKMIAVLEVHDATGKDDYASLDAAVNYWISIKDALIGKEDRVIVNIANEWYGSWNGSGWADGYKQAIPKLRNAGIKNTLIVDCAGWGQYPQSINDFGKSVFAADSLKNTVFSIHMYEFAGKDAQTVRTNIDNVLNQGIPLIIGEFGGYHQGADVDETEIMRYGQSKGVGWLAWSWYGNSSNLSYLDLVTGPNGNLTDWGKTVVNGSNGIKETSKKAGIY

SEQ ID NO: 44 - protein, 296 aa, Artificial Sequence
  mature protein sequence, based on the predicted cleavage of the naturally occurring sequence
ASGFYVSGTKLYDSTGKPFVMRGVNHAHTWYKNDLYTAIPAIAQTGANTVRIVLSNGNQYTKDDINSVKNIISLVSNYKMIAVLEVHDATGKDDYASLDAAVNYWISIKDALIGKEDRVIVNIANEWYGSWNGSGWADGYKQAIPKLRNAGIKNTLIVDCAGWGQYPQSINDFGKSVFAADSLKNTVFSIHMYEFAGKDAQTVRTNIDNVLNQGIPLIIGEFGGYHQGADVDETEIMRYGQSKGVGWLAWSWYGNSSNLSYLDLVTGPNGNLTDWGKTVVNGSNGIKETSKKAGIY

SEQ ID NO: 45 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaagcggcttttatgtttcaggcacaaatctgtatgatagcacaggcaaaccgtttgttatgagaggcgttaatcatgcacatacgtggtataaaaacgatctgtatacggcaattccggcaatcgcaaaaacaggcgcaaatacagttagaattgttctgagcaatggcaaccagtatacgaaagatgatatcaacagcgtcaaaaacattatcagcctggtcagcaaccataaaatgattgcagttctggaagttcatgatgcaacgggcaaagatgattatgcatcactggatgcagcagtcaattattggattagcattaaagatgcgctgatcggcaaagaagatcgcgttattgttaatattgcgaacgaatggtatggctcatggaatggcggaggctgggcagatggctataaacaagcaattccgaaactgagaaatgcaggcattaaaaacacactgattgttgattgcgcaggctggggacaatatccgcaatcaattaatgattttggcaaaagcgtttttgcagcggatagcctgaaaaatacagtctttagcatccatatgtatgaatttgcaggcaaagacgtccaaacagtccgcacaaatattgataatgtcctgtatcaaggcctgccgctgattattggcgaatttggcggatatcatcaaggcgcagatgttgatgaaacagaaattatgagatacggccagtcaaaatcagttggctggcttgcatggtcatggtatggaaattcaagcaatctgaactatctggatctggttacaggaccgaatggcaatcttacagattggggcagaacagttgttgaaggcgctaatggaattaaagaaacgtcaaaaaaagcaggcattttt

SEQ ID NO: 46 - protein, 328 aa, Artificial Sequence
  precursor protein expressed from synthetic construct
MRSKKLWISLLFALTLIFTMAFSNMSAQAAGKASGFYVSGTNLYDSTGKPFVMRGVNHAHTWYKNDLYTAIPAIAKTGANTVRIVLSNGNQYTKDDINSVKNIISLVSNHKMIAVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLYQGLPLIIGEFGGYHQGADVDETEIMRYGQSKSVGWLAWSWYGNSSNLNYLDLVTGPNGNLTDWGRTVVEGANGIKETSKKAGIF

SEQ ID NO: 47 - protein, 299 aa, Artificial Sequence
  mature protein expressed from synthetic construct
AGKASGFYVSGTNLYDSTGKPFVMRGVNHAHTWYKNDLYTAIPAIAKTGANTVRIVLSNGNQYTKDDINSVKNIISLVSNHKMIAVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLYQGLPLIIGEFGGYHQGADVDETEIMRYGQSKSVGWLAWSWYGNSSNLNYLDLVTGPNGNLTDWGRTVVEGANGIKETSKKAGIF

SEQ ID NO: 48 - protein, 296 aa, Artificial Sequence
  mature protein sequence, baed on the predicted cleavage of the naturally occurring sequence
ASGFYVSGTNLYDSTGKPFVMRGVNHAHTWYKNDLYTAIPAIAKTGANTVRIVLSNGNQYTKDDINSVKNIISLVSNHKMIAVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLYQGLPLIIGEFGGYHQGADVDETEIMRYGQSKSVGWLAWSWYGNSSNLNYLDLVTGPNGNLTDWGRTVVEGANGIKETSKKAGIF

SEQ ID NO: 49 - DNA, 987 bp, Artificial Sequence
  synthetic construct
gtgagaagcaaaaaattgtggatcagcttgttgtttgcgttaacgttaatctttacgatggcgttcagcaacatgagcgcgcaggctgctggaaaaatggcgacaggcttttatgtttcaggcaacaaactgtatgatagcacaggcaaaccgtttgttatgagaggcgttaatcatggccatagctggtttaaaaacgatctgaatacagcgattccggctattgcaaaaacaggcgcaaatacagttagaattgttctgtcaaatggcagcctgtatacgaaagatgatctgaatgcagtcaaaaacatcatcaatgtcgtcaaccagaacaaaatgattgcagttctggaagttcatgatgcaacgggcaaagatgattacaattcactggatgcagcagtcaactattggatctcaattaaagaagcgctgatcggcaaagaagatcgcgttattgttaatattgcgaacgaatggtatggcacatggaatggctcagcatgggcagatggctacaaaaaagcaattccgaaactgagaaatgcaggcatcaaaaacacactgattgttgatgcggcaggctggggacaatttccgcaatcaattgttgattatggccaaagcgtttttgcagcagatagccagaaaaatacagtctttagcatccatatgtacgaatacgctggaaaagatgcagcaacagttaaagcgaatatggaaaacgtcctgaataaaggcctggcactgattattggcgaatttggcggatatcatacaaatggcgacgttgatgaatatgcgattatgagatatggccaagaaaaaggcgttggctggcttgcatggtcatggtatggaaattcatcaggccttaactatctggatatggcaacaggaccgaatggatcactgacatcatttggcaatacagtcgtcaatgatacgtatggaatcaaaaatacgagccagaaagctggcatcttt

SEQ ID NO: 50 - protein, 329 aa, Artificial Sequence
  precursor protein expressed from synthetic construct
MRSKKLWISLLFALTLIFTMAFSNMSAQAAGKMATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 51 - protein, 300 aa, Artificial Sequence
  mature protein expressed from synthetic construct
AGKMATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 52 - protein, 297 aa, Artificial Sequence
  mature protein sequence, based on the predicted cleavage of the naturally occurring sequence
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 53 - DNA, 984 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaacaggcttttatgtttcaggcacaacactgtatgattcaacaggcaaaccgtttgttatgagaggcgttaatcatagccatacgtggtttaaaaacgatctgaatgcagcaattccggcaatcgcaaaaacaggcgcaaatacagttagaattgttctgtcaaatggcgtccagtatacaagagatgatgtcaatagcgtcaaaaacattatcagcctggtcaaccagaacaaaatgattgcagttctggaagttcatgatgcgacaggcaaagatgattatgcatcactggatgcagcagtcaattattggattagcattaaagatgcgctgatcggcaaagaagatcgcgttattgttaatattgcgaacgaatggtatggcacatggaatggctcagcatgggcagatggctataaacaagcgattccgaaactgagaaatgcaggcattaaaaacacactgattgttgatgcggcaggctggggacaatgtccgcaatcaattgttgattatggccaatcagtttttgcagcggatagcctgaaaaacacaatctttagcatccatatgtatgaatatgcaggcggaacggatgcaattgtcaaaagcaatatggaaaacgtcctgaataaaggcctgccgctgattattggcgaatttggcggacaacatacaaatggcgacgttgatgaacatgcaattatgagatatggccaacaaaaaggcgttggctggcttgcatggtcatggtatggaaataattcagaactgagctatctggatctggcaacaggaccggcaggctcactgacatcaattggaaatacaattgtgaacgatccgtatggcattaaagcgacatcaaaaaaagcaggcattttt

SEQ ID NO: 54 - protein, 328 aa, Artificial Sequence
  precursor protein expressed from synthetic construct
MRSKKLWISLLFALTLIFTMAFSNMSAQAAGKATGFYVSGTTLYDSTGKPFVMRGVNHSHTWFKNDLNAAIPAIAKTGANTVRIVLSNGVQYTRDDVNSVKNIISLVNQNKMIAVLEVHDATGKDDYASLDAAVNYWISIKDALIGKEDRVIVNIANEWYGTWNGSAWADGYKQAIPKLRNAGIKNTLIVDAAGWGQCPQSIVDYGQSVFAADSLKNTIFSIHMYEYAGGTDAIVKSNMENVLNKGLPLIIGEFGGQHTNGDVDEHAIMRYGQQKGVGWLAWSWYGNNSELSYLDLATGPAGSLTSIGNTIVNDPYGIKATSKKAGIF

SEQ ID NO: 55 - protein, 299 aa, Artificial Sequence
  mature protein expressed from synthetic construct
AGKATGFYVSGTTLYDSTGKPFVMRGVNHSHTWFKNDLNAAIPAIAKTGANTVRIVLSNGVQYTRDDVNSVKNIISLVNQNKMIAVLEVHDATGKDDYASLDAAVNYWISIKDALIGKEDRVIVNIANEWYGTWNGSAWADGYKQAIPKLRNAGIKNTLIVDAAGWGQCPQSIVDYGQSVFAADSLKNTIFSIHMYEYAGGTDAIVKSNMENVLNKGLPLIIGEFGGQHTNGDVDEHAIMRYGQQKGVGWLAWSWYGNNSELSYLDLATGPAGSLTSIGNTIVNDPYGIKATSKKAGIF

SEQ ID NO: 56 - protein, 296 aa, Artificial Sequence
  mature protein sequence, based on the predicted cleavage of the naturally occurring sequence
ATGFYVSGTTLYDSTGKPFVMRGVNHSHTWFKNDLNAAIPAIAKTGANTVRIVLSNGVQYTRDDVNSVKNIISLVNQNKMIAVLEVHDATGKDDYASLDAAVNYWISIKDALIGKEDRVIVNIANEWYGTWNGSAWADGYKQAIPKLRNAGIKNTLIVDAAGWGQCPQSIVDYGQSVFAADSLKNTIFSIHMYEYAGGTDAIVKSNMENVLNKGLPLIIGEFGGQHTNGDVDEHAIMRYGQQKGVGWLAWSWYGNNSELSYLDLATGPAGSLTSIGNTIVNDPYGIKATSKKAGIF

SEQ ID NO: 57 - DNA, 987 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcaacaggcttttatgtttcaggaacaaaactttatgatagcacgggaaaaccgtttgtgatgagaggcgttaatcactcacatacatggtttaagaatgatctgaatgcagctatccctgcgattgcgaagacaggcgcaaacacggttagaattgttctgtcaaacggcgttcaatatacgagagatgatgttaattcagtcaagaatatcatttcactggtgaatcaaaataagatgattgcagttctggaagttcatgatgctacaggaaaagacgattatgcatcactggatgcagcaattaactattggatttcaattaaagatgcactgattggcaaagaagatagagttattgtgaacattgcaaatgaatggtatggcacatggaatggctcagcatgggcagatggatataaacaagctattcctaaactgagaaatgcgggcatcaaaaatacgctgatcgtggatgcggctggctggggccaatatccgcaatcaattgttgattacggccagtcagtttttgcagcagattcactgaagaacacagtgtttagcatccatatgtatgaatatgcaggcggcacagatgcaatggttaaagctaatatggaaggagttctgaataaaggcctgccgctgattattggagaatttggcggacaacatacaaatggcgatgttgacgaactggcaattatgagatatggccaacaaaaaggcgtgggatggctggcatggtcatggtacggcaacaacagcgatctgtcatatcttgatctggcaacgggaccgaatggatcactgacaacgtttggaaatacagtggtgaacgatacgaacggaattaaggcaacgagcaagaaggcgggaatttttcaa

SEQ ID NO: 58 - protein, 329 aa, Artificial Sequence
  precursor protein expressed from synthetic construct
MRSKKLWISLLFALTLIFTMAFSNMSAQAAGKATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 59 - protein, 300 aa, Artificial Sequence
  mature protein expressed from synthetic construct
AGKATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 60 - protein, 297 aa, Artificial Sequence
  mature protein sequence, based on the predicted cleavage of the naturally occurring sequence
ATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 61 - DNA, 984 bp, Paenibacillus sp. N021
atggtcaatctgaagaaatgtacgatctttacgttgattgctgcgctcatgttcatggctctggggagtgttacgcccaaggcagctgctgcatccggtttttatgtaagcgggaataagttatatgactcgactggcaagccttttgtcatgagaggaatcaatcacggccattcctggttcaaaaatgatctgaatacagccatacctgctattgcgaaaacaggcgccaacacggtacgaattgttctctcgaatggaacactgtacaccaaagatgatctgaattcagttaaaaacataatcaatctggtcaatcagaataagatgatcgccgtgcttgaagtgcatgatgcaacaggcaaagacgattataactcgctggatgcagccgtgaattactggatcagcatcaaagaagcgttgattggcaaggaagatcgagtgatcgttaatatcgccaacgaatggtatggaacctggaacggcagcgcttgggcagacggttacaaaaaggctattccgaagctcagaaacgcaggcatcaaaaatacgttgattgttgatgctgcaggctggggtcaatatccacaatcgattgtcgattatggtcaaagcgtattcgcaacagatacgctcaaaaatacggtgttttccattcatatgtatgaatatgcgggtaaggatgcggcaacggtgaaagctaatatggagaatgtgctgaacaaaggacttgcagtaatcattggtgagttcggtggatatcacacaaatggtgatgtggatgaatatgccattatgagatatggacaagagaagggtgtaggctggcttgcatggtcatggtacggcaacagttccggtctgggttatctggatctggctaccggtccgaacggaagtctcacaagttatggcaatacggtagttaatgacacatacggaatcaaaaatacgtcccaaaaagcagggatatttcaatag

SEQ ID NO: 62 - protein, 327 aa, Paenibacillus sp. N021
MVNLKKCTIFTLIAALMFMALGSVTPKAAAASGFYVSGNKLYDSTGKPFVMRGINHGHSWFKNDLNTAIPAIAKTGANTVRIVLSNGTLYTKDDLNSVKNIINLVNQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFATDTLKNTVFSIHMYEYAGKDAATVKANMENVLNKGLAVIIGEFGGYHTNGDVDEYAIMRYGQEKGVGWLAWSWYGNSSGLGYLDLATGPNGSLTSYGNTVVNDTYGIKNTSQKAGIFQ

SEQ ID NO: 63 - protein, 297 aa, Paenibacillus sp. N021
ASGFYVSGNKLYDSTGKPFVMRGINHGHSWFKNDLNTAIPAIAKTGANTVRIVLSNGTLYTKDDLNSVKNIINLVNQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFATDTLKNTVFSIHMYEYAGKDAATVKANMENVLNKGLAVIIGEFGGYHTNGDVDEYAIMRYGQEKGVGWLAWSWYGNSSGLGYLDLATGPNGSLTSYGNTVVNDTYGIKNTSQKAGIFQ

SEQ ID NO: 64 - DNA, 987 bp, Artificial Sequence
  synthetic construct
gtgagaagcaaaaaattgtggatcagcttgttgtttgcgttaacgttaatctttacgatggcgttcagcaacatgagcgcgcaggctgctggaaaagcatcaggcttttatgtttcaggcaataaactttatgattcaacaggaaaaccgtttgttatgagaggaattaatcacggacattcatggttcaaaaatgatcttaacacagctattccggcgattgcgaagacaggcgcaaatacagttagaattgttctgtcaaatggcacgctgtacacaaaggacgatctgaacagcgttaaaaacatcattaatctggttaatcaaaataagatgattgcagttctggaagtccatgatgctacaggcaaagacgattacaattcactggatgctgcagtcaattactggatttcaattaaagaagcactgattggaaaagaggacagagttattgttaatatcgcaaatgaatggtatggaacatggaatggcagcgcatgggcagatggctataagaaagcaattccgaaactgagaaacgcaggcatcaagaacacgcttatcgttgatgcagcaggctggggacaatatccgcaatcaattgttgattatggccaaagcgtttttgcaacagacacactgaaaaacacagttttctcaattcatatgtacgaatatgccggaaaggatgcggcaacggttaaagcaaatatggaaaatgttctgaataaaggcctggcagttattatcggcgaatttggcggctatcatacgaatggcgatgttgacgaatacgcgatcatgagatatggacaggagaaaggcgttggctggcttgcgtggtcatggtacggaaatagctcaggactgggctatctggatcttgcaacgggaccgaacggctcacttacatcatatggcaacacggtcgtgaatgatacatacggcattaagaatacatcacaaaaagccggcatttttcaa

SEQ ID NO: 65 - protein, 329 aa, Artificial Sequence
  precursor protein expressed from synthetic construct
MRSKKLWISLLFALTLIFTMAFSNMSAQAAGKASGFYVSGNKLYDSTGKPFVMRGINHGHSWFKNDLNTAIPAIAKTGANTVRIVLSNGTLYTKDDLNSVKNIINLVNQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFATDTLKNTVFSIHMYEYAGKDAATVKANMENVLNKGLAVIIGEFGGYHTNGDVDEYAIMRYGQEKGVGWLAWSWYGNSSGLGYLDLATGPNGSLTSYGNTVVNDTYGIKNTSQKAGIFQ

SEQ ID NO: 66 - protein, 300 aa, Artificial Sequence
  mature protein expressed from synthetic construct
AGKASGFYVSGNKLYDSTGKPFVMRGINHGHSWFKNDLNTAIPAIAKTGANTVRIVLSNGTLYTKDDLNSVKNIINLVNQNKMIAVLEVHDATGKDDYNSLDAAVNYWISIKEALIGKEDRVIVNIANEWYGTWNGSAWADGYKKAIPKLRNAGIKNTLIVDAAGWGQYPQSIVDYGQSVFATDTLKNTVFSIHMYEYAGKDAATVKANMENVLNKGLAVIIGEFGGYHTNGDVDEYAIMRYGQEKGVGWLAWSWYGNSSGLGYLDLATGPNGSLTSYGNTVVNDTYGIKNTSQKAGIFQ

SEQ ID NO: 67 - protein, 297 aa, Artificial Sequence; identical to SEQ ID NO: 63
  mature protein sequence, based on the predicted cleavage of the naturally occurring sequence.

SEQ ID NO: 68 - protein, 296 aa, Paenibacillus sp. FSL R5-192
ATGFYVSGNKLYDSTGKAFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 69 - protein, 296 aa, Paenibacillus sp. PAMC 26794
ATGFYVSGNKLYDSTGKAFVMRGVNHGHSWFKNDLNTAIPAIAKTGANTVRIVLSNGSLYTKDDLNAVKNIINVVNQNKMIAVLEVHDATGKEDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 70 - protein, 296 aa, unknown
  Paenibacillus sp.
ATGFYVSGGKLYDSTGKAFVMRGVNHGHSWFKNDLNTAIPAIAKTGANTVRIVLSNGVQYTKDDLNAVKNIINVISANKMIAVLEVHDATGKDDYNSLDAAVNYWISIKEALIGKEDRVIVNIANEWYGTWNGSAWADGYKKAIPKLRNAGINNTLIVDAAGWGQYPQSIVDYGQSVFAADSQKNTVFSIHMYEYAGKDAATVKANMESVLNKGLALIIGEFGGYHTNGDVDEYAIMKYGQEKGVGWLAWSWYGNNSDLSYLDLAMGPNGSLTSFGNTVVNDTYGIKNTSQKAGIY

SEQ ID NO: 71 - protein, 296 aa, Paenibacillus sp. A9
ATGFYVSGTKLYDSTGKPFAMRGINHAHTWYKNDLNTAIPAIARTGANTVRIVLSNGMQYTKDDVNSVKNIISLVNQNKMVAVLEVHDATGKDDYNSLDAAVNYWISIKDALIGKEDRVIVNIANEWYGTWNGSAWADGYKQAIPKLRNAGIKNTLIVDAAGWGQYPQSIVDYGQSVFAADSQRNTVFSIHMYEYAGKDAATVKANIDGVLNKGLPVIIGEFGGYHTNGDVDEYAIMRYGQEKGIGWLAWSWYGNSTNLNYLDLATGPNGSLTSFGNTVVNDPSGIKATSQKAGIF

SEQ ID NO: 72 - protein, 296 aa, unknown
  Paenibacillus sp.
ASGFYVSGTKLYDSTGNPFVMRGVNHAHTWYKNDLYTAIPAIAKTGANTVRIVLSNGTQYTKDDINSVKNIISLVTSYKMIPVLEVHDATGKDDYASLDAAVNYWISIKDALIGKEDRVIVNIANEWYGSWNGGGWADGYKQAIPKLRNAGIKNTLIVDCAGWGQYPQSINDFGKSVFAADSLKNTVFSIHMYEFAGKDVQTVRTNIDNVLNQGLPLIIGEFGGYHQGADVDETEIMRYGQSKGIGWLAWSWYGNSSNLSYLDLVTGPNGNLTDWGRTVVEGTNGIKETSKKAGIY

SEQ ID NO: 73 - protein, 296 aa, Paenibacillus sp._HGF5
ATGFYVNGTKLYDSTGKAFVMRGVNHPHTWYKNDLNAAIPAIAQTGANTVRVVLSNGSQWIKDDLNAVNSIISLVSQHQMIAVLEVHDATGKDDDASLEAAVDYWIGIKEALIGKEDRVIVNIANEWYGNWNSSGWAEGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY

SEQ ID NO: 74 - protein, 298 aa, Paenibacillus sp. HW567
VKGFYVSGTKLYDATGSPFVMRGVNHAHTWYKNDLATAIPAIAATGSNTIRIVLSNGSKWSLDSLSDVKNILALCDQYKLTAMLEVHDATGSDNASDLNAAVNYWISIKDALIGKEDRVIVNIANEWFGSWGTASWASAYQSAIPALRAAGIKNTLVVDAAGWGQYPTSIFTSGNAVFNSDPLRNTIFSIHMYEYAGGTAATVKSNIDNALAIGVPVIVGEFGFKHTGGDVDEATIMSYSQEKGVGWLAWSWYGNGGGVEYLDLSNGPSGNLTDWGKTVVNGSYGTLATSVLGKIYTT

SEQ ID NO: 75 - protein, 299 aa, Bacillus Lentus
ASGFYVSGTILCDSTGNPFKIRGINHAHSWFKNDSATAMEAIAATGANTVRIVLSNGQQYAKDDANTVSNLLSLANQHKLIAILEVHDATGSDSVSALDHAVDYWIEMKNVLVGKEDRVLINIANEWYGTWDSNGWADGYKSAIPKLRNAGINHTLIVDAAGWGQYPQSIVDKGNEVFNSDPLRNTIFSIHMYEYAGGNADMVRANIDQVLNKGLAVIIGEFGHYHTGGDVDETAIMSYTQQKGVGWLAWSWKGNGAEWLYLDLSYDWAGNHLTEWGETIVNGANGLKATSTRAPIFGN

SEQ ID NO: 76 - protein, 324 aa, Bacillus nealsonii
ASGFYVSGTTLYDATGKPFTMRGVNHAHSWFKEDSAAAIPAIAATGANTVRIVLSDGGQYTKDDINTVKSLLSLAEKINLHSGVMTHRKDDVESLNRAVDYWISLKDTLIGKEDKVIINIANEWYGTWDGAAWAAGYKQAIPKLRNAGLNHTLIIDSAGWGQYPASIHNYGKEVFNADPLKNTMFSIHMYEYAGGDAATVKSNIDGVLNQGLALIIGEFGQKHTNGDVDEATIMSYSQQKNIGWLAWSWKGNSTDWSYLDLSNDWSGNSLTDWGNTVVNGANGLKATSKLSGVFGSSAGTNNILYDFESGNQNWTGSNIAGGPW

SEQ ID NO: 77 - protein, 299 aa, Bacillus sp. JAMB-602
NSGFYVSGTTLYDANGNPFVMRGINHGHAWYKDQATTAIEGIANTGANTVRIVLSDGGQWTKDDIQTVRNLISLAEDNNLVAVLEVHDATGYDSIASLNRAVDYWIEMRSALIGKEDTVIINIANEWFGSWDGAAWADGYKQAIPRLRNAGLNNTLMIDAAGWGQFPQSIHDYGREVFNADPQRNTMFSIHMYEYAGGNASQVRTNIDRVLNQDLALVIGEFGHRHTNGDVDESTIMSYSEQRGVGWLAWSWKGNGPEWEYLDLSNDWAGNNLTAWGNTIVNGPYGLRETSKLSTVFTG

SEQ ID NO: 78 - protein, 300 aa, Unknown
  Bacillus sp.
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTG

SEQ ID NO: 79 - protein, 294 aa, Bacillus agaradhaerens
  misc_feature (294)..(294): Xaa can be any naturally occurring amino acid
GFSVDGNTLYDANGQPFVMRGINHGHAWYKDTASTAIPAIAEQGANTIRIVLSDGGQWEKDDIDTIREVIELAEQNKMVAVVEVHDATGRDSRSDLNRAVDYWIEMKDALIGKEDTVIINIANEWYGSWDGSAWADGYIDVIPKLRDAGLTHTLMVDAAGWGQYPQSIHDYGQDVFNADPLKNTMFSIHMYEYAGGDANTVRSNIDRVIDQDLALVIGEFGHRHTDVDEDTILSYSEETGTGWLAWSWKGNSTSWDYLDLSEDWAGQHLTDWGNRIVHGADGLQETSKPSTVFX

SEQ ID NO: 80 - protein, 301 aa, Artificial sequence
  Consensus sequence
  misc_feature (1)..(1): Xaa can be any naturally occurring amino acid
  misc_feature (59)..(59): Xaa can be any naturally occurring amino acid
  misc_feature (77)..(77): Xaa can be any naturally occurring amino acid
  misc_feature (235)..(235): Xaa can be any naturally occurring amino acid
  misc_feature (261)..(261): Xaa can be any naturally occurring amino acid
  misc_feature (272)..(272): Xaa can be any naturally occurring amino acid
  misc_feature (290)..(290): Xaa can be any naturally occurring amino acid
  misc_feature (293)..(293): Xaa can be any naturally occurring amino acid
  misc_feature (299)..(301): Xaa can be any naturally occurring amino acid
XATGFYVSGTKLYDSTGKPFVMRGVNHGHTWFKNDLNTAIPAIAKTGANTVRIVLSNGXQYTKDDLNSVKNIISLVXQNKMIAVLEVHDATGKDDYASLDAAVNYWISIKEALIGKEDRVIVNIANEWYGTWNGSAWADGYKQAIPKLRNAGIKNTLIVDAAGWGQYPQSIVDYGQSVFAADSLKNTVFSIHMYEYAGKDAATVKANMDNVLNKGLALIIGEFGGYHTNGDVDEXAIMRYGQEKGVGWLAWSWYGNSSDLXYLDLATGPNGXSLTSWGNTVVNGTYGIKXTSXKAGIFXXX

SEQ ID NO: 81 - protein, 440 aa, Paenibacillus mucilaginosus
ATGMYVSGTTVYDANGKPFVMRGINHPHAWYKNDLATAIPAIAATGANSVRIVLSNGSQWSKDSLASIQNIIALCEQYRMIAILEVHDATGSDSYTALDNAVNYWIEMKSALIGKERTVIINIANEWYGTWDASGWANGYKQAIPKLRSAGLDHLLMVDAAGWGQYPASIHTMGKEVLAADPRKNTMFSIHMYEYAGGTADQVRSNIDGVLNQGLAVVVGEFGPKHSNGEVDEATIMSYSQQKGVGWLVWSWYGNSSDLNYLDVATGPSGSLTSWGNTVVNGTNGIKATSALASVFGTGTGGGTTTYVKLQNRASGLYADSWGRTANGNNVALSGSGTSNNQQWVVEAAGTYVKIKNRANGLYLDGMGRTANGSAASFWSGSSSYNQQWTKEDAGSGYVRFKNRATGLYLDTVGRTTAGSDLGQWAYSTSYNQQWKLVNP

SEQ ID NO: 82 - protein, 301 aa, Artificial sequence
  Consensus sequence
  misc_feature (1)..(1): Xaa can be any naturally occurring amino acid
  misc_feature (77)..(77): Xaa can be any naturally occurring amino acid
  misc_feature (235)..(235): Xaa can be any naturally occurring amino acid
  misc_feature (243)..(243): Xaa can be any naturally occurring amino acid
  misc_feature (273)..(273): Xaa can be any naturally occurring amino acid
  misc_feature (286)..(286): Xaa can be any naturally occurring amino acid
  misc_feature (290)..(290): Xaa can be any naturally occurring amino acid
  misc_feature (299)..(301): Xaa can be any naturally occurring amino acid
XATGFYVSGTKLYDSTGKPFVMRGVNHAHTWYKNDLNTAIPAIAKTGANTVRIVLSNGSQYTKDDLNSVKNIISLVXQNKMIAVLEVHDATGKDDYASLDAAVNYWISIKDALIGKEDRVIVNIANEWYGTWNGSAWADGYKQAIPKLRNAGIKNTLIVDAAGWGQYPQSIVDYGQSVFAADSLKNTVFSIHMYEYAGKDAATVKANMDNVLNKGLALIIGEFGGYHTNGDVDEXAIMRYGQXKGVGWLAWSWYGNSSDLNYLDLATGPNGSXLTSWGNTVVNGTXGIKXTSKKAGIFXXX

SEQ ID NO: 83 - protein, 50 aa, Paenibacillus amylolyticus
ATGFYVSGNKLYDSTGKAFVMRGVNHGHSWFKNDLNTAIPAIAKTGANTV

SEQ ID NO: 84 - protein, 50 aa, Paenibacillus tundrae
ATGFYVSGGKLYDSTGKAFVMRGVNHGHSWFKNDLNTAIPAIAKTGANTV

SEQ ID NO: 85 - protein, 50 aa, Paenibacillus pabuli
AAGFYVSGNKLYDSTGKAFVMRGVNHSHTWFKNDLNTAIPAIAKTGANTV

SEQ ID NO: 86 - protein, 50 aa, Paenibacillus hunanensis
ATGFYVSGTKLYDSTGKPFVMRGVNHSHTWFKNDLNAAIPAIAKTGANTV

SEQ ID NO: 87 - protein, 50 aa, Paenibacillus sp. A1
MATGFYVSGNKLYDSTGKPFVMRGVNHGHSWFKNDLNTAIPAIAKTGANT

SEQ ID NO: 88 - protein, 50 aa, Paenibacillus sp_CH-3
ATGFYVSGTTLYDSTGKPFVMRGVNHSHTWFKNDLNAAIPAIAKTGANTV

SEQ ID NO: 89 - protein, 50 aa, Paenibacillus sp_PAMC26794; identical to SEQ ID NO: 83

SEQ ID NO: 90 - protein, 50 aa, Bacillus circulans
ATGFYVNGGKLYDSTGKPFYMRGINHGHSWFKNDLNTAIPAIAKTGANTV

SEQ ID NO: 91 - protein, 50 aa, Paenibacillus sp.A9
ATGFYVSGTKLYDSTGKPFAMRGINHAHTWYKNDLNTAIPAIARTGANTV

SEQ ID NO: 92 - protein, 50 aa, Bacillus circulans
ATGFYVNGTKLYDSTGKAFVMRGVNHPHTWYKNDLNAAIPAIAQTGANTV

SEQ ID NO: 93 - protein, 50 aa, Paenibacillus polymyxa
ASGFYVSGTKLYDSTGKPFVMRGVNHAHTWYKNDLYTAIPAIAQTGANTV

SEQ ID NO: 94 - protein, 50 aa, Paenibacillus sp. HGF5; identical to SEQ ID NO: 92

SEQ ID NO: 95 - protein, 50 aa, unknown
  Paenibacillus sp.
ASGFYVSGTKLYDSTGNPFVMRGVNHAHTWYKNDLYTAIPAIAKTGANTV

SEQ ID NO: 96 - protein, 50 aa, Paenibacillus polymyxa
ASGFYVSGTNLYDSTGKPFVMRGVNHAHTWYKNDLYTAIPAIAKTGANTV

SEQ ID NO: 97 - protein, 50 aa, Paenibacillus sp. HW567
VKGFYVSGTKLYDATGSPFVMRGVNHAHTWYKNDLATAIPAIAATGSNTI

SEQ ID NO: 98 - protein, 50 aa, Paenibacillus mucilaginosus
ATGMYVSGTTVYDANGKPFVMRGINHPHAWYKNDLATAIPAIAATGANSV

SEQ ID NO: 99 - protein, 50 aa, Bacillus circulans
ASGFYVSGTKLLDATGQPFVMRGVNHAHTWYKDQLSTAIPAIAKTGANTI

SEQ ID NO: 100 - protein, 50 aa, Bacillus nealsonii
ASGFYVSGTTLYDATGKPFTMRGVNHAHSWFKEDSAAAIPAIAATGANTV

SEQ ID NO: 101 - protein, 50 aa, Bacillus sp. JAMB-602
NSGFYVSGTTLYDANGNPFVMRGINHGHAWYKDQATTAIEGIANTGANTV

SEQ ID NO: 102 - protein, 50 aa, Artificial sequence
  Consensus sequence
  misc_feature (1)..(1): Xaa can be any naturally occurring amino acid
XATGFYVSGTKLYDSTGKPFVMRGVNHAHTWYKNDLNTAIPAIAKTGANT

SEQ ID NO: 103 - protein, 46 aa, Paenibacillus amylolyticus
SWYGNSSGLNYLDMATGPNGSLTSFGNTVVNDTYGIKKTSQKAGIF

SEQ ID NO: 104 - protein, 46 aa, Paenibacillus tundrae
SWYGNSSDLNYLDLATGPNGSLTSFGNTVVNDTYGIKNTSKKAGIY

SEQ ID NO: 105 - protein, 46 aa, Paenibacillus pabuli
SWYGNNSDLNYLDLATGPNGTLTSFGNTVVYDTYGIKNTSVKAGIY

SEQ ID NO: 106 - protein, 47 aa, Paenibacillus hunanensis
SWYGNNSDLSYLDLATGPNGSLTTFGNTVVNDTNGIKATSKKAGIFQ

SEQ ID NO: 107 - protein, 46 aa, Paenibacillus sp. A1
SWYGNSSGLNYLDMATGPNGSLTSFGNTVVNDTYGIKNTSQKAGIF

SEQ ID NO: 108 - protein, 46 aa, Paenibacillus sp. CH-3
SWYGNNSELSYLDLATGPAGSLTSIGNTIVNDPYGIKATSKKAGIF

SEQ ID NO: 109 - protein, 46 aa, Paenibacillus sp. PAMC 26794; identical to SEQ ID NO: 107

SEQ ID NO: 110 - protein, 49 aa, Bacillus nealsonii
SWKGNSTDWSYLDLSNDWSGNSLTDWGNTVVNGANGLKATSKLSGVFGS

SEQ ID NO: 111 - protein, 49 aa, Bacillus sp. JAMB-602
SWKGNGPEWEYLDLSNDWAGNNLTAWGNTIVNGPYGLRETSKLSTVFTG

SEQ ID NO: 112 - protein, 50 aa, Artificial sequence
  Consensus sequence
  misc_feature (22)..(22): Xaa can be any naturally occurring amino acid
  misc_feature (35)..(35): Xaa can be any naturally occurring amino acid
  misc_feature (39)..(39): Xaa can be any naturally occurring amino acid
  misc_feature (48)..(50): Xaa can be any naturally occurring amino acid
SWYGNSSDLNYLDLATGPNGSXLTSWGNTVVNGTXGIKXTSKKAGIFXXX

SEQ ID NO: 113 - protein, 300 aa, Artificial sequence
  Consensus sequence
  misc_feature (1)..(1): Xaa can be any naturally occurring amino acid
  misc_feature (28)..(28): Xaa can be any naturally occurring amino acid
  misc_feature (59)..(59): Xaa can be any naturally occurring amino acid
  misc_feature (74)..(74): Xaa can be any naturally occurring amino acid
  misc_feature (77)..(77): Xaa can be any naturally occurring amino acid
  misc_feature (143)..(143): Xaa can be any naturally occurring amino acid
  misc_feature (184)..(184): Xaa can be any naturally occurring amino acid
  misc_feature (259)..(259): Xaa can be any naturally occurring amino acid
  misc_feature (276)..(276): Xaa can be any naturally occurring amino acid
  misc_feature (289)..(289): Xaa can be any naturally occurring amino acid
  misc_feature (292)..(292): Xaa can be any naturally occurring amino acid
  misc_feature (298)..(300): Xaa can be any naturally occurring amino acid
XATGFYVSGTKLYDSTGKPFVMRGVNHXHTWFKNDLNTAIPAIAKTGANTVRIVLSNGXQYTKDDLNSVKNIIXLVXQNKMIAVLEVHDATGKDDYNSLDAAVNYWISIKEALIGKEDRVIVNIANEWYGTWNGSAWADGYKXAIPKLRNAGIKNTLIVDAAGWGQYPQSIVDYGQSVFAADSXKNTVFSIHMYEYAGKDAATVKANMENVLNKGLALIIGEFGGYHTNGDVDEYAIMRYGQEKGVGWLAWSWYGNSSXLNYLDLATGPNGSLTSXGNTVVNDTYGIKXTSXKAGIFXXX

SEQ ID NO: 114 - protein, 46 aa, Bacillus circulans
SWYGNSSGLNYLDLATGPNGSLTSYGNTVVNDTYGIKNTSQKAGIF

SEQ ID NO: 115 - protein, 46 aa, Paenibacillus sp. A9
SWYGNSTNLNYLDLATGPNGSLTSFGNTVVNDPSGIKATSQKAGIF

SEQ ID NO: 116 - protein, 46 aa, Bacillus circulans
SWYGNSPELNDLDLAAGPSGNLTGWGNTVVHGTDGIQQTSKKAGIY

SEQ ID NO: 117 - protein, 46 aa, Paenibacillus polymyxa
SWYGNSSNLSYLDLVTGPNGNLTDWGKTVVNGSNGIKETSKKAGIY

SEQ ID NO: 118 - protein, 46 aa, Paenibacillus sp. HGF5; identical to SEQ ID NO: 116

SEQ ID NO: 119 - protein, 46 aa, unknown
  Paenibacillus sp.
SWYGNSSNLSYLDLVTGPNGNLTDWGRTVVEGTNGIKETSKKAGIY

SEQ ID NO: 120 - protein, 46 aa, Paenibacillus polymyxa
SWYGNSSNLNYLDLVTGPNGNLTDWGRTVVEGANGIKETSKKAGIF

SEQ ID NO: 121 - protein, 49 aa, Paenibacillus sp. HW567
SWYGNGGGVEYLDLSNGPSGNLTDWGKTVVNGSYGTLATSVLGKIYTTP

SEQ ID NO: 122 - protein, 49 aa, Paenibacillus mucilaginosus
SWYGNSSDLNYLDVATGPSGSLTSWGNTVVNGTNGIKATSALASVFGTG

SEQ ID NO: 123 - protein, 50 aa, Bacillus circulans
SWKGNSSDLAYLDMTNDWAGNSLTSFGNTVVNGSNGIKATSVLSGIFGGV

SEQ ID NO: 124 - protein, 299 aa, Bacillus circulans
ASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGG

SEQ ID NO: 125 - protein, 299 aa, Paenibacillus sp. HW567
VKGFYVSGTKLYDATGSPFVMRGVNHAHTWYKNDLATAIPAIAATGSNTIRIVLSNGSKWSLDSLSDVKNILALCDQYKLTAMLEVHDATGSDNASDLNAAVNYWISIKDALIGKEDRVIVNIANEWFGSWGTASWASAYQSAIPALRAAGIKNTLVVDAAGWGQYPTSIFTSGNAVFNSDPLRNTIFSIHMYEYAGGTAATVKSNIDNALAIGVPVIVGEFGFKHTGGDVDEATIMSYSQEKGVGWLAWSWYGNGGGVEYLDLSNGPSGNLTDWGKTVVNGSYGTLATSVLGKIYTTP

SEQ ID NO: 126 - protein, 296 aa, Bacillus nealsonii
ASGFYVSGTTLYDATGKPFTMRGVNHAHSWFKEDSAAAIPAIAATGANTVRIVLSDGGQYTKDDINTVKSLLSLAEKINLHSGVMTHRKDDVESLNRAVDYWISLKDTLIGKEDKVIINIANEWYGTWDGAAWAAGYKQAIPKLRNAGLNHTLIIDSAGWGQYPASIHNYGKEVFNADPLKNTMFSIHMYEYAGGDAATVKSNIDGVLNQGLALIIGEFGQKHTNGDVDEATIMSYSQQKNIGWLAWSWKGNSTDWSYLDLSNDWSGNSLTDWGNTVVNGANGLKATSKLSGVFGS

SEQ ID NO: 127 - protein, 298 aa, Paenibacillus mucilaginosus
ATGMYVSGTTVYDANGKPFVMRGINHPHAWYKNDLATAIPAIAATGANSVRIVLSNGSQWSKDSLASIQNIIALCEQYRMIAILEVHDATGSDSYTALDNAVNYWIEMKSALIGKERTVIINIANEWYGTWDASGWANGYKQAIPKLRSAGLDHLLMVDAAGWGQYPASIHTMGKEVLAADPRKNTMFSIHMYEYAGGTADQVRSNIDGVLNQGLAVVVGEFGPKHSNGEVDEATIMSYSQQKGVGWLVWSWYGNSSDLNYLDVATGPSGSLTSWGNTVVNGTNGIKATSALASVFGT

SEQ ID NO: 128 - protein, 299 aa, Paenibacillus mucilaginosus
ATGMYVSGTTVYDANGKPFVMRGINHPHAWYKNDLATAIPAIAATGANSVRIVLSNGSQWSKDSLASIQNIIALCEQYRMIAILEVHDATGSDSYTALDNAVNYWIEMKSALIGKERTVIINIANEWYGTWDASGWANGYKQAIPKLRSAGLDHLLMVDAAGWGQYPASIHTMGKEVLAADPRKNTMFSIHMYEYAGGTADQVRSNIDGVLNQGLAVVVGEFGPKHSNGEVDEATIMSYSQQKGVGWLVWSWYGNSSDLNYLDVATGPSGSLTSWGNTVVNGTNGIKATSALASVFGTG

(19) United States
(12) Patent Application Publication
(10) Pub. No.: US 2020/0277590 A1
(43) Pub. Date: Sep. 3, 2020

(54) PAENIBACILLUS AND BACILLUS SPP. MANNANASES

(71) Applicant: DANISCO US INC, Palo Alto, CA (US)

(72) Inventors: Christian D. Adams, San Francisco, CA (US); Roopa Santosh Ghirnikar, Sunnyvale, CA (US); Victoria Huang, Sunnyvale, CA (US); Marc Kolkman, Oegstgeest (NL); Zhen Qian, Shanghai (CN); Liling Jin, Shanghai (CN)

(21) Appl. No.: 16/577,096

(22) Filed: Sep. 20, 2019

Related U.S. Application Data
(63) Continuation of application No. 15/325,305, filed on Jan. 10, 2017, now abandoned.

(30) Foreign Application Priority Data
Jul. 11, 2014 (CN) PCT/CN2014/082034

(51) Int. Cl. C12N 9/24; A23L 29/00; A23K 20/189; C11D 3/386; C12C 5/00

(52) U.S. Cl. CPC C12N 9/2494; A23L 29/06; A23K 20/189; C11D 3/38681; A23V 2002/00; C12N 9/2488; C11D 3/38636; C12Y 302/01078; C12C 5/004

(57) ABSTRACT

The present disclosure relates to endo-beta-mannanases from Paenibacillus and Bacillus spp., polynucleotides encoding such endo-beta-mannanases, compositions containing such mannanases, and methods of use thereof. Compositions containing such endo-beta-mannanases are suitable for use as detergents and cleaning fabrics and hard surfaces, as well as a variety of other industrial applications.

### CROSS REFERENCE TO RELATED APPLICATION

This application is a Continuation of U.S. application Ser. No. 15/325,305, filed on Jan. 10, 2017, which is a 371 of International Application No. PCT/US15/40057, filed Jul. 10, 2015 and claims priority to International Application No. PCT/CN2014/082034, filed on Jul. 11, 2014, the contents of which are hereby incorporated herein by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named “40597-WO-PCT_Amended_Sequence_Listing_ST25.txt” created on Feb. 24, 2020 and having a size of 239 kilobytes and is filed concurrently with the specification. The sequence listing contained in this ASCII formatted document is part of the specification and is herein incorporated by reference in its entirety.

The present disclosure relates to endo-β-mannanases from Paenibacillus or Bacillus spp, polynucleotides encoding such endo-β-mannanases, compositions containing such mannanases, and methods of use thereof. Compositions containing such endo-β-mannanases are suitable for use as detergents and cleaning fabrics and hard surfaces, as well as a variety of other industrial applications.

Mannanase enzymes, including endo-β-mannanases, have been employed in detergent cleaning compositions for the removal of gum stains by hydrolyzing mannans. A variety of mannans are found in nature, such as, for example, linear mannan, glucomannan, galactomannan, and glucogalactomannan. Each such mannan is comprised of polysaccharides that containa β-1,4-linked backbone of mannose residues that may be substituted up to 33% with glucose residues (Yeoman et al., Adv Appl Microbiol, Elsivier). In galactomannans or glucogalactomannnans, galactose residues are linked in alpha-1,6-linkages to the mannan backbone (Moreira and Filho, Appl Microbiol Biotechnol, 79:165, 2008). Therefore, hydrolysis of mannan to its component sugars requires endo-1,4β-mannanases that hydrolyze the backbone linkages to generate short chain manno-oligosaccharides that are further degraded to monosaccharides by 1,4β-mannosidases.

Although endo-β-mannanases have been known in the art of industrial enzymes, there remains a need for further endo-β-mannanases that are suitable for particular conditions and uses.

In particular, the present disclosure provides a recombinant polypeptide or active fragment thereof comprising an NDL-Clade. One embodiment is directed to an NDL-Clade comprising a polypeptide or fragment, active fragment, or variant thereof, described herein. Another embodiment is directed to an NDL-Clade comprising a recombinant polypeptide or fragment, active fragment, or variant thereof, described herein. In some embodiments, the polypeptide or fragment, active fragment, or variant thereof is an endo-β-mannanase. In some embodiments, the recombinant polypeptide or fragment, active fragment, or variant thereof is an endo-β-mannanase. In one embodiment, the polypeptide or fragment, active fragment, or variant thereof described herein comprises Asn33-Asp-34-Leu35 (NDL), wherein the amino acid positions of the polypeptide are numbered by correspondence with the amino sequence set forth in SEQ ID NO:32 and are based on conserved linear sequence numbering. In some embodiments, the recombinant polypeptide or active fragment thereof of any of the above contains Asn33-Asp-34-Leu35 (NDL), wherein the amino acid positions of the polypeptide are numbered by correspondence with the amino sequence set forth in SEQ ID NO:32 and are based on conserved linear sequence numbering. In another embodiment, the NDL-Clade comprises a WXaKNDLXXAI motif at positions 30-38, wherein Xₐ is F or Y and X is any amino acid, wherein the amino acid positions of the polypeptide are numbered by correspondence with the amino sequence set forth in SEQ ID NO:32 and are based on conserved linear sequence numbering. In some embodiments, the polypeptide or fragment, active fragment, or variant thereof described herein contains a WXₐKNDLX_{b}X_{c}AI motif at positions 30-38, wherein Xₐ is F or Y, X_{b} is N, Y or A, and X_{c} is A or T, and wherein the amino acid positions of the polypeptide are numbered by correspondence with the amino sequence set forth in SEQ ID NO:32 and are based on conserved linear sequence numbering. In some embodiments, the recombinant polypeptide or fragment, active fragment, or variant thereof described herein contains a WXₐKNDLX_{b}X_{c}AI motif at positions 30-38, wherein Xₐ is F or Y, X_{b} is N, Y or A, and X_{c} is A or T, and wherein the amino acid positions of the polypeptide are numbered by correspondence with the amino sequence set forth in SEQ ID NO:32 and are based on conserved linear sequence numbering. In a further embodiment, the NDL-Clade comprises a L₂₆₂D₂₆₃XXXGPXGXL₂₇₂T₂₇₃, motif at positions 262-273, where X is any amino acid and wherein the amino acid positions of the polypeptide are numbered by correspondence with the amino sequence set forth in SEQ ID NO:32 and are based on the conserved linear sequence numbering. In yet a still further embodiment, the NDL-Clade comprises a L₂₆₂D₂₆₃M/LV/AT/AGPX₁GX₂L₂₇₂T₂₇₃ motif at positions 262-273, where X₁ is N, A or S and X₂ is S, T or N, and wherein the amino acid positions of the polypeptide are numbered by correspondence with the amino sequence set forth in SEQ ID NO:32 and are based on the conserved linear sequence numbering. One more embodiment is directed to an NDL-Clade 1 comprising a LDM/LATGPA/NGS/TLT motif at positions 262-273, wherein the amino acid positions of the polypeptide are numbered by correspondence with the amino sequence set forth in SEQ ID NO:32 and are based on the conserved linear sequence numbering. A still further embodiment is directed to an NDL-Clade 2 comprising a LDLA/VA/TGPS/NGNLT motif at positions 262-273, wherein the amino acid positions of the polypeptide are numbered by correspondence with the amino sequence set forth in SEQ ID NO:32 and are based on the conserved linear sequence numbering. Another embodiment is directed to an NDL-Clade 3 comprising a LDL/VS/AT/NGPSGNLT motif at positions 262-273, wherein the amino acid positions of the polypeptide are numbered by correspondence with the amino sequence set forth in SEQ ID NO:32 and are based on the conserved linear sequence numbering. In other embodiments, the polypeptide or recombinant polypeptide or fragment, active fragment, or variant thereof described herein has at least 70% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In some embodiments, the polypeptide or recombinant polypeptide or fragment, active fragment, or variant thereof described herein has at least 70% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, and 60. In some embodiments, the polypeptide or recombinant polypeptide or fragment, active fragment, or variant thereof described herein has mannanase activity, such as activity on locust bean gum galactomannan or konjac glucomannan. In some embodiments, the polypeptide or recombinant polypeptide or fragment, active fragment, or variant thereof described herein has mannanase activity in the presence of a surfactant. In some embodiments, the polypeptide or recombinant polypeptide or fragment, active fragment, or variant thereof described herein retains at least 70% of its maximal mannanase activity at a pH range of 4.5-9.0. In some embodiments, the polypeptide or recombinant polypeptide or fragment, active fragment, or variant thereof described herein retains at least 70% of its maximal mannanase activity at a temperature range of 40° C. to 70° C. In some embodiments, the polypeptide or recombinant polypeptide or fragment, active fragment, or variant thereof described herein has cleaning activity in a detergent composition. In some embodiments, the polypeptide or recombinant polypeptide or fragment, active fragment, or variant thereof described herein has mannanase activity in the presence of a protease. In some embodiments, the polypeptide or recombinant polypeptide or fragment, active fragment, or variant thereof described herein is capable of hydrolyzing a substrate selected from the group consisting of guar gum, locust bean gum, and combinations thereof. In some embodiments, the polypeptide or recombinant polypeptide or fragment, active fragment, or variant thereof described herein does not further comprise a carbohydrate-binding module.

Another embodiment is directed to cleaning compositions comprising at least one polypeptide of the preceding paragraph. Also provided by the present disclosure are cleaning compositions comprising at least one recombinant polypeptide of the preceding paragraph. In some embodiments, the composition further comprises a surfactant. In some preferred embodiments, the surfactant is an ionic surfactant. In some embodiments, the ionic surfactant is selected from the group consisting of an anionic surfactant, a cationic surfactant, a zwitterionic surfactant, and a combination thereof. In some preferred embodiments, the composition further comprises an enzyme selected from the group consisting of acyl transferases, amylases, alpha-amylases, beta-amylases, alpha-galactosidases, arabinases, arabinosidases, aryl esterases, beta-galactosidases, beta-glucanases, carrageenases, catalases, cellobiohydrolases, cellulases, chondroitinases, cutinases, endo-beta-1,4-glucanases, endo-beta-mannanases, exo-beta-mannanases, esterases, exo-mannanases, galactanases, glucoamylases, hemicellulases, hyaluronidases, keratinases, laccases, lactases, ligninases, lipases, lipolytic enzymes, lipoxygenases, mannanases, metalloproteases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, and combinations thereof. In some embodiments, the composition further comprises a protease and an amylase.

In some embodiments, the detergent is selected from the group consisting of a laundry detergent, a fabric softening detergent, a dishwashing detergent, and a hard-surface cleaning detergent. In some embodiments, the composition is a granular, powder, solid, bar, liquid, tablet, gel, paste, foam, sheet, or unit dose composition. In some embodiments, the detergent is in a form selected from the group consisting of a liquid, a powder, a granulated solid, and a tablet. The present disclosure further provides methods for hydrolyzing a mannan substrate present in a soil or stain on a surface, comprising: contacting the surface with the detergent composition to produce a clean surface. Also provided are methods of textile cleaning comprising: contacting a soiled textile with the detergent composition to produce a clean textile.

Moreover, the present disclosure provides nucleic acids or isolated nucleic acids encoding the polypeptide of the preceding paragraphs. Additionally, the present disclosure provides nucleic acids or isolated nucleic acids encoding the recombinant polypeptide of the preceding paragraphs. Further provided is an expression vector comprising a nucleic acid described herein operably linked to a regulatory sequence. Also provided is an expression vector comprising an isolated nucleic acid described herein in operable combination to a regulatory sequence. Additionally, host cells comprising an expression vector describe herein are provided. Another embodiment provides host cells comprising nucleic acids encoding a recombinant polypeptide described herein. In some embodiments, the host cell is a bacterial cell or a fungal cell.

The present disclosure further provides methods of producing an endo-β-mannanase of the present invention, comprising: culturing the host cell in a culture medium under suitable conditions to produce a culture comprising the endo-β-mannanase of the present invention. In some embodiments, the methods further comprise removing the host cells from the culture by centrifugation, and removing debris of less than 10 kDa by filtration to produce an endo-β-mannanase-enriched supernatant.

The present disclosure further provides methods for hydrolyzing a polysaccharide comprising: contacting a polysaccharide comprising mannose with the supernatant to produce oligosaccharides comprising mannose. In some embodiments, the polysaccharide is selected from the group consisting of mannan, glucomannan, galactomannan, galactoglucomannan, and combinations thereof.

These and other aspects of compositions and methods of the present invention will be apparent from the following description.

### DESCRIPTION OF THE DRAWINGS

FIG. 1 provides a plasmid map of p2JM-PspMan4.

FIGS. 2A-B show the cleaning performance of Paenibacillus and Bacillus spp. mannanases on Locust bean gum (CS-73) at pH 8, 20 minutes.

FIGS. 3A-C show the CLUSTAL W (1.83) multiple sequence alignment of mannanases including BciMan1, BciMan3, BciMan4, PamMan2, PpaMan2, PpoMan1, PpoMan2, PspMan4, PspMan5, PspMan9, and PtuMan2.

FIG. 4 shows a phylogenetic tree of mannanases including BciMan1, BciMan3, BciMan4, PamMan2, PpaMan2, PpoMan1, PpoMan2, PspMan4, PspMan5, PspMan9, and PtuMan2 showing the branching of the NDL-Clade mannanases from other mannanases and the differentiation of NDL-Clade 1 and NDL-Clade 2.

FIG. 5 shows the motif of the NDL-Clade mannanases at positions 30-38, using the conserved linear sequence numbering.

FIG. 6 shows the motif of the NDL-Clade mannanases, including the NDL-Clade 1 and NDL-Clade 2 mannanases, that is between the conserved Leu262-Asp263 (LD) and conserved Leu272-Thr273 (LT) residues, using the conserved linear sequence numbering.

FIG. 7 shows the potential structural consequences of motif changes found in the NDL-Clade mannanases. The closest known mannanase structure from Bacillus sp. JAMB-602 (1WKY) is shown in black while modelled structures of PspMan4, PspMan9 and PpaMan2 are shown in gray. The location of the deletion motif is highlighted by an arrow. The deletion motif is postulated to impact the structure of the loop in which it is located.

FIG. 8 shows the cleaning performance of PamMan3 and benchmank mannanases on Locust bean gum (CS-73) at pH 7.2, 30 minutes.

FIGS. 9A-9F show the alignment of multiple sequences of the mature forms of various mannanases that was created using CLUSTALW software.

FIG. 10 shows a phylogenetic tree for amino acid sequences of the mature forms of the various mannanases created using the Neighbor Joining method, and visualized using The Geneious Tree Builder program.

FIG. 11A-11C show the sequence alignment of the mature forms of the NDL-Clade mannanases that was created using CLUSTALW software.

Described herein are endo-β-mannanases from Paenibacillus or Bacillus spp, polynucleotides encoding such endo-β-mannanases, compositions containing such mannanases, and methods of use thereof. In one embodiment, the Paenibacillus and Bacillus spp. endo-β-mannanases described herein have glycosyl hydrolase activity in the presence of detergent compositions. This feature of the endo-β-mannanases described herein makes them well suited for use in a variety of cleaning and other industrial applications, for example, where the enzyme can hydrolyze mannans in the presence of surfactants and other components found in detergent compositions.

The following terms are defined for clarity. Terms and abbreviations not defined should be accorded their ordinary meaning as used in the art:

As used herein, a “mannan endo-1,4β-mannosidase,” “endo-1,4β-mannanase,” “endo-β-1,4-mannase,” “β-mannanase B,” “β-1,4-mannan 4-mannanohydrolase,” “endo-β-mannanase,” “β-D-mannanase,” “1,4β-D-mannan mannanohydrolase,” or “endo-β-mannanase” (EC 3.2.1.78) refers to an enzyme capable of the random hydrolysis of 1,4β-D-mannosidic linkages in mannans, galactomannans and glucomannans. Endo-1,4-β-mannanases are members of several families of glycosyl hydrolases, including GH26 and GH5. In particular, endo-β-mannanases constitute a group of polysaccharases that degrade mannans and denote enzymes that are capable of cleaving polyose chains containing mannose units (i.e., are capable of cleaving glycosidic bonds in mannans, glucomannans, galactomannans and galactoglucomannans). The “endo-β-mannanases” of the present disclosure may possess additional enzymatic activities (e.g., endo-1,4β-glucanase, 1,4-β-mannosidase, cellodextrinase activities, etc.).

As used herein, a “mannanase,” “mannosidic enzyme,” “mannolytic enzyme,” “mannanase enzyme,” “mannanase polypeptides,” or “mannanase proteins” refers to an enzyme, polypeptide, or protein exhibiting a mannan degrading capability. The mannanase enzyme may be, for example, an endo-β-mannanase, an exo-β-mannanase, or a glycosyl hydrolase. As used herein, mannanase activity may be determined according to any procedure known in the art (See, e.g., Lever, Anal. Biochem, 47:248, 1972; U.S. Pat. No. 6,602,842; and International Publication No. WO 95/35362A1).

As used herein, “mannans” are polysaccharides having a backbone composed of β-1,4-linked mannose; “glucomannans” are polysaccharides having a backbone of more or less regularly alternating β-1,4 linked mannose and glucose; “galactomannans” and “galactoglucomannans” are mannans and glucomannans with alpha-1,6 linked galactose sidebranches. These compounds may be acetylated. The degradation of galactomannans and galactoglucomannans is facilitated by full or partial removal of the galactose sidebranches. Further the degradation of the acetylated mannans, glucomannans, galactomannans and galactoglucomannans is facilitated by full or partial deacetylation. Acetyl groups can be removed by alkali or by mannan acetylesterases. The oligomers that are released from the mannanases or by a combination of mannanases and alpha-galactosidase and/or mannan acetyl esterases can be further degraded to release free maltose by β-mannosidase and/or β-glucosidase

As used herein, “catalytic activity” or “activity” describes quantitatively the conversion of a given substrate under defined reaction conditions. The term “residual activity” is defined as the ratio of the catalytic activity of the enzyme under a certain set of conditions to the catalytic activity under a different set of conditions. The term “specific activity” describes quantitatively the catalytic activity per amount of enzyme under defined reaction conditions.

As used herein, “pH-stability” describes the property of a protein to withstand a limited exposure to pH-values significantly deviating from the pH where its stability is optimal (e.g., more than one pH-unit above or below the pH-optimum, without losing its activity under conditions where its activity is measurable).

As used herein, the phrase “detergent stability” refers to the stability of a specified detergent composition component (such as a hydrolytic enzyme) in a detergent composition mixture.

As used herein, a “perhydrolase” is an enzyme capable of catalyzing a reaction that results in the formation of a peracid suitable for applications such as cleaning, bleaching, and disinfecting.

As used herein, the term “aqueous,” as used in the phrases “aqueous composition” and “aqueous environment,” refers to a composition that is made up of at least 50% water. An aqueous composition may contain at least 50% water, at least 60% water, at least 70% water, at least 80% water, at least 90% water, at least 95% water, at least 97% water, at least 99% water, or even at least 99% water.

As used herein, the term “surfactant” refers to any compound generally recognized in the art as having surface active qualities. Surfactants generally include anionic, cationic, nonionic, and zwitterionic compounds, which are further described, herein.

As used herein, “surface property” is used in reference to electrostatic charge, as well as properties such as the hydrophobicity and hydrophilicity exhibited by the surface of a protein.

The term “oxidation stability” refers to endo-β-mannanases of the present disclosure that retain a specified amount of enzymatic activity over a given period of time under conditions prevailing during the mannosidic, hydrolyzing, cleaning, or other process disclosed herein, for example while exposed to or contacted with bleaching agents or oxidizing agents. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after contact with a bleaching or oxidizing agent over a given time period, for example, at least about 1 minute, about 3 minutes, about 5 minutes, about 8 minutes, about 12 minutes, about 16 minutes, about 20 minutes, etc.

The term “chelator stability” refers to endo-β-mannanases of the present disclosure that retain a specified amount of enzymatic activity over a given period of time under conditions prevailing during the mannosidic, hydrolyzing, cleaning, or other process disclosed herein, for example while exposed to or contacted with chelating agents. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after contact with a chelating agent over a given time period, for example, at least about 10 minutes, about 20 minutes, about 40 minutes, about 60 minutes, about 100 minutes, etc.

The terms “thermal stability” and “thermostable” refer to endo-β-mannanases of the present disclosure that retain a specified amount of enzymatic activity after exposure to identified temperatures over a given period of time under conditions prevailing during the mannosidic, hydrolyzing, cleaning, or other process disclosed herein, for example, while exposed to altered temperatures. Altered temperatures include increased or decreased temperatures. In some embodiments, the endo-β-mannanases retain at least about 50%, about 60%, about 70%, about 75%, about 80%, about 85%, about 90%, about 92%, about 95%, about 96%, about 97%, about 98%, or about 99% endo-β-mannanase activity after exposure to altered temperatures over a given time period, for example, at least about 60 minutes, about 120 minutes, about 180 minutes, about 240 minutes, about 300 minutes, etc.

The term “cleaning activity” refers to the cleaning performance achieved by the endo-β-mannanase under conditions prevailing during the mannosidic, hydrolyzing, cleaning, or other process disclosed herein. In some embodiments, cleaning performance is determined by the application of various cleaning assays concerning enzyme sensitive stains arising from food products, household agents or personal care products. Some of these stains include, for example, ice cream, ketchup, BBQ sauce, mayonnaise, soups, chocolate milk, chocolate pudding, frozen desserts, shampoo, body lotion, sun protection products, toothpaste, locust bean gum, or guar gum as determined by various chromatographic, spectrophotometric or other quantitative methodologies after subjection of the stains to standard wash conditions. Exemplary assays include, but are not limited to those described in WO 99/34011, U.S. Pat. No. 6,605,458, and U.S. Pat. No. 6,566,114 (all of which are herein incorporated by reference), as well as those methods included in the Examples.

As used herein, the terms “clean surface” and “clean textile” refer to a surface or textile respectively that has a percent stain removal of at least 10%, preferably at least 15%, 20%, 25%, 30%, 35%, or 40% of a soiled surface or textile.

The term “cleaning effective amount” of an endo-β-mannanase refers to the quantity of endo-β-mannanase described herein that achieves a desired level of enzymatic activity in a specific cleaning composition. Such effective amounts are readily ascertained by one of ordinary skill in the art and are based on many factors, such as the particular endo-β-mannanase used, the cleaning application, the specific composition of the cleaning composition, and whether a liquid or dry (e.g., granular, bar, powder, solid, liquid, tablet, gel, paste, foam, sheet, or unit dose) composition is required, etc.

The term “cleaning adjunct materials”, as used herein, means any liquid, solid or gaseous material selected for the particular type of cleaning composition desired and the form of the product (e.g., liquid, granule, powder, bar, paste, spray, tablet, gel, unit dose, sheet, or foam composition), which materials are also preferably compatible with the endo-β-mannanase enzyme used in the composition. In some embodiments, granular compositions are in “compact” form, while in other embodiments, the liquid compositions are in a “concentrated” form.

As used herein, “cleaning compositions” and “cleaning formulations” refer to admixtures of chemical ingredients that find use in the removal of undesired compounds (e.g., soil or stains) from items to be cleaned, such as fabric, dishes, contact lenses, other solid surfaces, hair, skin, teeth, and the like. The compositions or formulations may be in the form of a liquid, gel, granule, powder, bar, paste, spray tablet, gel, unit dose, sheet, or foam, depending on the surface, item or fabric to be cleaned and the desired form of the composition or formulation.

As used herein, the terms “detergent composition” and “detergent formulation” refer to mixtures of chemical ingredients intended for use in a wash medium for the cleaning of soiled objects. Detergent compositions/formulations generally include at least one surfactant, and may optionally include hydrolytic enzymes, oxido-reductases, builders, bleaching agents, bleach activators, bluing agents and fluorescent dyes, caking inhibitors, masking agents, enzyme activators, antioxidants, and solubilizers.

As used herein, “dishwashing composition” refers to all forms of compositions for cleaning dishware, including cutlery, including but not limited to granular and liquid forms. In some embodiments, the dishwashing composition is an “automatic dishwashing” composition that finds use in automatic dish washing machines. It is not intended that the present disclosure be limited to any particular type or dishware composition. Indeed, the present disclosure finds use in cleaning dishware (e.g., dishes including, but not limited to plates, cups, glasses, bowls, etc.) and cutlery (e.g., utensils including, but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, the term “bleaching” refers to the treatment of a material (e.g., fabric, laundry, pulp, etc.) or surface for a sufficient length of time and under appropriate pH and temperature conditions to effect a brightening (i.e., whitening) and/or cleaning of the material. Examples of chemicals suitable for bleaching include but are not limited to ClO₂, H₂O₂, peracids, NO₂, etc.

As used herein, “wash performance” of a variant endo-β-mannanase refers to the contribution of a variant endo-β-mannanase to washing that provides additional cleaning performance to the detergent composition. Wash performance is compared under relevant washing conditions.

The term “relevant washing conditions” is used herein to indicate the conditions, particularly washing temperature, time, washing mechanics, sud concentration, type of detergent, and water hardness, actually used in households in a dish or laundry detergent market segment.

As used herein, the term “disinfecting” refers to the removal of contaminants from the surfaces, as well as the inhibition or killing of microbes on the surfaces of items. It is not intended that the present disclosure be limited to any particular surface, item, or contaminant(s) or microbes to be removed.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, a preferred filler salt is sodium sulfate.

The terms “textile” or “textile material” refer to woven fabrics, as well as staple fibers and filaments suitable for conversion to or use as yarns, woven, knit, and non-woven fabrics. The term encompasses yarns made from natural, as well as synthetic (e.g., manufactured) fibers.

A nucleic acid or polynucleotide is “isolated” when it is at least partially or completely separated from other components, including but not limited to for example, other proteins, nucleic acids, cells, etc. Similarly, a polypeptide, protein or peptide is “isolated” when it is at least partially or completely separated from other components, including but not limited to for example, other proteins, nucleic acids, cells, etc. On a molar basis, an isolated species is more abundant than are other species in a composition. For example, an isolated species may comprise at least about 60%, about 65%, about 70%, about 75%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

As used herein, a “polypeptide” refers to a molecule comprising a plurality of amino acids linked through peptide bonds. The terms “polypeptide,” “peptide,” and “protein” are used interchangeably. Proteins may optionally be modified (e.g., glycosylated, phosphorylated, acylated, farnesylated, prenylated, sulfonated, and the like) to add functionality. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single-stranded or double-stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type” and “native” refer to polypeptides or polynucleotides that are found in nature.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues and may be defined by their level of primary amino acid sequence homology/identity with a parent polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent polypeptide.

Sequence identity may be determined using known programs such as BLAST, ALIGN, and CLUSTAL using standard parameters. (See, e.g., Altschul et al. [1990] J. Mol. Biol. 215:403-410; Henikoff et al. [1989] Proc. Natl. Acad. Sci. USA 89:10915; Karin et al. [1993] Proc. Natl. Acad. Sci USA 90:5873; and Higgins et al. [1988] Gene 73:237-244). Software for performing BLAST analyses is publicly available through the National Center for Biotechnology Information. Databases may also be searched using FASTA (Pearson et al. Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution.

As used herein, a “variant polynucleotide” encodes a variant polypeptide, has a specified degree of homology/identity with a parent polynucleotide, or hybridizes under stringent conditions to a parent polynucleotide or the complement, thereof. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent polynucleotide. Methods for determining percent identity are known in the art and described immediately above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization” refers to the process by which a strand of nucleic acid joins with a complementary strand through base pairing, as known in the art.

As used herein, the phrase “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes, e.g.: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used). As used herein, stringent conditions are defined as 50° C. and 0.2×SSC (1×SSC=0.15 M NaCl, 0.015 M sodium citrate, pH 7.0).

The phrases “substantially similar” and “substantially identical” in the context of at least two nucleic acids or polypeptides means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the protein from the cell. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell. The terms “selectable marker” or “selectable gene product” as used herein refer to the use of a gene, which encodes an enzymatic activity that confers resistance to an antibiotic or drug upon the cell in which the selectable marker is expressed.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or pro-form of the protein variant, such variants, when expressed, are typically secreted from the host cell into the host cell medium.

The term “introduced” in the context of inserting a nucleic acid sequence into a cell, means transformation, transduction or transfection. Means of transformation include protoplast transformation, calcium chloride precipitation, electroporation, naked DNA, and the like as known in the art. (See, Chang and Cohen [1979] Mol. Gen. Genet. 168:111-115; Smith et al. Appl. Env. Microbiol. 51:634; and the review article by Ferrari et al., in Harwood, Bacillus, Plenum Publishing Corporation, pp. 57-72, 1989).

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, N Y 1991).

The singular terms “a,” “an,” and “the” include the plural reference unless the context clearly indicates otherwise.

As used herein in connection with a numerical value, the term “about” refers to a range of −10% to +10% of the numerical value. For instance, the phrase a “pH value of about 6” refers to pH values of from 5.4 to 6.6.

Headings are provided for convenience and should not be construed as limitations. The description included under one heading may apply to the specification as a whole.

Paenibacillus and Bacillus Spp. Polypeptides

One embodiment is directed to an NDL-Clade comprising a polypeptide or fragment, active fragment, or variant thereof, described herein. Another embodiment is directed to an NDL-Clade comprising a recombinant polypeptide or fragment, active fragment, or variant thereof, described herein. In some embodiments, the polypeptide or recombinant polypeptide or fragment, active fragment, or variant thereof, is an endo-β-mannanase. In some embodiments, the polypeptide or recombinant polypeptide or fragment, active fragment, or variant thereof, described herein contains Asn33-Asp-34-Leu35 (NDL), wherein the amino acid positions of the polypeptide are numbered by correspondence with the amino sequence set forth in SEQ ID NO:32 and are based on conserved linear sequence numbering.

In one aspect, a composition or method described herein comprise a polypeptide or recombinant polypeptide or fragment, active fragment, or variant thereof, in the NDL-Clade. In another aspect, a polypeptide or recombinant polypeptide or fragment, active fragment, or variant thereof described herein is used in the methods or compositions described herein.

In one aspect, the present compositions and methods provide a recombinant endo-β-mannanase polypeptide or fragment, active fragment, or variant thereof, in the NDL-Clade. In yet a further aspect, the present compositions and methods comprise a recombinant endo-β-mannanase polypeptide or fragment, active fragment, or variant thereof, in the NDL-Clade. In yet still further aspect, the present compositions and methods comprise a endo-β-mannanase polypeptide or fragment, active fragment, or variant thereof, in the NDL-Clade. A still further aspect is directed to a polypeptide or recombinant polypeptide endo-β-mannanase. or fragment, active fragment, or variant thereof, in the NDL-Clade. One embodiment is directed to an NDL-Clade of endo-β-mannanase polypeptides. Another embodiment is directed to an NDL-Clade 1 of endo-β-mannanase polypeptides. Yet another embodiment is directed to an NDL-Clade 2 of endo-β-mannanase polypeptides. A still further embodiment is directed to an NDL-Clade 3 of endo-β-mannanase polypeptides.

In some embodiments, the NDL-Clade comprises an Asn33-Asp-34-Leu35, wherein the amino acid positions of the polypeptide are numbered by correspondence with the amino sequence set forth in SEQ ID NO:32 and are based on the conserved linear sequence numbering. In another embodiment, the NDL-Clade comprises a WXaKNDLXXAI motif at positions 30-38, wherein Xₐ is F or Y and X is any amino acid, wherein the amino acid positions of the polypeptide are numbered by correspondence with the amino sequence set forth in SEQ ID NO:32 and are based on the conserved linear sequence numbering. In some embodiments, the NDL-Clade comprises a WXₐKNDLX_{b}X_{c}AI motif at positions 30-38, wherein Xₐ is F or Y, X_{b} is N, Y or A, and X_{c} is A or T, wherein the amino acid positions of the polypeptide are numbered by correspondence with the amino sequence set forth in SEQ ID NO:32 and are based on the conserved linear sequence numbering.

In a further embodiment, the NDL-Clade comprises a L₂₆₂D₂₆₃XXXGPXGXL₂₇₂T₂₇₃, motif at positions 262-273, where X is any amino acid and wherein the amino acid positions of the polypeptide are numbered by correspondence with the amino sequence set forth in SEQ ID NO:32 and are based on the conserved linear sequence numbering. In yet a still further embodiment, the NDL-Clade comprises a L₂₆₂D₂₆₃M/LWAT/AGPX₁GX₂L₂₇₂T₂₇₃ motif at positions 262-273, where X₁ is N, A or S and X₂ is S, T or N, and wherein the amino acid positions of the polypeptide are numbered by correspondence with the amino sequence set forth in SEQ ID NO:32 and are based on the conserved linear sequence numbering. In some embodiments, NDL-Clade 1 comprises a LDM/LATGPN/AGS/TLT motif at positions 262-273, wherein the amino acid positions of the polypeptide are numbered by correspondence with the amino sequence set forth in SEQ ID NO:32 and are based on the conserved linear sequence numbering. In some embodiments, NDL-Clade 2 comprises an LDLA/VA/TGPS/NGNLT motif at positions 262-273, wherein the amino acid positions of the polypeptide are numbered by correspondence with the amino sequence set forth in SEQ ID NO:32 and are based on the conserved linear sequence numbering. In yet other embodiments, NDL-Clade 3 comprises an LDL/VS/AT/NGPSGNLT motif at positions 262-273, wherein the amino acid positions of the polypeptide are numbered by correspondence with the amino sequence set forth in SEQ ID NO:32 and are based on the conserved linear sequence numbering.

In one aspect, the present compositions and methods provide a Paenibacillus or Bacillus spp. endo-β-mannanase polypeptide or fragment, active fragment, or variant thereof described herein. Exemplary Paenibacillus or Bacillus spp. polypeptides include BciMan1(SEQ ID NO:2) isolated from B. circulans K−1, BciMan3 (SEQ ID NO:4) isolated from B. circulans 196, BciMan4 (SEQ ID NO:6) isolated from B. circulans CGMCC1554, PpoMan1 (SEQ ID NO:8) isolated from Paenibacillus polymyxa E681, PpoMan2 (SEQ ID NO:10) isolated from Paenibacillus polymyxa SC2, PspMan4 (SEQ ID NO:12) isolated from Paenibacillus sp. A1, PspMan5 (SEQ ID NO:14) isolated from Paenibacillus sp. CH-3, PamMan2 (precursor protein is SEQ ID NO:16 and mature protein is SEQ ID NO:17) isolated from Paenibacillus amylolyticus, PamMan3 (SEQ ID NO:63) isolated from Paenibacillus sp. N02/strain, PpaMan2 (precursor protein is SEQ ID NO:19) isolated from Paenibacillus pabuli, PspMan9 (precursor protein is SEQ ID NO:21) isolated from Paenibacillus sp. FeL05, and PtuMan2 (precursor protein is SEQ ID NO:23 and mature protein is SEQ ID NO:24) isolated from Paenibacillus tundrae. These and other isolated PspMan4 polypeptides are encompassed by the present compositions and methods.

Another embodiment is directed to polypeptide or a recombinant polypeptide or fragment, active fragment, or variant thereof described herein, comprising an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. Another embodiment is directed a recombinant polypeptide or fragment, active fragment, or variant thereof described herein comprising an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In some embodiments, the invention is a recombinant polypeptide or fragment, active fragment, or variant thereof of any of the above described embodiments, comprising an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, and 60. In yet a further embodiment, an NDL-Clade polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In a still further embodiment, an NDL-Clade recombinant polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, an NDL-Clade 1 recombinant polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet another embodiment, an NDL-Clade 1 polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In an even further embodiment, an NDL-Clade 2 polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 4, 8, 10, 30, 31, 32, 42, 43, 44, 46, 47, 48, 72, and 73. In yet still a further embodiment, an NDL-Clade 2 recombinant polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 4, 8, 10, 30, 31, 32, 42, 43, 44, 46, 47, 48, 72, and 73. In still yet an even further embodiment, an NDL-Clade 3 polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 74 and 81. In yet an even still further embodiment, an NDL-Clade 3 recombinant polypeptide or fragment, active fragment, or variant thereof further comprises an amino acid sequence having at least 60%, 65%, 70%, 75%, 80%, 85%, 90%, 91%, 92%, 93%, 94%, 95%, 96%, 97%, 98%, 99% or greater identity to an amino acid sequence selected from SEQ ID NO: 74 and 81.

In other embodiments, the polypeptide or recombinant polypeptide or fragment, active fragment, or variant thereof of any of the above has at least 70% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In yet a further embodiment, an NDL-Clade polypeptide or recombinant polypeptide or fragment, active fragment, or variant thereof comprises an amino acid sequence having at least 70% identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, an NDL-Clade 1 polypeptide or recombinant polypeptide or fragment, active fragment, or variant thereof further comprises an amino acid sequence having at least 70% identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet still a further embodiment, an NDL-Clade 2 polypeptide or recombinant polypeptide or fragment, active fragment, or variant thereof comprises an amino acid sequence having at least 70% identity to an amino acid sequence selected from SEQ ID NO: 4, 8, 10, 30, 31, 32, 42, 43, 44, 46, 47, 48, 72, and 73. In yet an even still further embodiment, an NDL-Clade 3 polypeptide or recombinant polypeptide or fragment, active fragment, or variant thereof comprises an amino acid sequence having at least 70% identity to an amino acid sequence selected from SEQ ID NO: 74 and 81.

In other embodiments, the polypeptide or recombinant polypeptide or fragment, active fragment, or variant thereof of any of the above has at least 80% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In yet a further embodiment, an NDL-Clade polypeptide or recombinant polypeptide or fragment, active fragment, or variant thereof further comprises an amino acid sequence having at least 80% identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, an NDL-Clade 1 polypeptide or recombinant polypeptide or fragment, active fragment, or variant thereof further comprises an amino acid sequence having at least 80% identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet still a further embodiment, an NDL-Clade 2 polypeptide or recombinant polypeptide or fragment, active fragment, or variant thereof further comprises an amino acid sequence having at least 80% identity to an amino acid sequence selected from SEQ ID NO: 4, 8, 10, 30, 31, 32, 42, 43, 44, 46, 47, 48, 72, and 73. In yet an even still further embodiment, an NDL-Clade 3 polypeptide or recombinant polypeptide or fragment, active fragment, or variant thereof further comprises an amino acid sequence having at least 80% identity to an amino acid sequence selected from SEQ ID NO: 74 and 81.

In other embodiments, the polypeptide or recombinant polypeptide or fragment, active fragment, or variant thereof of any of the above has at least 90% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In yet a further embodiment, an NDL-Clade polypeptide or recombinant polypeptide or fragment, active fragment, or variant thereof further comprises an amino acid sequence having at least 90% identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, an NDL-Clade 1 polypeptide or recombinant polypeptide or fragment, active fragment, or variant thereof further comprises an amino acid sequence having at least 90% identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet still a further embodiment, an NDL-Clade 2 polypeptide or recombinant polypeptide or fragment, active fragment, or variant thereof further comprises an amino acid sequence having at least 90% identity to an amino acid sequence selected from SEQ ID NO: 4, 8, 10, 30, 31, 32, 42, 43, 44, 46, 47, 48, 72, and 73. In yet an even still further embodiment, an NDL-Clade 3 polypeptide or recombinant polypeptide or fragment, active fragment, or variant thereof further comprises an amino acid sequence having at least 90% identity to an amino acid sequence selected from SEQ ID NO: 74 and 81.

In other embodiments, the polypeptide or recombinant polypeptide or fragment, active fragment, or variant thereof of any of the above has at least 95% identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In yet a further embodiment, an NDL-Clade polypeptide or recombinant polypeptide or fragment, active fragment, or variant thereof further comprises an amino acid sequence having at least 95% identity to an amino acid sequence selected from SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, an NDL-Clade 1 polypeptide or recombinant polypeptide or fragment, active fragment, or variant thereof further comprises an amino acid sequence having at least 95% identity to an amino acid sequence selected from SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet still a further embodiment, an NDL-Clade 2 polypeptide or recombinant polypeptide or fragment, active fragment, or variant thereof further comprises an amino acid sequence having at least 95% identity to an amino acid sequence selected from SEQ ID NO: 4, 8, 10, 30, 31, 32, 42, 43, 44, 46, 47, 48, 72, and 73. In yet an even still further embodiment, an NDL-Clade 3 polypeptide or recombinant polypeptide or fragment, active fragment, or variant thereof further comprises an amino acid sequence having at least 95% identity to an amino acid sequence selected from SEQ ID NO: 74 and 81.

In some embodiments, the invention is a recombinant polypeptide or fragment, active fragment, or variant thereof of any of the above described embodiments, comprising an amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, and 60. In yet a still further embodiment, the invention is a polypeptide or recombinant polypeptide or fragment, active fragment, or variant thereof of any of the above described embodiments, comprising an amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In yet further embodiments, the invention is an NDL-Clade polypeptide or recombinant polypeptide or fragment, active fragment, or variant thereof comprising an amino acid sequence selected from the group consisting of SEQ ID NO: 4, 6, 8, 10, 12, 14, 16, 17, 19, 21, 23, 24, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In another embodiment, the invention is an NDL-Clade 1 polypeptide or recombinant polypeptide or fragment, active fragment, or variant thereof comprising an amino acid sequence selected from the group consisting of SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71. In yet still a further embodiment, the invention is an NDL-Clade 2 polypeptide or recombinant polypeptide or fragment, active fragment, or variant thereof comprising an amino acid sequence selected from the group consisting of SEQ ID NO: 4, 8, 10, 30, 31, 32, 42, 43, 44, 46, 47, 48, 72, and 73. In yet an even still further embodiment, the invention is an NDL-Clade 3 polypeptide or recombinant polypeptide or fragment, active fragment, or variant thereof comprising an amino acid sequence selected from the group consisting of SEQ ID NO: 74 and 81.

Sequence identity can be determined by amino acid sequence alignment, e.g., using a program such as BLAST, ALIGN, or CLUSTAL, as described herein. In some embodiments, the polypeptides of the present invention are isolated polypeptides.

In one embodiment, the invention is a polypeptide or fragment, active fragment, or variant thereof of any of the above described embodiments, wherein the polypeptide has mannanase activity. In some embodiments, the invention is a recombinant polypeptide or fragment, active fragment, or variant thereof of any of the above described embodiments, wherein the polypeptide has mannanase activity. In some embodiments, the mannanase activity is activity on mannan gum. In some embodiments, the mannanase activity is activity on locust bean gum galactomannan. In some embodiments, the mannanase activity is activity on konjac glucomannan.

In one embodiment, the invention is a polypeptide or fragment, active fragment, or variant thereof of any of the above described embodiments, wherein the mannanase activity is in the presence of a surfactant. In some embodiments, the invention is a recombinant polypeptide or an active fragment thereof of any of the above described embodiments, wherein the mannanase activity is in the presence of a surfactant.

In some embodiments, the invention is a polypeptide or fragment, active fragment, or variant thereof of any of the above described embodiments, wherein the polypeptide retains at least 70% of its maximal protease activity at a pH range of 4.5-9.0. In some embodiments, the invention is a recombinant polypeptide or fragment, active fragment, or variant thereof of any of the above described embodiments, wherein the polypeptide retains at least 70% of its maximal protease activity at a pH range of 4.5-9.0. In some embodiments, the invention is a polypeptide or fragment, active fragment, or variant thereof of any of the above described embodiments, wherein the polypeptide retains at least 70% of its maximal protease activity at a pH range of 5.5-8.5. In some embodiments, the invention is a recombinant polypeptide or fragment, active fragment, or variant thereof any of the above described embodiments, wherein the polypeptide retains at least 70% of its maximal protease activity at a pH range of 5.5-8.5. In some embodiments, the invention is a polypeptide or fragment, active fragment, or variant thereof any of the above described embodiments, wherein the polypeptide retains at least 70% of its maximal protease activity at a pH range of 6.0-7.5. In some embodiments, the invention is a recombinant polypeptide or fragment, active fragment, or variant thereof any of the above described embodiments, wherein the polypeptide retains at least 70% of its maximal protease activity at a pH range of 6.0-7.5. In some embodiments, the invention is a polypeptide or fragment, active fragment, or variant thereof of any of the above described embodiments, wherein the polypeptide retains at least 70% of its maximal protease activity at a pH above 3.0, 3.5, 4.0 or 4.5. In some embodiments, the invention is a recombinant polypeptide or fragment, active fragment, or variant thereof of any of the above described embodiments, wherein the polypeptide retains at least 70% of its maximal protease activity at a pH above 3.0, 3.5, 4.0 or 4.5. In some embodiments, the invention is a polypeptide or fragment, active fragment, or variant thereof any of the above described embodiments, wherein the polypeptide retains at least 70% of its maximal protease activity at a pH below 10.0, 9.5, or 9.0. In some embodiments, the invention is a recombinant polypeptide or fragment, active fragment, or variant thereof any of the above described embodiments, wherein the polypeptide retains at least 70% of its maximal protease activity at a pH below 10.0, 9.5, or 9.0.

In some embodiments, the invention is a polypeptide or fragment, active fragment, or variant thereof of any of the above described embodiments, wherein the polypeptide retains at least 70% of its maximal protease activity at a temperature range of 40° C. to 70° C. In some embodiments, the invention is a polypeptide or fragment, active fragment, or variant thereof any of the above described embodiments, wherein the polypeptide retains at least 70% of its maximal protease activity at a temperature range of 45° C. to 65° C. In some embodiments, the invention is a polypeptide or fragment, active fragment, or variant thereof any of the above described embodiments, wherein the polypeptide retains at least 70% of its maximal protease activity at a temperature range of 50° C. to 60° C. In some embodiments, the invention is a polypeptide or fragment, active fragment, or variant thereof any of the above described embodiments, wherein the polypeptide retains at least 70% of its maximal protease activity at a temperature above 20° C., 25° C., 30° C., 35° C., or 40° C. In some embodiments, the invention is a polypeptide or fragment, active fragment, or variant thereof any of the above described embodiments, wherein the polypeptide retains at least 70% of its maximal protease activity at a temperature below 90° C., 85° C., 80° C., 75° C., or 70° C.

In some embodiments, the invention is a recombinant polypeptide or fragment, active fragment, or variant thereof of any of the above described embodiments, wherein the polypeptide retains at least 70% of its maximal protease activity at a temperature range of 40° C. to 70° C. In some embodiments, the invention is a recombinant polypeptide or fragment, active fragment, or variant thereof any of the above described embodiments, wherein the polypeptide retains at least 70% of its maximal protease activity at a temperature range of 45° C. to 65° C. In some embodiments, the invention is a recombinant polypeptide or fragment, active fragment, or variant thereof any of the above described embodiments, wherein the polypeptide retains at least 70% of its maximal protease activity at a temperature range of 50° C. to 60° C. In some embodiments, the invention is a recombinant polypeptide or fragment, active fragment, or variant thereof any of the above described embodiments, wherein the polypeptide retains at least 70% of its maximal protease activity at a temperature above 20° C., 25° C., 30° C., 35° C., or 40° C. In some embodiments, the invention is a recombinant polypeptide or fragment, active fragment, or variant thereof any of the above described embodiments, wherein the polypeptide retains at least 70% of its maximal protease activity at a temperature below 90° C., 85° C., 80° C., 75° C., or 70° C.

In some embodiments, the invention is a polypeptide or fragment, active fragment, or variant thereof of any of the above described embodiments, wherein the polypeptide has cleaning activity in a detergent composition. In some embodiments, the invention is a recombinant polypeptide or fragment, active fragment, or variant thereof of any of the above described embodiments, wherein the polypeptide has cleaning activity in a detergent composition.

In some embodiments, the invention is a polypeptide or fragment, active fragment, or variant thereof of any of the above described embodiments, wherein the polypeptide has cleaning activity in a detergent composition. In some embodiments, the invention is a polypeptide or fragment, active fragment, or variant thereof of any of the above described embodiments, wherein the polypeptide has mannanase activity in the presence of a protease. In some embodiments, the invention is a polypeptide or fragment, active fragment, or variant thereof of any of the above described embodiments, wherein the polypeptide is capable of hydrolyzing a substrate selected from the group consisting of guar gum, locust bean gum, and combinations thereof.

In some embodiments, the invention is a recombinant polypeptide or fragment, active fragment, or variant thereof of any of the above described embodiments, wherein the polypeptide has cleaning activity in a detergent composition. In some embodiments, the invention is a recombinant polypeptide or fragment, active fragment, or variant thereof of any of the above described embodiments, wherein the polypeptide has mannanase activity in the presence of a protease. In some embodiments, the invention is a recombinant polypeptide or fragment, active fragment, or variant thereof of any of the above described embodiments, wherein the polypeptide is capable of hydrolyzing a substrate selected from the group consisting of guar gum, locust bean gum, and combinations thereof.

In some embodiments, the invention is a polypeptide or fragment, active fragment, or variant thereof of any of the above described embodiments, wherein the polypeptide does not further comprise a carbohydrate-binding module. In some embodiments, the invention is a recombinant polypeptide or fragment, active fragment, or variant thereof of any of the above described embodiments, wherein the polypeptide does not further comprise a carbohydrate-binding module.

In certain embodiments, the polypeptides of the present invention are produced recombinantly, while in others the polypeptides of the present invention are produced synthetically, or are purified from a native source.

In certain other embodiments, the polypeptide of the present invention includes substitutions that do not substantially affect the structure and/or function of the polypeptide. Exemplary substitutions are conservative mutations, as summarized in Table I.

TABLE I
Amino Acid Substitutions

| Original Residue | Code | Acceptable Substitutions |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, |
| | | Trp, D-Trp, Trans-3,4, or 5-phenylproline, |
| | | cis-3,4, or 5-phenylproline |
| Proline | P | D-Pro, L-I-thioazolidine-4- carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant a polypeptide of the present invention, and then expressing the variant polypeptide in an organism. Substitutions involving non-naturally occurring amino acids or chemical modifications to amino acids are generally made by chemically modifying a recombinant a polypeptide of the present invention after it has been synthesized by an organism.

In some embodiments, variant isolated polypeptides of the present invention are substantially identical to SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, or 60, meaning that they do not include amino acid substitutions, insertions, or deletions that do not significantly affect the structure, function, or expression of the polypeptide. In some embodiments, variant isolated polypeptides of the present invention are substantially identical to SEQ ID NO: SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81, meaning that they do not include amino acid substitutions, insertions, or deletions that do not significantly affect the structure, function, or expression of the polypeptide. In some embodiments, variant isolated polypeptides of the present invention are substantially identical to SEQ ID NO: SEQ ID NO: SEQ ID NO: 6, 12, 14, 16, 17, 19, 21, 23, 24, 34, 35, 36, 38, 39, 40, 50, 51, 52, 54, 55, 56, 58, 59, 60, 62, 63, 65, 66, 67, 68, 69, 70, and 71, meaning that they do not include amino acid substitutions, insertions, or deletions that do not significantly affect the structure, function, or expression of the polypeptide. In some embodiments, variant isolated polypeptides of the present invention are substantially identical to SEQ ID NO: 4, 8, 10, 30, 31, 32, 42, 43, 44, 46, 47, 48, 72, and 73, meaning that they do not include amino acid substitutions, insertions, or deletions that do not significantly affect the structure, function, or expression of the polypeptide. In some embodiments, variant isolated polypeptides of the present invention are substantially identical to SEQ ID NO: 74 and 81, meaning that they do not include amino acid substitutions, insertions, or deletions that do not significantly affect the structure, function, or expression of the polypeptide. Such variant isolated a polypeptide of the present inventions include those designed only to circumvent the present description.

In some embodiments, a polypeptide of the present invention (including a variant thereof) has 1,4-β-D-mannosidic hydrolase activity, which includes mannanase, endo-1,4-β-D-mannanase, exo-1,4-β-D-mannanasegalactomannanase, and/or glucomannanase activity. 1,4β-D-mannosidic hydrolase activity can be determined and measured using the assays described herein, or by other assays known in the art. In some embodiments, a polypeptide of the present invention has activity in the presence of a detergent composition.

A polypeptide of the present invention include fragments of “full-length” polypeptides that retain 1,4-β-D-mannosidic hydrolase activity. Such fragments preferably retain the active site of the full-length polypeptides but may have deletions of non-critical amino acid residues. The activity of fragments can readily be determined using the assays described, herein, or by other assays known in the art. In some embodiments, the fragments of a polypeptide of the present invention retain 1,4-β-D-mannosidic hydrolase activity in the presence of a detergent composition.

In some embodiments, a polypeptide of the present invention's amino acid sequences and derivatives are produced as a N- and/or C-terminal fusion protein, for example to aid in extraction, detection and/or purification and/or to add functional properties to a polypeptide of the present invention. Examples of fusion protein partners include, but are not limited to, glutathione-S-transferase (GST), 6×His, GAL4 (DNA binding and/or transcriptional activation domains), FLAG, MYC, BCE103 (WO 2010/044786), or other tags well known to anyone skilled in the art. In some embodiments, a proteolytic cleavage site is provided between the fusion protein partner and the protein sequence of interest to allow removal of fusion protein sequences. Preferably, the fusion protein does not hinder the activity of a polypeptide of the present invention.

In some embodiments, a polypeptide of the present invention is fused to a functional domain including a leader peptide, propeptide, one or more binding domain (modules) and/or catalytic domain. Suitable binding domains include, but are not limited to, carbohydrate-binding modules (e.g., CBM) of various specificities, providing increased affinity to carbohydrate components present during the application of a polypeptide of the present invention. As described herein, the CBM and catalytic domain of a polypeptide of the present invention are operably linked.

A carbohydrate-binding module (CBM) is defined as a contiguous amino acid sequence within a carbohydrate-active enzyme with a discreet fold having carbohydrate-binding activity. A few exceptions are CBMs in cellulosomal scaffoldin proteins and rare instances of independent putative CBMs. The requirement of CBMs existing as modules within larger enzymes sets this class of carbohydrate-binding protein apart from other non-catalytic sugar binding proteins such as lectins and sugar transport proteins. CBMs were previously classified as cellulose-binding domains (CBDs) based on the initial discovery of several modules that bound cellulose (Tomme et al., Eur J Biochem, 170:575-581, 1988; and Gilkes et al., J Biol Chem, 263:10401-10407, 1988). However, additional modules in carbohydrate-active enzymes are continually being found that bind carbohydrates other than cellulose yet otherwise meet the CBM criteria, hence the need to reclassify these polypeptides using more inclusive terminology. Previous classification of cellulose-binding domains was based on amino acid similarity. Groupings of CBDs were called “Types” and numbered with roman numerals (e.g. Type I or Type II CBDs). In keeping with the glycoside hydrolase classification, these groupings are now called families and numbered with Arabic numerals. Families 1 to 13 are the same as Types I to XIII (Tomme et al., in Enzymatic Degradation of Insoluble Polysaccharides (Saddler, J. N. & Penner, M., eds.), Cellulose-binding domains: classification and properties. pp. 142-163, American Chemical Society, Washington, 1995). A detailed review on the structure and binding modes of CBMs can be found in (Boraston et al., Biochem J, 382:769-81, 2004). The family classification of CBMs is expected to: aid in the identification of CBMs, in some cases, predict binding specificity, aid in identifying functional residues, reveal evolutionary relationships and possibly be predictive of polypeptide folds. Because the fold of proteins is better conserved than their sequences, some of the CBM families can be grouped into superfamilies or clans. The current CBM families are 1-63. CBMs/CBDs have also been found in algae, e.g., the red alga Porphyra purpurea as a non-hydrolytic polysaccharide-binding protein. However, most of the CBDs are from cellullases and xylanases. CBDs are found at the N- and C-termini of proteins or are internal. Enzyme hybrids are known in the art (See e.g., WO 90/00609 and WO 95/16782) and may be prepared by transforming into a host cell a DNA construct comprising at least a fragment of DNA encoding the cellulose-binding domain ligated, with or without a linker, to a DNA sequence encoding a disclosed polypeptide of the present invention and growing the host cell to express the fused gene. Enzyme hybrids may be described by the following formula:

CBM-MR-X or X-MR-CBM

In the above formula, the CBM is the N-terminal or the C-terminal region of an amino acid sequence corresponding to at least the carbohydrate-binding module; MR is the middle region (the linker), and may be a bond, or a short linking group preferably of from about 2 to about 100 carbon atoms, more preferably of from 2 to 40 carbon atoms; or is preferably from about 2 to about 100 amino acids, more preferably from 2 to 40 amino acids; and X is an N-terminal or C-terminal region of a polypeptide of the present invention having mannanase catalytic activity. In addition, a mannanase may contain more than one CBM or other module(s)/domain(s) of non-glycolytic function. The terms “module” and “domain” are used interchangeably in the present disclosure.

Suitable enzymatically active domains possess an activity that supports the action of a polypeptide of the present invention in producing the desired product. Non-limiting examples of catalytic domains include: cellulases, hemicellulases such as xylanase, exo-mannanases, glucanases, arabinases, galactosidases, pectinases, and/or other activities such as proteases, lipases, acid phosphatases and/or others or functional fragments thereof. Fusion proteins are optionally linked to a polypeptide of the present invention through a linker sequence that simply joins a polypeptide of the present invention and the fusion domain without significantly affecting the properties of either component, or the linker optionally has a functional importance for the intended application.

Alternatively, polypeptides of the present invention described herein are used in conjunction with one or more additional proteins of interest. Non-limiting examples of proteins of interest include: acyl transferases, amylases, alpha-amylases, beta-amylases, alpha-galactosidases, arabinases, arabinosidases, aryl esterases, beta-galactosidases, beta-glucanases, carrageenases, catalases, cellobiohydrolases, cellulases, chondroitinases, cutinases, endo-beta-1,4-glucanases, endo-beta-mannanases, exo-beta-mannanases, esterases, exo-mannanases, galactanases, glucoamylases, hemicellulases, hyaluronidases, keratinases, laccases, lactases, ligninases, lipases, lipolytic enzymes, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases and/or other enzymes.

In other embodiments, a polypeptide of the present invention is fused to a signal peptide for directing the extracellular secretion of a polypeptide of the present invention. For example, in certain embodiments, the signal peptide is the native signal peptide of a polypeptide of the present invention. In other embodiments, the signal peptide is a non-native signal peptide such as the B. subtilis AprE signal peptide. In some embodiments, a polypeptide of the present invention has an N-terminal extension of Ala-Gly-Lys between the mature form and the signal peptide.

In some embodiments, a polypeptide of the present invention is expressed in a heterologous organism, i.e., an organism other than Paenibacillus and Bacillus spp. Exemplary heterologous organisms are Gram(+) bacteria such as Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus; Gram(−) bacteria such as Escherichia coli; yeast such as Saccharomyces spp. or Schizosaccharomyces spp., e.g. Saccharomyces cerevisiae; and filamentous fungi such as Aspergillus spp., e.g., Aspergillus oryzae or Aspergillus niger, and Trichoderma reesei. Methods from transforming nucleic acids into these organisms are well known in the art. A suitable procedure for transformation of Aspergillus host cells is described in EP 238 023.

In particular embodiments, a polypeptide of the present invention is expressed in a heterologous organism as a secreted polypeptide, in which case, the compositions and method encompass a method for expressing a polypeptide of the present invention as a secreted polypeptide in a heterologous organism.

### Polynucleotides of the Present Invention

Another aspect disclosed herein is a polynucleotide that encodes a polypeptide of the present invention (including variants and fragments thereof). In one aspect, the polynucleotide is provided in the context of an expression vector for directing the expression of a polypeptide of the present invention in a heterologous organism, such as those identified, herein. The polynucleotide that encodes a polypeptide of the present invention may be operably-linked to regulatory elements (e.g., a promoter, terminator, enhancer, and the like) to assist in expressing the encoded polypeptides.

Exemplary polynucleotide sequences encoding a polypeptide of the present invention has the nucleotide sequence of SEQ ID NO: 1, 3, 5, 7, 9, 11, 13, 15, 18, 20, 22, 25, 29, 33, 37, 41, 45, 49, 53, 57, 61 or 64. Exemplary polynucleotide sequences encoding a polypeptide of the present invention has the nucleotide sequence of SEQ ID NO: 1, 3, 5, 7, 9, 11, 13, 15, 18, 20, 22, 25, 29, 33, 37, 41, 45, 49, 53, or 57. Similar, including substantially identical, polynucleotides encoding a polypeptide of the present invention and variants may occur in nature, e.g., in other strains or isolates of B. agaradhaerens. In view of the degeneracy of the genetic code, it will be appreciated that polynucleotides having different nucleotide sequences may encode the same a polypeptide of the present inventions, variants, or fragments.

In some embodiments, polynucleotides encoding a polypeptide of the present invention have a specified degree of amino acid sequence identity to the exemplified polynucleotide encoding a polypeptide of the present invention, e.g., at least 60%, 65%, 70%, 75%, 80%, 85%, 90%, 91%, 92%, 93%, 94%, 95%, 96%, 97%, 98%, 99% or greater identity to the amino acid sequence selected from the group consisting of SEQ ID NO:2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81. In some embodiments, polynucleotides encoding a polypeptide of the present invention have a specified degree of amino acid sequence identity to the exemplified polynucleotide encoding a polypeptide of the present invention, e.g., at least 60%, 65%, 70%, 75%, 80%, 85%, 90%, 91%, 92%, 93%, 94%, 95%, 96%, 97%, 98%, 99% or greater identity to the amino acid sequence selected from the group consisting of SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, and 60. Homology can be determined by amino acid sequence alignment, e.g., using a program such as BLAST, ALIGN, or CLUSTAL, as described herein.

In some embodiments, polynucleotides can have a specified degree of nucleotide sequence identity to the exemplified polynucleotides of the present invention, e.g., at least 60%, 65%, 70%, 75%, 80%, 85%, 90%, 91%, 92%, 93%, 94%, 95%, 96%, 97%, 98%, 99% or greater identity to the nucleotide sequence selected from the group consisting of SEQ ID NO: 1, 3, 5, 7, 9, 11, 13, 15, 18, 20, 22, 25, 29, 33, 37, 41, 45, 49, 53, 57, 61 or 64. In some embodiments, polynucleotides can have a specified degree of nucleotide sequence identity to the exemplified polynucleotides of the present invention, e.g., at least 60%, 65%, 70%, 75%, 80%, 85%, 90%, 91%, 92%, 93%, 94%, 95%, 96%, 97%, 98%, 99% or greater identity to the nucleotide sequence selected from the group consisting of SEQ ID NO: 1, 3, 5, 7, 9, 11, 13, 15, 18, 20, 22, 25, 29, 33, 37, 41, 45, 49, 53, or 57. Homology can be determined by amino acid sequence alignment, e.g., using a program such as BLAST, ALIGN, or CLUSTAL, as described herein.

In some embodiments, the polynucleotide that encodes a polypeptide of the present invention is fused in frame behind (i.e., downstream of) a coding sequence for a signal peptide for directing the extracellular secretion of a polypeptide of the present invention. Heterologous signal sequences include those from bacterial cellulase genes. Expression vectors may be provided in a heterologous host cell suitable for expressing a polypeptide of the present invention, or suitable for propagating the expression vector prior to introducing it into a suitable host cell.

In some embodiments, polynucleotides encoding a polypeptide of the present invention hybridize to the exemplary polynucleotide of SEQ ID NO: 1, 3, 5, 7, 9, 11, 13, 15, 18, 20, 22, 25, 29, 33, 37, 41, 45, 49, 53, 57, 61 or 64 (or the complement thereof) under specified hybridization conditions. In some embodiments, polynucleotides encoding a polypeptide of the present invention hybridize to the exemplary polynucleotide of SEQ ID NO: 1, 3, 5, 7, 9, 11, 13, 15, 18, 20, 22, 25, 29, 33, 37, 41, 45, 49, 53, or 57 (or the complement thereof) under specified hybridization conditions. Exemplary conditions are stringent condition and highly stringent conditions, which are described, herein.

A polynucleotide of the present invention may be naturally occurring or synthetic (i.e., man-made), and may be codon-optimized for expression in a different host, mutated to introduce cloning sites, or otherwise altered to add functionality.

### Vectors and Host Cells

In order to produce a disclosed a polypeptide of the present invention, the DNA encoding the polypeptide can be chemically synthesized from published sequences or obtained directly from host cells harboring the gene (e.g., by cDNA library screening or PCR amplification). In some embodiments, a polynucleotide of the present invention is included in an expression cassette and/or cloned into a suitable expression vector by standard molecular cloning techniques. Such expression cassettes or vectors contain sequences that assist initiation and termination of transcription (e.g., promoters and terminators), and generally contain a selectable marker.

The expression cassette or vector is introduced in a suitable expression host cell, which then expresses the corresponding polynucleotide of the present invention. Particularly suitable expression hosts are bacterial expression host genera including Escherichia (e.g., Escherichia coli), Pseudomonas (e.g., P. fluorescens or P. stutzerei), Proteus (e.g., Proteus mirabilis), Ralstonia (e.g., Ralstonia eutropha), Streptomyces, Staphylococcus (e.g., S. carnosus), Lactococcus (e.g., L. lactis), or Bacillus (subtilis, megaterium, licheniformis, etc.). Also particularly suitable are yeast expression hosts such as Saccharomyces cerevisiae, Schizosaccharomyces pombe, Yarrowia lipolytica, Hansenula polymorpha, Kluyveromyces lactis or Pichia pastoris. Especially suited are fungal expression hosts such as Aspergillus niger, Chrysosporium lucknowense, Aspergillus (e.g., A. oryzae, A. niger, A. nidulans, etc.) or Trichoderma reesei. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing a polypeptide of the present invention.

Promoters and/or signal sequences associated with secreted proteins in a particular host of interest are candidates for use in the heterologous production and secretion of endo-β-mannanases in that host or in other hosts. As an example, in filamentous fungal systems, the promoters that drive the genes for cellobiohydrolase I (cbh1), glucoamylase A (glaA), TAKA-amylase (amyA), xylanase (ex1A), the gpd-promoter cbh1, cbhll, endoglucanase genes EGI-EGV, Ce161B, Ce174A, egl1-eg15, gpd promoter, Pgk1, pki1, EF-1alpha, tef1, cDNA1 and hex1 are particularly suitable and can be derived from a number of different organisms (e.g., A. niger, T reesei, A. oryzae, A. awamori and A. nidulans). In some embodiments, a polynucleotide of the present invention is recombinantly associated with a polynucleotide encoding a suitable homologous or heterologous signal sequence that leads to secretion of a polypeptide of the present invention into the extracellular (or periplasmic) space, thereby allowing direct detection of enzyme activity in the cell supernatant (or periplasmic space or lysate). Particularly suitable signal sequences for Escherichia coli, other Gram negative bacteria and other organisms known in the art include those that drive expression of the HlyA, DsbA, Pbp, PhoA, PelB, OmpA, OmpT or M13 phage Gill genes. For Bacillus subtilis, Gram-positive organisms and other organisms known in the art, particularly suitable signal sequences further include those that drive expression of the AprE, NprB, Mpr, AmyA, AmyE, Blac, SacB, and for S. cerevisiae or other yeast, include the killer toxin, Bar1, Suc2, Mating factor alpha, Inu1A or Ggplp signal sequence. Signal sequences can be cleaved by a number of signal peptidases, thus removing them from the rest of the expressed protein. In some embodiments, the rest of the polypeptide is expressed alone or as a fusion with other peptides, tags or proteins located at the N- or C-terminus (e.g., 6×His, HA or FLAG tags). Suitable fusions include tags, peptides or proteins that facilitate affinity purification or detection (e.g., BCE103, 6×His, HA, chitin binding protein, thioredoxin or FLAG tags), as well as those that facilitate expression, secretion or processing of the target endo-β-mannanase. Suitable processing sites include enterokinase, STE13, Kex2 or other protease cleavage sites for cleavage in vivo or in vitro.

Polynucleotides of the present invention can be introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

Alternatively, a polypeptide of the present invention can be expressed intracellularly. Optionally, after intracellular expression of the enzyme variants, or secretion into the periplasmic space using signal sequences such as those mentioned above, a permeabilisation or lysis step can be used to release the polypeptide into the supernatant. The disruption of the membrane barrier is effected by the use of mechanical means such as ultrasonic waves, pressure treatment (French press), cavitation or the use of membrane-digesting enzymes such as lysozyme or enzyme mixtures. As a further alternative, the polynucleotides encoding the polypeptide can be expressed by use of a suitable cell-free expression system. In cell-free systems, the polynucleotide of interest is typically transcribed with the assistance of a promoter, but ligation to form a circular expression vector is optional. In other embodiments, RNA is exogenously added or generated without transcription and translated in cell free systems.

The polypeptides of the present invention disclosed herein may have enzymatic activity over a broad range of pH conditions. In certain embodiments the disclosed polypeptides of the present invention have enzymatic activity from about pH 4.0 to about pH 11.0, or from about pH 4.5 to about pH 11.0. In preferred embodiments, the polypeptides have substantial enzymatic activity, for example, at least 50%, 60%, 70%, 80%, 90%, 95%, or 100% activity from about pH 4.0 to 11.0, pH 4.5 to 11.0, pH 4.5 to 9.0, pH 5.5 to 8.5, or pH 6.0 to 7.5. It should be noted that the pH values described herein may vary by ±0.2. For example a pH value of about 8.0 could vary from pH 7.8 to pH 8.2.

The polypeptides of the present invention disclosed herein may have enzymatic activity over a wide range of temperatures, e.g., from about 20° C. or lower to 90° C., 30° C. to 80° C., 40° C. to 70° C., 45° C. to 65° C., or 50° C. to 60° C. In certain embodiments, the polypeptides have substantial enzymatic activity, for example, at least 50%, 60%, 70%, 80%, 90%, 95%, or 100% activity at a temperature range of about 20° C. or lower to 90° C., 30° C. to 80° C., 40° C. to 70° C., 45° C. to 65° C., or 50° C. to 60° C. It should be noted that the temperature values described herein may vary by ±0.2° C. For example a temperature of about 50° C. could vary from 49.8° C. to 50.2° C.

### Detergent Compositions Comprising a Polypeptide of the Present Invention

An aspect of the compositions and methods disclosed herein is a detergent composition comprising an isolated a polypeptide of the present invention (including variants or fragments, thereof) and methods for using such compositions in cleaning applications. Cleaning applications include, but are not limited to, laundry or textile cleaning, laundry or textile softening, dishwashing (manual and automatic), stain pre-treatment, and the like. Particular applications are those where mannans (e.g., locust bean gum, guar gum, etc.) are a component of the soils or stains to be removed. Detergent compositions typically include an effective amount of any of the polypeptides of the present inventions described herein, e.g., at least 0.0001 weight percent, from about 0.0001 to about 1, from about 0.001 to about 0.5, from about 0.01 to about 0.1 weight percent, or even from about 0.1 to about 1 weight percent, or more. An effective amount of a polypeptide of the present invention in the detergent composition results in the polypeptide of the present invention having enzymatic activity sufficient to hydrolyze a mannan-containing substrate, such as locust bean gum, guar gum, or combinations thereof.

Additionally, detergent compositions having a concentration from about 0.4 g/L to about 2.2 g/L, from about 0.4 g/L to about 2.0 g/L, from about 0.4 g/L to about 1.7 g/L, from about 0.4 g/L to about 1.5 g/L, from about 0.4 g/L to about 1 g/L, from about 0.4 g/L to about 0.8 g/L, or from about 0.4 g/L to about 0.5 g/L may be mixed with an effective amount of an isolated a polypeptide of the present invention. The detergent composition may also be present at a concentration of about 0.4 ml/L to about 2.6 ml/L, from about 0.4 ml/L to about 2.0 ml/L, from about 0.4 ml/L to about 1.5 m/L, from about 0.4 ml/L to about 1 ml/L, from about 0.4 ml/L to about 0.8 ml/L, or from about 0.4 ml/L to about 0.5 ml/L.

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. In the exemplified detergent compositions, the enzymes levels are expressed by pure enzyme by weight of the total composition and unless otherwise specified, the detergent ingredients are expressed by weight of the total compositions.

In some embodiments, the detergent composition comprises one or more surfactants, which may be non-ionic, semi-polar, anionic, cationic, zwitterionic, or combinations and mixtures thereof. The surfactants are typically present at a level of from about 0.1% to 60% by weight. Exemplary surfactants include but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof.

Anionic surfactants that may be used with the detergent compositions described herein include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. It may also contain 0-40% of nonionic surfactant such as alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO 92/06154), and combinations and mixtures thereof.

Nonionic surfactants that may be used with the detergent compositions described herein include but are not limited to polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

The detergent compositions disclosed herein may have mixtures that include, but are not limited to 5-15% anionic surfactants, <5% nonionic surfactants, cationic surfactants, phosphonates, soap, enzymes, perfume, butylphenyl methylptopionate, geraniol, zeolite, polycarboxylates, hexyl cinnamal, limonene, cationic surfactants, citronellol, and benzisothiazolinone.

Detergent compositions may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, an optical brightener, a fabric conditioner, and a perfume. The detergent compositions may also include enzymes, including but not limited to proteases, amylases, cellulases, lipases, pectin degrading enzymes, xyloglucanases, or additional carboxylic ester hydrolases. The pH of the detergent compositions should be neutral to basic, as described herein.

In some embodiments incorporating at least one builder, the detergent compositions comprise at least about 1%, from about 3% to about 60% or even from about 5% to about 40% builder by weight of the cleaning composition. Builders may include, but are not limited to, the alkali metals, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, the various alkali metals, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP 2 100 949).

As indicated herein, in some embodiments, the cleaning compositions described herein further comprise adjunct materials including, but not limited to surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See, e.g., U.S. Pat. Nos. 6,610,642; 6,605,458; 5,705,464; 5,710,115; 5,698,504; 5,695,679; 5,686,014; and 5,646,101; all of which are incorporated herein by reference). Embodiments of specific cleaning composition materials are exemplified in detail below. In embodiments in which the cleaning adjunct materials are not compatible with the polypeptides of the present invention in the cleaning compositions, suitable methods of keeping the cleaning adjunct materials and the endo-β-mannanase(s) separated (i.e., not in contact with each other), until combination of the two components is appropriate, are used. Such separation methods include any suitable method known in the art (e.g., gelcaps, encapsulation, tablets, physical separation, etc.).

The cleaning compositions described herein are advantageously employed for example, in laundry applications, hard surface cleaning, dishwashing applications, as well as cosmetic applications such as dentures, teeth, hair, and skin. In addition, due to the unique advantages of increased effectiveness in lower temperature solutions, the polypeptides described herein are ideally suited for laundry and fabric softening applications. Furthermore, the polypeptides of the present invention may find use in granular and liquid compositions.

A polypeptide or isolated polypeptide described herein may also find use cleaning in additive products. In some embodiments, low temperature solution cleaning applications find use. In some embodiments, the present disclosure provides cleaning additive products including at least one disclosed a polypeptide of the present invention is ideally suited for inclusion in a wash process when additional bleaching effectiveness is desired. Such instances include, but are not limited to low temperature solution cleaning applications. In some embodiments, the additive product is in its simplest form, one or more endo-β-mannanases. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present disclosure, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to various salts of sulfate, carbonate, and silicate as well as talc, clay, and the like. Suitable filler or carrier materials for liquid compositions include, but are not limited to water or low molecular weight primary and secondary alcohols including polyols and diols. Examples of such alcohols include, but are not limited to methanol, ethanol, propanol, and isopropanol. In some embodiments, the compositions contain from about 5% to about 90% of such materials. Acidic fillers find use to reduce pH. Alternatively, in some embodiments, the cleaning additive includes adjunct ingredients, as described more fully below.

In one embodiment, the present cleaning compositions or cleaning additives contain an effective amount of at least one polypeptide described herein, optionally in combination with other endo-β-mannanases and/or additional enzymes. In certain embodiments, the additional enzymes include, but are not limited to, at least one enzyme selected from acyl transferases, amylases, alpha-amylases, beta-amylases, alpha-galactosidases, arabinases, arabinosidases, aryl esterases, beta-galactosidases, beta-glucanases, carrageenases, catalases, cellobiohydrolases, cellulases, chondroitinases, cutinases, endo-beta-1, 4-glucanases, endo-beta-mannanases, exo-beta-mannanases, esterases, exo-mannanases, galactanases, glucoamylases, hemicellulases, hyaluronidases, keratinases, laccases, lactases, ligninases, lipases, lipolytic enzymes, lipoxygenases, mannanases, metalloproteases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, and mixtures thereof.

The required level of enzyme is achieved by the addition of one or more disclosed a polypeptide of the present invention. Typically the present cleaning compositions will comprise at least about 0.0001 weight percent, from about 0.0001 to about 10, from about 0.001 to about 1, or even from about 0.01 to about 0.1 weight percent of at least one of the disclosed a polypeptide of the present inventions.

The cleaning compositions herein are typically formulated such that, during use in aqueous cleaning operations, the wash water will have a pH of from about 3.0 to about 11. Liquid product formulations are typically formulated to have a neat pH from about 5.0 to about 9.0. Granular laundry products are typically formulated to have a pH from about 8.0 to about 11.0. Techniques for controlling pH at recommended usage levels include the use of buffers, alkalis, acids, etc., and are well known to those skilled in the art.

Suitable low pH cleaning compositions typically have a neat pH of from about 3.0 to about 5.0 or even from about 3.5 to about 4.5. Low pH cleaning compositions are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine, or hydrochloric acid, to provide such cleaning composition with a neat pH of from about 3.0 to about 5.0. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of the composition wherein the solvent is distilled water. In these embodiments, all pH measurements are taken at 20° C., unless otherwise indicated.

Suitable high pH cleaning compositions typically have a neat pH of from about 9.0 to about 11.0, or even a net pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the a polypeptide of the present invention is employed in a granular composition or liquid, it is desirable for the a polypeptide of the present invention to be in the form of an encapsulated particle to protect the a polypeptide of the present invention from other components of the granular composition during storage. In addition, encapsulation is also a means of controlling the availability of the a polypeptide of the present invention during the cleaning process. In some embodiments, encapsulation enhances the performance of the a polypeptide of the present invention and/or additional enzymes. In this regard, the a polypeptide of the present inventions of the present disclosure are encapsulated with any suitable encapsulating material known in the art. In some embodiments, the encapsulating material typically encapsulates at least part of the catalyst for the a polypeptide of the present inventions described herein. Typically, the encapsulating material is water-soluble and/or water-dispersible. In some embodiments, the encapsulating material has a glass transition temperature (Tg) of 0° C. or higher. Glass transition temperature is described in more detail in the PCT application WO 97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See, e.g., EP 0 922 499; U.S. Pat. Nos. 4,977,252; 5,354,559; and 5,935,826). In some embodiments, the encapsulating material is a microsphere made from plastic such as thermoplastics, acrylonitrile, methacrylonitrile, polyacrylonitrile, polymethacrylonitrile, and mixtures thereof commercially available microspheres that find use include, but are not limited to those supplied by EXPANCEL® (Stockviksverken, Sweden), and PM 6545, PM 6550, PM 7220, PM 7228, EXTENDOSPHERES®, LUXSIL®, Q-CEL®, and SPHERICEL® (PQ Corp., Valley Forge, Pa.).

The term “granular composition” refers to a conglomeration of discrete solid, macroscopic particles. Powders are a special class of granular material due to their small particle size, which makes them more cohesive and more easily suspended.

In using detergent compositions that include a polypeptide of the present invention in cleaning applications, the fabrics, textiles, dishes, or other surfaces to be cleaned are incubated in the presence of a detergent composition having a polypeptide of the present invention for a time sufficient to allow the polypeptide to hydrolyze mannan substrates including, but not limited to, locust bean gum, guar gum, and combinations thereof present in soil or stains, and then typically rinsed with water or another aqueous solvent to remove the detergent composition along with hydrolyzed mannans.

As described herein, a polypeptide of the present inventions find particular use in the cleaning industry, including, but not limited to laundry and dish detergents. These applications place enzymes under various environmental stresses. A polypeptide of the present inventions may provide advantages over many currently used enzymes, due to their stability under various conditions.

Indeed, there are a variety of wash conditions including varying detergent formulations, wash water volumes, wash water temperatures, and lengths of wash time, to which endo-β-mannanases involved in washing are exposed. In addition, detergent formulations used in different geographical areas have different concentrations of their relevant components present in the wash water. For example, European detergents typically have about 4500-5000 ppm of detergent components in the wash water, while Japanese detergents typically have approximately 667 ppm of detergent components in the wash water. In North America, particularly the United States, detergents typically have about 975 ppm of detergent components present in the wash water.

A low detergent concentration system includes detergents where less than about 800 ppm of the detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of the detergent components are present in the wash water. North American detergents are generally considered to be medium detergent concentration systems as they have approximately 975 ppm of detergent components present in the wash water. Brazil typically has approximately 1500 ppm of detergent components present in the wash water.

A high detergent concentration system includes detergents where greater than about 2000 ppm of the detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. As mentioned above, Brazil typically has approximately 1500 ppm of detergent components present in the wash water. However, other high suds phosphate builder detergent geographies, not limited to other Latin American countries, may have high detergent concentration systems up to about 6000 ppm of detergent components present in the wash water.

In light of the foregoing, it is evident that concentrations of detergent compositions in typical wash solutions throughout the world varies from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 975 ppm of detergent within the wash solution about 62.79 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 30° C. (e.g., about 20° C.), whereas the temperature of wash water in Europe is typically between about 30 and about 60° C. (e.g., about 40° C.). Accordingly, in certain embodiments, the detergent compositions described herein may be utilized at temperature from about 10° C. to about 60° C., or from about 20° C. to about 60° C., or from about 30° C. to about 60° C., or from about 40° C. to about 60° C., as well as all other combinations within the range of about 40° C. to about 55° C., and all ranges within 10° C. to 60° C. However, in the interest of saving energy, many consumers are switching to using cold water washing. In addition, in some further regions, cold water is typically used for laundry, as well as dish washing applications. In some embodiments, the “cold water washing” of the present disclosure utilizes washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

TABLE II
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Accordingly, in some embodiments, the present disclosure provides a polypeptide of the present inventions that show surprising wash performance in at least one set of wash conditions (e.g., water temperature, water hardness, and/or detergent concentration). In some embodiments, a polypeptide of the present inventions are comparable in wash performance to other endo-β-mannanases. In some embodiments, a polypeptide of the present inventions exhibit enhanced wash performance as compared to endo-β-mannanases currently commercially available. Thus, in some preferred embodiments, the a polypeptide of the present inventions provided herein exhibit enhanced oxidative stability, enhanced thermal stability, enhanced cleaning capabilities under various conditions, and/or enhanced chelator stability. In addition, a polypeptide of the present inventions may find use in cleaning compositions that do not include detergents, again either alone or in combination with builders and stabilizers.

In some embodiments of the present disclosure, the cleaning compositions comprise at least one a polypeptide of the present invention of the present disclosure at a level from about 0.00001% to about 10% by weight of the composition and the balance (e.g., about 99.999% to about 90.0%) comprising cleaning adjunct materials by weight of composition. In other aspects of the present disclosure, the cleaning compositions comprises at least one a polypeptide of the present invention at a level of about 0.0001% to about 10%, about 0.001% to about 5%, about 0.001% to about 2%, about 0.005% to about 0.5% by weight of the composition and the balance of the cleaning composition (e.g., about 99.9999% to about 90.0%, about 99.999% to about 98%, about 99.995% to about 99.5% by weight) comprising cleaning adjunct materials.

In addition to the polypeptide of the present inventions provided herein, any other suitable endo-β-mannanases find use in the compositions described herein either alone or in combination with a polypeptide described herein. Suitable endo-β-mannanases include, but are not limited to, endo-β-mannanases of the GH26 family of glycosyl hydrolases, endo-β-mannanases of the GH5 family of glycosyl hydrolases, acidic endo-β-mannanases, neutral endo-β-mannanases, and alkaline endo-β-mannanases. Examples of alkaline endo-β-mannanases include those described in U.S. Pat. Nos. 6,060,299, 6,566,114, and 6,602,842; WO 9535362A1, WO 9964573A1, WO9964619A1, and WO2015022428. Additionally, suitable endo-β-mannanases include, but are not limited to those of animal, plant, fungal, or bacterial origin. Chemically or genetically modified mutants are encompassed by the present disclosure.

Examples of useful endo-β-mannanases include Bacillus endo-β-mannanases such as B. subtilis endo-β-mannanase (See, e.g., U.S. Pat. No. 6,060,299, and WO 9964573A1), B. sp. 1633 endo-β-mannanase (See, e.g., U.S. Pat. No. 6,566,114 and WO9964619A1), Bacillus sp. AAI12 endo-β-mannanase (See, e.g., U.S. Pat. No. 6,566,114 and WO9964619A1), B. sp. AA349 endo-β-mannanase (See, e.g., U.S. Pat. No. 6,566,114 and WO9964619A1), B. agaradhaerens NCIMB 40482 endo-β-mannanase (See, e.g., U.S. Pat. No. 6,566,114 and WO9964619A1), B. halodurans endo-β-mannanase, B. clausii endo-β-mannanase (See, e.g., U.S. Pat. No. 6,566,114 and WO9964619A1), B. licheniformis endo-β-mannanase (See, e.g., U.S. Pat. No. 6,566,114 and WO9964619A1), Humicola endo-β-mannanases such as H. insolens endo-β-mannanase (See, e.g., U.S. Pat. No. 6,566,114 and WO9964619A1), and Caldocellulosiruptor endo-β-mannanases such as C. sp. endo-β-mannanase (See, e.g., U.S. Pat. No. 6,566,114 and WO9964619A1).

Furthermore, a number of identified mannanases (i.e., endo-β-mannanases and exo-β-mannanases) find use in some embodiments of the present disclosure, including but not limited to Agaricus bisporus mannanase (See, Tang et al., [2001] Appl. Environ. Microbiol. 67: 2298-2303), Aspergillus tamarii mannanase (See, Civas et al., [1984] Biochem. J. 219: 857-863), Aspergillus aculeatus mannanase (See, Christgau et al., [1994] Biochem. Mol. Biol. Int. 33: 917-925), Aspergillus awamori mannanase (See, Setati et al., [2001] Protein Express Purif. 21: 105-114), Aspergillus fumigatus mannanase (See, Puchart et al., [2004] Biochimica et biophysica Acta. 1674: 239-250), Aspergillus niger mannanase (See, Ademark et al., [1998] J. Biotechnol. 63: 199-210), Aspergillus oryzae NRRL mannanase (See, Regalado et al., [2000] J. Sci. Food Agric. 80: 1343-1350), Aspergillus sulphureus mannanase (See, Chen et al., [2007] J. Biotechnol. 128(3): 452-461), Aspergillus terrus mannanase (See, Huang et al., [2007] Wei Sheng Wu Xue Bao. 47(2): 280-284), Paenibacillus and Bacillus spp. mannanase (See, U.S. Pat. No. 6,376,445.), Bacillus AM001 mannanase (See, Akino et al., [1989] Arch. Microbiol. 152: 10-15), Bacillus brevis mannanase (See, Araujo and Ward, [1990] J. Appl. Bacteriol. 68: 253-261), Bacillus circulans K−1 mannanase (See, Yoshida et al., [1998] Biosci. Biotechnol. Biochem. 62(3): 514-520), Bacillus polymyxa mannanase (See, Araujo and Ward, [1990] J. Appl. Bacteriol. 68: 253-261), Bacillus sp JAMB-750 mannanase (See, Hatada et al., [2005] Extremophiles. 9: 497-500), Bacillus sp. M50 mannanase (See, Chen et al., [2000] Wei Sheng Wu Xue Bao. 40: 62-68), Bacillus sp. N 16-5 mannanase (See, Yanhe et al., [2004] Extremophiles 8: 447-454), Bacillus stearothermophilus mannanase (See, Talbot and Sygusch, Appl. Environ. Microbiol. 56: 3505-3510), Bacillus subtilis mannanase (See, Mendoza et al., [1994] World J. Microbiol. Biotechnol. 10: 51-54), Bacillus subtilis B36 mannanase (Li et al., [2006] Z. Naturforsch (C). 61: 840-846), Bacillus subtilis BM9602 mannanase (See, Cui et al., [1999] Wei Sheng Wu Xue Bao. 39(1): 60-63), Bacillus subtilis SA-22 mannanase (See, Sun et al., [2003] Sheng Wu Gong Cheng Xue Bao. 19(3): 327-330), Bacillus subtilis168 mannanase (See, Helow and Khattab, [1996] Acta Microbiol. Immunol. Hung. 43: 289-299), Bacteroides ovatus mannanase (See, Gherardini et al., [1987] J. Bacteriol. 169: 2038-2043), Bacteroides ruminicola mannanase (See, Matsushita et al., [1991] J. Bacteriol. 173: 6919-6926), Caldibacillus cellulovorans mannanase (See, Sunna et al., [2000] Appl. Environ. Microbiol. 66: 664-670), Caldocellulosiruptor saccharolyticus mannanase (See, Morris et al., [1995] Appl. Environ. Microbiol. 61: 2262-2269), Caldocellum saccharolyticum mannanase (See, Bicho et al., [1991] Appl. Microbiol. Biotechnol. 36: 337-343), Cellulomonas fimi mannanase (See, Stoll et al., [1999] Appl. Environ. Microbiol. 65(6):2598-2605), Clostridium butyricum/beijerinckii mannanase (See, Nakajima and Matsuura, [1997] Biosci. Biotechnol. Biochem. 61: 1739-1742), Clostridium cellulolyticum mannanase (See, Perret et al., [2004] Biotechnol. Appl. Biochem. 40: 255-259), Clostridium tertium mannanase (See, Kataoka and Tokiwa, [1998] J. Appl. Microbiol. 84: 357-367), Clostridium thermocellum mannanase (See, Halstead et al., [1999] Microbiol. 145: 3101-3108), Dictyoglomus thermophilum mannanase (See, Gibbs et al., [1999] Curr. Microbiol. 39(6): 351-357), Flavobacterium sp mannanase (See, Zakaria et al., [1998] Biosci. Biotechnol. Biochem. 62: 655-660), Gastropoda pulmonata mannanase (See, Charrier and Rouland, [2001] J. Expt. Zool. 290: 125-135), Littorina brevicula mannanase (See, Yamamura et al., [1996] Biosci. Biotechnol. Biochem. 60: 674-676), Lycopersicon esculentum mannanase (See, Filichkin et al., [2000] Plant Physiol. 134:1080-1087), Paenibacillus curdlanolyticus mannanase (See, Pason and Ratanakhanokchai, [2006] Appl. Environ. Microbiol. 72: 2483-2490), Paenibacillus polymyxa mannanase (See, Han et al., [2006] Appl. Microbiol Biotechnol. 73(3): 618-630), Phanerochaete chrysosporium mannanase (See, Wymelenberg et al., [2005] J. Biotechnol. 118: 17-34), Piromyces sp. mannanase (See, Fanutti et al., [1995] J. Biol. Chem. 270(49): 29314-29322), Pomacea insulars mannanase (See, Yamamura et al., [1993] Biosci. Biotechnol. Biochem. 7: 1316-1319), Pseudomonas fluorescens subsp. Cellulose mannanase (See, Braithwaite et al., [1995] Biochem J. 305: 1005-1010), Rhodothermus marinus mannanase (See, Politz et al., [2000] Appl. Microbiol. Biotechnol. 53 (6): 715-721), Sclerotium rolfsii mannanase (See, Sachslehner et al., [2000] J. Biotechnol. 80:127-134), Streptomyces galbus mannanase (See, Kansoh and Nagieb, [2004] Anton. van. Leeuwonhoek. 85: 103-114), Streptomyces lividans mannanase (See, Arcand et al., [1993] J. Biochem. 290: 857-863), Thermoanaerobacterium Polysaccharolyticum mannanase (See, Cann et al., [1999] J. Bacteriol. 181: 1643-1651), Thermomonospora fusca mannanase (See, Hilge et al., [1998] Structure 6: 1433-1444), Thermotoga maritima mannanase (See, Parker et al., [2001] Biotechnol. Bioeng. 75(3): 322-333), Thermotoga neapolitana mannanase (See, Duffaud et al., [1997] Appl. Environ. Microbiol. 63: 169-177), Trichoderma harzanium strain T4 mannanase (See, Franco et al., [2004] Biotechnol Appl. Biochem. 40: 255-259), Trichoderma reesei mannanase (See, Stalbrand et al., [1993] J. Biotechnol. 29: 229-242), and Vibrio sp. mannanase (See, Tamaru et al., [1997] J. Ferment. Bioeng. 83: 201-205).

Additional suitable endo-β-mannanases include commercially available endo-β-mannanases such as HEMICELL® (Chemgen); GAMANASE® and MANNAWAY®, (Novozymes A/S, Denmark); PURABRITE™ and MANNASTAR™ (Genencor, A Danisco Division, Palo Alto, Calif.); and PYROLASE® 160 and PYROLASE® 200 (Diversa).

In some embodiments of the present disclosure, the cleaning compositions of the present disclosure further comprise endo-β-mannanases at a level from about 0.00001% to about 10% of additional endo-β-mannanase by weight of the composition and the balance of cleaning adjunct materials by weight of composition. In other aspects of the present disclosure, the cleaning compositions of the present disclosure also comprise endo-β-mannanases at a level of about 0.0001% to about 10%, about 0.001% to about 5%, about 0.001% to about 2%, about 0.005% to about 0.5% endo-β-mannanase by weight of the composition.

In some embodiments of the present disclosure, any suitable protease may be used. Suitable proteases include those of animal, vegetable or microbial origin. In some embodiments, chemically or genetically modified mutants are included. In some embodiments, the protease is a serine protease, preferably an alkaline microbial protease or a trypsin-like protease. Various proteases are described in PCT applications WO 95/23221 and WO 92/21760; U.S. Pat. Publication No. 2008/0090747; and U.S. Pat. Nos. 5,801,039; 5,340,735; 5,500,364; 5,855,625; U.S. RE 34,606; 5,955,340; 5,700,676; 6,312,936; 6,482,628; and various other patents. In some further embodiments, metalloproteases find use in the present disclosure, including but not limited to the neutral metalloprotease described in PCT application WO 07/044993. Commercially available protease enzymes that find use in the present invention include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280), EFFECTENZ™ proteases (e.g. P1000, P1050, P2000), EXCELLENZ™ proteases (e.g. P1000), ULTIMASE®, and PURAFAST™ (DuPont); ALCALASE®, SAVINASE®, PRIMASE®, DURAZYM™, POLARZYME®, OVOZYME®, KANNASE®, LIQUANASE®, NEUTRASE®, RELASE® and ESPERASE® (Novozymes); BLAP™ and BLAP™ variants (Henkel Kommanditgesellschaft auf Aktien, Duesseldorf, Germany), and KAP (B. alkalophilus subtilisin; Kao Corp., Tokyo, Japan).

In some embodiments of the present disclosure, any suitable amylase may be used. In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present disclosure include, but are not limited to α-amylases obtained from B. licheniformis (See, e.g., GB 1,296,839). Commercially available amylases that find use in the present disclosure include, but are not limited to DURAIVIYL®, TERMAMYL®, FUNGAMYL®, STAINZYME®, STAINZYME PLUS®, STAINZYME ULTRA®, and BAN™ (Novozymes A/S, Denmark), as well as PURASTAR®, POWERASE™, RAPIDASE, and MAXAMYL® P (Genencor, A Danisco Division, Palo Alto, Calif.).

In some embodiments of the present disclosure, the disclosed cleaning compositions further comprise amylases at a level from about 0.00001% to about 10% of additional amylase by weight of the composition and the balance of cleaning adjunct materials by weight of composition. In other aspects of the present disclosure, the cleaning compositions also comprise amylases at a level of about 0.0001% to about 10%, about 0.001% to about 5%, about 0.001% to about 2%, about 0.005% to about 0.5% amylase by weight of the composition.

In some embodiments of the present disclosure, any suitable pectin degrading enzyme may be used. As used herein, “pectin degrading enzyme(s)” encompass arabinanase (EC 3.2.1.99), galactanases (EC 3.2.1.89), polygalacturonase (EC 3.2.1.15) exo-polygalacturonase (EC 3.2.1.67), exo-poly-alpha-galacturonidase (EC 3.2.1.82), pectin lyase (EC 4.2.2.10), pectin esterase (EC 3.2.1.11), pectate lyase (EC 4.2.2.2), exo-polygalacturonate lyase (EC 4.2.2.9) and hemicellulases such as endo-1,3-β-xylosidase (EC 3.2.1.32), xylan-1,4-β-xylosidase (EC 3.2.1.37) and α-L-arabinofuranosidase (EC 3.2.1.55). Pectin degrading enzymes are natural mixtures of the above mentioned enzymatic activities. Pectin enzymes therefore include the pectin methylesterases which hdyrolyse the pectin methyl ester linkages, polygalacturonases which cleave the glycosidic bonds between galacturonic acid molecules, and the pectin transeliminases or lyases which act on the pectic acids to bring about non-hydrolytic cleavage of α-1,4 glycosidic linkages to form unsaturated derivatives of galacturonic acid.

Suitable pectin degrading enzymes include those of plant, fungal, or microbial origin. In some embodiments, chemically or genetically modified mutants are included. In some embodiments, the pectin degrading enzymes are alkaline pectin degrading enzymes, i.e., enzymes having an enzymatic activity of at least 10%, preferably at least 25%, more preferably at least 40% of their maximum activity at a pH of from about 7.0 to about 12. In certain other embodiments, the pectin degrading enzymes are enzymes having their maximum activity at a pH of from about 7.0 to about 12. Alkaline pectin degrading enzymes are produced by alkalophilic microorganisms e.g., bacterial, fungal, and yeast microorganisms such as Bacillus species. In some embodiments, the microorganisms are Bacillus firmus, Bacillus circulans, and Bacillus subtilis as described in JP 56131376 and JP 56068393. Alkaline pectin decomposing enzymes may include but are not limited to galacturn-1,4-α-galacturonase (EC 3.2.1.67), polygalacturonase activities (EC 3.2.1.15, pectin esterase (EC 3.1.1.11), pectate lyase (EC 4.2.2.2) and their iso enzymes. Alkaline pectin decomposing enzymes can be produced by the Erwinia species. In some embodiments, the alkaline pectin decomposing enzymes are produced by E. chrysanthemi, E. carotovora, E. amylovora, E. herbicola, and E. dissolvens as described in JP 59066588, JP 63042988, and in World J. Microbiol. Microbiotechnol. (8, 2, 115-120) 1992. In certain other embodiments, the alkaline pectin enzymes are produced by Bacillus species as disclosed in JP 73006557 and Agr. Biol. Chem. (1972), 36 (2) 285-93.

In some embodiments of the present disclosure, the disclosed cleaning compositions further comprise pectin degrading enzymes at a level from about 0.00001% to about 10% of additional pectin degrading enzyme by weight of the composition and the balance of cleaning adjunct materials by weight of composition. In other aspects of the present disclosure, the cleaning compositions also comprise pectin degrading enzymes at a level of about 0.0001% to about 10%, about 0.001% to about 5%, about 0.001% to about 2%, about 0.005% to about 0.5% pectin degrading enzyme by weight of the composition.

In some other embodiments, any suitable xyloglucanase finds used in the cleaning compositions of the present disclosure. Suitable xyloglucanases include, but are not limited to those of plant, fungal, or bacterial origin. Chemically or genetically modified mutants are included in some embodiments. As used herein, “xyloglucanase(s)” encompass the family of enzymes described by Vincken and Voragen at Wageningen University [Vincken et al (1994) Plant Physiol., 104, 99-107] and are able to degrade xyloglucans as described in Hayashi et al (1989) Plant. Physiol. Plant Mol. Biol., 40, 139-168. Vincken et al demonstrated the removal of xyloglucan coating from cellulose of the isolated apple cell wall by a xyloglucanase purified from Trichoderma viride (endo-IV-glucanase). This enzyme enhances the enzymatic degradation of cell wall-embedded cellulose and work in synergy with pectic enzymes. Rapidase LIQ+ from Gist-Brocades contains a xyloglucanase activity.

In some embodiments of the present disclosure, the disclosed cleaning compositions further comprise xyloglucanases at a level from about 0.00001% to about 10% of additional xyloglucanase by weight of the composition and the balance of cleaning adjunct materials by weight of composition. In other aspects of the present disclosure, the cleaning compositions also comprise xyloglucanases at a level of about 0.0001% to about 10%, about 0.001% to about 5%, about 0.001% to about 2%, about 0.005% to about 0.5% xyloglucanase by weight of the composition. In certain other embodiments, xyloglucanases for specific applications are alkaline xyloglucanases, i.e., enzymes having an enzymatic activity of at least 10%, preferably at least 25%, more preferably at least 40% of their maximum activity at a pH ranging from 7 to 12. In certain other embodiments, the xyloglucanases are enzymes having their maximum activity at a pH of from about 7.0 to about 12.

In some further embodiments, any suitable cellulase finds used in the cleaning compositions of the present disclosure. Suitable cellulases include, but are not limited to those of bacterial or fungal origin. Chemically or genetically modified mutants are included in some embodiments. Suitable cellulases include, but are not limited to Humicola insolens cellulases (See, e.g., U.S. Pat. No. 4,435,307). Especially suitable cellulases are the cellulases having color care benefits (See, e.g., EP 0 495 257). Commercially available cellulases that find use in the present disclosure include, but are not limited to ENIDOLASE®, CELLUCLEAN®, CELLUZYME®, CAREZYME® (Novozymes A/S, Denmark). Additional commercially available cellulases include PURADEX® (Genencor, A Danisco Division, Palo Alto, Calif.) and KAC-500(B)™ (Kao Corporation). In some embodiments, cellulases are incorporated as portions or fragments of mature wild-type or variant cellulases, wherein a portion of the N-terminus is deleted (See, e.g., U.S. Pat. No. 5,874,276). In some embodiments, the cleaning compositions of the present disclosure further comprise cellulases at a level from about 0.00001% to about 10% of additional cellulase by weight of the composition and the balance of cleaning adjunct materials by weight of composition. In other aspects of the present disclosure, the cleaning compositions also comprise cellulases at a level of about 0.0001% to about 10%, about 0.001% to about 5%, about 0.001% to about 2%, about 0.005% to about 0.5% cellulase by weight of the composition.

In still further embodiments, any lipase suitable for use in detergent compositions also finds use in the present disclosure. Suitable lipases include, but are not limited to those of bacterial or fungal origin. Chemically or genetically modified mutants are included in some embodiments. Examples of useful lipases include Humicola lanuginosa lipase (See, e.g., EP 258 068, and EP 305 216), Rhizomucor miehei lipase (See, e.g., EP 238 023), Candida lipase, such as C. antarctica lipase (e.g., the C. antarctica lipase A or B; see, e.g., EP 214 761), Pseudomonas lipases such as P. alcaligenes lipase and P. pseudoalcaligenes lipase (See, e.g., EP 218 272), P. cepacia lipase (See, e.g., EP 331 376), P. stutzeri lipase (See, e.g., GB 1,372,034), P. fluorescens lipase, Bacillus lipase (e.g., B. subtilis lipase [Dartois et al., (1993) Biochem. Biophys. Acta 1131:253-260]; B. stearothermophilus lipase [See, e.g., JP 64/744992]; and B. pumilus lipase [See, e.g., WO 91/16422]). Furthermore, a number of cloned lipases find use in some embodiments of the present disclosure, including but not limited to Penicillium camembertii lipase (See, Yamaguchi et al., [1991] Gene 103:61-67), Geotricum candidum lipase (See, Schimada et al., [1989] J. Biochem. 106:383-388), and various Rhizopus lipases such as R. delemar lipase (See, Hass et al., [1991] Gene 109:117-113), R. niveus lipase (Kugimiya et al., [1992] Biosci. Biotech. Biochem. 56:716-719), and R. oryzae lipase. Other types of lipolytic enzymes such as cutinases also find use in some embodiments of the present disclosure, including but not limited to the cutinase derived from Pseudomonas mendocina (See, WO 88/09367), and the cutinase derived from Fusarium solani pisi (See, WO 90/09446). Additional suitable lipases include commercially available lipases such as M1 LIPASE™, LUMA FAST™, and LIPOMAX™ (Genencor, A Danisco Division, Palo Alto, Calif.); LIPEX®, LIPOCLEAN®, LIPOLASE® and LIPOLASE® ULTRA (Novozymes A/S, Denmark); and LIPASE P™ “Amano” (Amano Pharmaceutical Co. Ltd., Japan).

In some embodiments, the disclosed cleaning compositions further comprise lipases at a level from about 0.00001% to about 10% of additional lipase by weight of the composition and the balance of cleaning adjunct materials by weight of composition. In other aspects of the present disclosure, the cleaning compositions also comprise lipases at a level of about 0.0001% to about 10%, about 0.001% to about 5%, about 0.001% to about 2%, about 0.005% to about 0.5% lipase by weight of the composition.

In some embodiments, peroxidases are used in combination with hydrogen peroxide or a source thereof (e.g., a percarbonate, perborate or persulfate) in the compositions of the present disclosure. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See, e.g., WO 94/12621 and WO 95/01426). Suitable peroxidases/oxidases include, but are not limited to those of plant, bacterial or fungal origin. Chemically or genetically modified mutants are included in some embodiments. In some embodiments, the cleaning compositions of the present disclosure further comprise peroxidase and/or oxidase enzymes at a level from about 0.00001% to about 10% of additional peroxidase and/or oxidase by weight of the composition and the balance of cleaning adjunct materials by weight of composition. In other aspects of the present disclosure, the cleaning compositions also comprise peroxidase and/or oxidase enzymes at a level of about 0.0001% to about 10%, about 0.001% to about 5%, about 0.001% to about 2%, about 0.005% to about 0.5% peroxidase and/or oxidase enzymes by weight of the composition.

In some embodiments, additional enzymes find use, including but not limited to perhydrolases (See, e.g., WO 05/056782). In addition, in some particularly preferred embodiments, mixtures of the above mentioned enzymes are encompassed herein, in particular one or more additional protease, amylase, lipase, mannanase, and/or at least one cellulase. Indeed, it is contemplated that various mixtures of these enzymes will find use in the present disclosure. It is also contemplated that the varying levels of a polypeptide of the present invention(s) and one or more additional enzymes may both independently range to about 10%, the balance of the cleaning composition being cleaning adjunct materials. The specific selection of cleaning adjunct materials are readily made by considering the surface, item, or fabric to be cleaned, and the desired form of the composition for the cleaning conditions during use (e.g., through the wash detergent use).

Examples of suitable cleaning adjunct materials include, but are not limited to, surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracis, polymeric dispersing agents, clay soil removal agents, structure elasticizing agents, dispersants, suds suppressors, dyes, perfumes, colorants, filler salts, hydrotropes, photoactivators, fluorescers, fabric conditioners, fabric softeners, carriers, hydrotropes, processing aids, solvents, pigments, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See, e.g., U.S. Pat. Nos. 6,610,642; 6,605,458; 5,705,464; 5,710,115; 5,698,504; 5,695,679; 5,686,014; and 5,646,101; all of which are incorporated herein by reference). Embodiments of specific cleaning composition materials are exemplified in detail below. In embodiments in which the cleaning adjunct materials are not compatible with the disclosed a polypeptide of the present inventions in the cleaning compositions, then suitable methods of keeping the cleaning adjunct materials and the endo-β-mannanase(s) separated (i.e., not in contact with each other) until combination of the two components is appropriate are used. Such separation methods include any suitable method known in the art (e.g., gelcaps, encapsulation, tablets, physical separation, etc.).

In some preferred embodiments, an effective amount of one or more polypeptide of the present invention(s) provided herein are included in compositions useful for cleaning a variety of surfaces in need of stain removal. Such cleaning compositions include cleaning compositions for such applications as cleaning hard surfaces, fabrics, and dishes. Indeed, in some embodiments, the present disclosure provides fabric cleaning compositions, while in other embodiments, the present disclosure provides non-fabric cleaning compositions. Notably, the present disclosure also provides cleaning compositions suitable for personal care, including oral care (including dentrifices, toothpastes, mouthwashes, etc., as well as denture cleaning compositions), skin, and hair cleaning compositions. Additionally, in still other embodiments, the present disclosure provides fabric softening compositions. It is intended that the present disclosure encompass detergent compositions in any form (i.e., liquid, granular, bar, solid, semi-solid, gel, paste, emulsion, tablet, capsule, unit dose, sheet, foam etc.).

By way of example, several cleaning compositions wherein the disclosed a polypeptide of the present inventions find use are described in greater detail below. In some embodiments in which the disclosed cleaning compositions are formulated as compositions suitable for use in laundry machine washing method(s), the compositions of the present disclosure preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials). The compositions of the present disclosure also find use detergent additive products in solid or liquid form. Such additive products are intended to supplement and/or boost the performance of conventional detergent compositions and can be added at any stage of the cleaning process. In some embodiments, the density of the laundry detergent compositions herein ranges from about 400 to about 1200 g/liter, while in other embodiments, it ranges from about 500 to about 950 g/liter of composition measured at 20° C.

In embodiments formulated as compositions for use in manual dishwashing methods, the compositions of the disclosure preferably contain at least one surfactant and preferably at least one additional cleaning adjunct material selected from organic polymeric compounds, suds enhancing agents, group II metal ions, solvents, hydrotropes, and additional enzymes.

In some embodiments, various cleaning compositions such as those provided in U.S. Pat. No. 6,605,458 find use with a polypeptide of the present invention. Thus, in some embodiments, the compositions comprising at least one polypeptide of the present invention is a compact granular fabric cleaning composition, while in other embodiments, the composition is a granular fabric cleaning composition useful in the laundering of colored fabrics, in further embodiments, the composition is a granular fabric cleaning composition which provides softening through the wash capacity, in additional embodiments, the composition is a heavy duty liquid fabric cleaning composition. In some embodiments, the compositions comprising at least one polypeptide of the present invention of the present disclosure are fabric cleaning compositions such as those described in U.S. Pat. Nos. 6,610,642 and 6,376,450. In addition, a polypeptide of the present invention find use in granular laundry detergent compositions of particular utility under European or Japanese washing conditions (See, e.g., U.S. Pat. No. 6,610,642).

In some alternative embodiments, the present disclosure provides hard surface cleaning compositions comprising at least one polypeptide of the present invention. Thus, in some embodiments, the compositions comprising at least one polypeptide of the present invention is a hard surface cleaning composition such as those described in U.S. Pat. Nos. 6,610,642; 6,376,450; and 6,376,450.

In yet further embodiments, the present disclosure provides dishwashing compositions comprising at least one polypeptide of the present invention. Thus, in some embodiments, the composition comprising at least one polypeptide of the present invention is a hard surface cleaning composition such as those in U.S. Pat. Nos. 6,610,642 and 6,376,450. In some still further embodiments, the present disclosure provides dishwashing compositions comprising at least one polypeptide of the present invention provided herein. In some further embodiments, the compositions comprising at least one polypeptide of the present invention comprise oral care compositions such as those in U.S. Pat. Nos. 6,376,450 and 6,605,458. The formulations and descriptions of the compounds and cleaning adjunct materials contained in the aforementioned U.S. Pat. Nos. 6,376,450; 6,605,458; and 6,610,642 find use with a polypeptide of the present invention.

In still further embodiments, the compositions comprising at least one polypeptide of the present invention comprise fabric softening compositions such as those in GB-A1 400898, GB-A1 514 276, EP 0 011 340, EP 0 026 528, EP 0 242 919, EP 0 299 575, EP 0 313 146, and U.S. Pat. No. 5,019,292. The formulations and descriptions of the compounds and softening agents contained in the aforementioned GB-A1 400898, GB-A1 514 276, EP 0 011 340, EP 0 026 528, EP 0 242 919, EP 0 299 575, EP 0 313 146, and U.S. Pat. No. 5,019,292 find use with a polypeptide of the present.

The cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,565,422; 5,516,448; 5,489,392; and 5,486,303; all of which are incorporated herein by reference. When a low pH cleaning composition is desired, the pH of such composition is adjusted via the addition of a material such as monoethanolamine or an acidic material such as HCl.

In some embodiments, the cleaning compositions of the present invention are provided in unit dose form, including tablets, capsules, sachets, pouches, sheets, and multi-compartment pouches. In some embodiments, the unit dose format is designed to provide controlled release of the ingredients within a multi-compartment pouch (or other unit dose format). Suitable unit dose and controlled release formats are known in the art (See e.g., EP 2 100 949, WO 02/102955, U.S. Pat. Nos. 4,765,916 and 4,972,017, and WO 04/111178 for materials suitable for use in unit dose and controlled release formats). In some embodiments, the unit dose form is provided by tablets wrapped with a water-soluble film or water-soluble pouches. Various unit dose formats are provided in EP 2 100 947 and WO2013/165725 (which is hereby incorporated by reference), and are known in the art.

While not essential for the purposes of the present disclosure, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to a polypeptide of the present. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282; 6,306,812; and 6,326,348 are incorporated by reference. The aforementioned adjunct ingredients may constitute the balance of the cleaning compositions of the present disclosure.

In some embodiments, the cleaning compositions according to the present disclosure comprise at least one surfactant and/or a surfactant system wherein the surfactant is selected from nonionic surfactants, anionic surfactants, cationic surfactants, ampholytic surfactants, zwitterionic surfactants, semi-polar nonionic surfactants, and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions' acidic contents. In some embodiments, the surfactant is present at a level of from about 0.1% to about 60%, while in alternative embodiments the level is from about 1% to about 50%, while in still further embodiments the level is from about 5% to about 40%, by weight of the cleaning composition.

In some embodiments, the cleaning compositions of the present disclosure contain at least one chelating agent. Suitable chelating agents may include, but are not limited to copper, iron, and/or manganese chelating agents, and mixtures thereof. In embodiments in which at least one chelating agent is used, the cleaning compositions of the present disclosure comprise from about 0.1% to about 15% or even from about 3.0% to about 10% chelating agent by weight of the subject cleaning composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present disclosure. In some preferred embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some preferred embodiments, the anti-redeposition agent is a non-ionic surfactant as known in the art (See, e.g., EP 2 100 949).

In some embodiments, the cleaning compositions of the present disclosure include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In embodiments in which at least one dye transfer inhibiting agent is used, the cleaning compositions of the present disclosure comprise from about 0.0001% to about 10%, from about 0.01% to about 5%, or even from about 0.1% to about 3% by weight of the cleaning composition.

In some embodiments, silicates are included within the compositions of the present disclosure. In some such embodiments, sodium silicates (e.g., sodium disilicate, sodium metasilicate, and crystalline phyllosilicates) find use. In some embodiments, silicates are present at a level of from about 1% to about 20%. In some preferred embodiments, silicates are present at a level of from about 5% to about 15% by weight of the composition.

In some still additional embodiments, the cleaning compositions of the present disclosure also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present disclosure. For example, in some embodiments, the enzymes employed herein are stabilized by the presence of water-soluble sources of zinc (II), calcium (II), and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV). Chlorides and sulfates also find use in some embodiments of the present disclosure. Examples of suitable oligosaccharides and polysaccharides (e.g., dextrins) are known in the art (See, e.g., WO 07/145964). In some embodiments, reversible protease inhibitors also find use, such as boron-containing compounds (e.g., borate, 4-formyl phenyl boronic acid) and/or a tripeptide aldehyde find use to further improve stability, as desired.

In some embodiments, bleaches, bleach activators, and/or bleach catalysts are present in the compositions of the present disclosure. In some embodiments, the cleaning compositions of the present disclosure comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present disclosure (See, e.g., EP 2 100 949).

In some embodiments, bleach activators are used in the compositions of the present disclosure. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Additional bleach activators are known in the art and find use in the present disclosure (See, e.g., EP 2 100 949).

In addition, in some embodiments and as further described herein, the cleaning compositions of the present disclosure further comprise at least one bleach catalyst. In some embodiments, the manganese triazacyclononane and related complexes find use, as well as cobalt, copper, manganese, and iron complexes. Additional bleach catalysts find use in the present disclosure (See, e.g., U.S. Pat. Nos. 4,246,612; 5,227,084; 4,810,410; WO 99/06521; and EP 2 100 949).

In some embodiments, the cleaning compositions of the present disclosure contain one or more catalytic metal complexes. In some embodiments, a metal-containing bleach catalyst finds use. In some preferred embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See, e.g., U.S. Pat. No. 4,430,243). In some embodiments, the cleaning compositions of the present disclosure are catalyzed by means of a manganese compound. Such compounds and levels of use are well known in the art (See, e.g., U.S. Pat. No. 5,576,282). In additional embodiments, cobalt bleach catalysts find use in the cleaning compositions of the present disclosure. Various cobalt bleach catalysts are known in the art (See, e.g., U.S. Pat. Nos. 5,597,936 and 5,595,967) and are readily prepared by known procedures.

In some additional embodiments, the cleaning compositions of the present disclosure include a transition metal complex of a macropolycyclic rigid ligand (MRL). As a practical matter, and not by way of limitation, in some embodiments, the compositions and cleaning processes provided by the present disclosure are adjusted to provide on the order of at least one part per hundred million of the active MRL species in the aqueous washing medium, and in some preferred embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, preferred transition-metals in the instant transition-metal bleach catalyst include, but are not limited to manganese, iron, and chromium. Preferred MRLs also include, but are not limited to special ultra-rigid ligands that are cross-bridged (e.g., 5,12-diethyl-1,5,8,12-tetraazabicyclo[6.6.2] hexadecane). Suitable transition metal MRLs are readily prepared by known procedures (See, e.g., WO 2000/32601 and U.S. Pat. No. 6,225,464).

In some embodiments, the cleaning compositions of the present disclosure comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2 100 949, WO 94/26860, and WO 94/26859). In some embodiments, the metal care agent is a zinc salt. In some further embodiments, the cleaning compositions of the present disclosure comprise from about 0.1% to about 5% by weight of one or more metal care agent.

As indicated above, the cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,516,448; 5,489,392; and 5,486,303; all of which are incorporated herein by reference. In some embodiments in which a low pH cleaning composition is desired, the pH of such composition is adjusted via the addition of an acidic material such as HCl.

The cleaning compositions disclosed herein of find use in cleaning a situs (e.g., a surface, dishware, or fabric). Typically, at least a portion of the situs is contacted with an embodiment of the present cleaning composition, in neat form or diluted in wash liquor, and then the situs is optionally washed and/or rinsed. For purposes of the present disclosure, “washing” includes but is not limited to, scrubbing and mechanical agitation. In some embodiments, the cleaning compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 30:1.

### Polypeptides of the Present Invention as Chemical Reagents

The preference of a polypeptide of the present invention for hydrolysis of polysaccharide chains containing mannose units, including, but not limited to, mannans, galactomannans, and glucomannans, makes the present polypeptides particularly useful for performing mannan hydrolysis reactions involving polysaccharide substrates containing 1,4β-D-mannosidic linkages.

In general terms, a donor molecule is incubated in the presence of an isolated polypeptide or a polypeptide described herein or fragment or variant thereof under conditions suitable for performing a mannan hydrolysis reaction, followed by, optionally, isolating a product from the reaction. Alternatively, in the context of a foodstuff, the product may become a component of the foodstuff without isolation. In certain embodiments, the donor molecule is a polysaccharide chain comprising mannose units, including but not limited to mannans, glucomannans, galactomannans, and galactoglucomannans.

Polypeptides of the Present Invention for Food Processing and/or Animal Feed

In one embodiment, a composition comprising a polypeptide described herein is used to process and/or manufacture animal feed or food for humans. In yet a further embodiment, a polypeptide of the present invention can be an additive to feed for non-human animals. In another embodiment, a polypeptide of the present invention can be useful for human food, such as, for example, as an additive to human food.

Several nutritional factors can limit the amount of inexpensive plant material that can be used to prepare animal feed and food for humans. For example, plant material containing oligomannans such as mannan, galactomannan, glucomannan and galactoglucomannan can reduce an animal's ability to digest and absorb nutritional compounds such as minerals, vitamins, sugars, and fats. These negative effects are in particular due to the high viscosity of the mannan-containing polymers and to the ability of the mannan-containing polymers to absorb nutritional compounds. These effects can be reduced by including an enzyme in the feed that degrades the mannan-containing polymers, such as, an endo-β-mannanase enzyme described herein, thereby enabling a higher proportion of mannan-containing polymers typically found in inexpensive plant material to be included in the feed, which ultimately reduces the cost of the feed. Additionally, a polypeptide described herein can breakdown the mannan-containing polymers into simpler sugars, which can be more readily assimilated to provide additional energy.

In a further embodiment, animal feed containing plant material is incubated in the presence of a polypeptide and/or isolated polypeptide described herein or fragment or variant thereof under conditions suitable for breaking down mannan-containing polymers.

In another embodiment, a bread improver composition comprises a polypeptide described herein, optionally in combination with a source of mannan or glucomannan or galactomannan, and further optionally in combination with one or more other enzymes.

The term non-human animal includes all non-ruminant and ruminant animals. In a particular embodiment, the non-ruminant animal is selected from the group consisting of, but is not limited to, horses and monogastric animals such as, but not limited to, pigs, poultry, swine and fish. In further embodiments, the pig may be, but is not limited to, a piglet, a growing pig, and a sow; the poultry may be, but is not limited to, a turkey, a duck and a chicken including, but not limited to, a broiler chick and a layer; and fish including but not limited to salmon, trout, tilapia, catfish and carps; and crustaceans including but not limited to shrimps and prawns. In a further embodiment, the ruminant animal is selected from the group consisting of, but is not limited to, cattle, young calves, goats, sheep, giraffes, bison, moose, elk, yaks, water buffalo, deer, camels, alpacas, llamas, antelope, pronghorn, and nilgai.

In some embodiments, a polypeptide of the present invention is used to pretreat feed instead of as a feed additive. In some preferred embodiment, a polypeptide of the present invention is added to, or used to pretreat, feed for weanling pigs, nursery pigs, piglets, fattening pigs, growing pigs, finishing pigs, laying hens, broiler chicks, and turkeys.

In another embodiment, a polypeptide of the present invention is added to, or used to pretreat, feed from plant material such as palm kernel, coconut, konjac, locust bean gum, gum guar, soy beans, barley, oats, flax, wheat, corn, linseed, citrus pulp, cottonseed, groundnut, rapeseed, sunflower, peas, and lupines.

A polypeptide in accordance with the present invention isthermostable, and as a result, a polypeptide disclosed herein can be used in processes of producing pelleted feed in which heat is applied to the feed mixture before the pelleting step. In another embodiment, a polypeptide of the present invention is added to the other feed ingredients either in advance of the pelleting step or after the pelleting step (i.e to the already formed feed pellets).

In yet another embodiment, food processing or feed supplement compositions that containa polypeptide described herein may optionally further contain other substituents selected from coloring agents, aroma compounds, stabilizers, vitamins, minerals, and other feed or food enhancing enzymes. This applies in particular to the so-called pre-mixes.

In a still further embodiment, a food additive according to the present invention may be combined in an appropriate amount with other food components, such as, for example, a cereal or plant protein to form a processed food product.

In one embodiment, an animal feed composition and/or animal feed additive composition and/or pet food comprises a polypeptide described herein.

Another embodiment relates to a method for preparing an animal feed composition and/or animal feed additive composition and/or pet food comprising mixing a polypeptide described herein with one or more animal feed ingredients and/or animal feed additive ingredients and/or pet food ingredients.

A further embodiment relates to the use of a polypeptide described herein to prepare an animal feed composition and/or animal feed additive composition and/or pet food. The phrase “pet food” means food for a household animal such as, but not limited to, dogs; cats; gerbils; hamsters; chinchillas; fancy rats; guinea pigs; avian pets, such as canaries, parakeets, and parrots; reptile pets, such as turtles, lizards and snakes; and aquatic pets, such as tropical fish and frogs.

The terms animal feed composition, feedstuff and fodder are used interchangeably and may comprise one or more feed materials selected from the group comprising a) cereals, such as small grains (e.g., wheat, barley, rye, oats and combinations thereof) and/or large grains such as maize or sorghum; b) by-products from cereals, such as corn gluten meal, Distillers Dried Grain Solubles (DDGS) (particularly corn based Distillers Dried Grain Solubles (cDDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, and sesame; d) oils and fats obtained from vegetable and animal sources; and e) minerals and vitamins.

In one aspect, the food composition or additive may be liquid or solid.

### Polypeptides of the Present Invention for Fermented Beverages, Such as Beer

In an aspect of the invention the food composition is a beverage, including, but not limited to, a fermented beverage such as beer and wine, comprising a polypeptide described herein.

In the context of the present invention, the term “fermented beverage” is meant to comprise any beverage produced by a method comprising a fermentation process, such as a microbial fermentation, such as a bacterial and/or yeast fermentation.

In an aspect of the invention the fermented beverage is beer. The term “beer” is meant to comprise any fermented wort produced by fermentation/brewing of a starch-containing plant material. Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. Examples of adjuncts include, for example, common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like may be used as a source of starch

As used herein, the term “mash” refers to an aqueous slurry of any starch and/or sugar containing plant material such as grist, e. g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

In another aspect the invention relates to a method of preparing a fermented beverage such as beer comprising mixing any polypeptide of the present invention with a malt and/or adjunct.

Examples of beers comprise: full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, as well as alternative cereal and malt beverages such as fruit flavoured malt beverages, e. g. citrus flavoured, such as lemon-, orange-, lime-, or berry-flavoured malt beverages; liquor flavoured malt beverages, e. g., vodka-, rum-, or tequila-flavoured malt liquor; or coffee flavoured malt beverages, such as caffeine-flavoured malt liquor; and the like.

One aspect of the invention relates to the use of any polypeptide of the present invention in the production of a fermented beverage, such as a beer.

Another aspect concerns a method of providing a fermented beverage comprising the step of contacting a mash and/or a wort with any polypeptide of the present invention.

A further aspect relates to a method of providing a fermented beverage comprising the steps of: (a) preparing a mash, (b) filtering the mash to obtain a wort, and (c) fermenting the wort to obtain a fermented beverage, such as a beer, wherein any polypeptide of the present invention is added to: (i) the mash of step (a) and/or (ii) the wort of step (b) and/or (iii) the wort of step (c).

According to yet another aspect, a fermented beverage, such as a beer, is produced or provided by a method comprising the step(s) of (1) contacting a mash and/or a wort with any polypeptide of the present invention; and/or (2) (a) preparing a mash, (b) filtering the mash to obtain a wort, and (c) fermenting the wort to obtain a fermented beverage, such as a beer, wherein any polypeptide of the present invention is added to: (i) the mash of step (a) and/or (ii) the wort of step (b) and/or (iii) the wort of step (c).

### Polypeptides of the Present Invention for Treating Coffee Extracts

A polypeptide of the present inventions described herein may also be used for hydrolyzing galactomannans present in liquid coffee extracts. In one aspect, a polypeptide of the present invention is used to inhibit gel formation during freeze drying of liquid coffee extracts. The decreased viscosity of the extract reduces the energy consumption during drying. In certain other aspects, a polypeptide of the present inventions is applied in an immobilized form in order to reduce enzyme consumption and avoid contamination of the coffee extract. This use is further disclosed in EP 676 145.

In general terms the coffee extract is incubated in the presence of a polypeptide and/or isolated polypepetide of the present invention or fragment or variant thereof under conditions suitable for hydrolyzing galactomannans present in liquid coffee extract.

### Polypeptides of the Present Invention for Use in Bakery Food Products

In another aspect the invention relates to a method of preparing baked products comprising addition of any polypeptide of the invention to dough, followed by baking the dough. Examples of baked products are well known to those skilled in the art and include breads, rolls, puff pastries, sweet fermented doughs, buns, cakes, crackers, cookies, biscuits, waffles, wafers, tortillas, breakfast cereals, extruded products, and the like.

Any polypeptide of the invention may be added to dough as part of a bread improver composition. Bread improvers are compositions containing a variety of ingredients, which improve dough properties and the quality of bakery products, e.g. bread and cakes. Bread improvers are often added in industrial bakery processes because of their beneficial effects e.g. the dough stability and the bread texture and volume. Bread improvers usually contain fats and oils as well as additives like emulsifiers, enzymes, antioxidants, oxidants, stabilizers and reducing agents. In addition to any of the polypeptides of the present invention, other enzymes which may also be present in the bread improver or which may be otherwise used in conjunction with any of the polypeptides of the present invention include amylases, hemicellulases, amylolytic complexes, lipases, proteases, xylanases, pectinases, pullulanases, non starch polysaccharide degrading enzymes and redox enzymes like glucose oxidase, lipoxygenase or ascorbic acid oxidase.

In a preferred bakery aspect of the current invention, any of the polypeptides of the invention may be added to dough as part of a bread improver composition which also comprises a glucomannan and/or galactomannan source such as konjac gum, guar gum, locust bean gum (Ceratonia siliqua), copra meal, ivory nut mannan (Phyteleohas macrocarpa), seaweed mannan extract, coconut meal, and the cell wall of brewers yeast (may be dried, or used in the form of brewers yeast extract). Other acceptable mannan derivatives for use in the current invention include unbranched β-1,4-linked mannan homopolymer and manno-oligosaccharides (mannobiose, mannotriose, mannotetraose and mannopentoase). Any polypeptide of the invention can be further used either alone, or in combination with a glucomannan and/or galactomannan and/or galactoglucomannan to improve the dough tolerance; dough flexibility and/or dough stickiness; and/or bread crumb structure, as well as retarding staling of the bread. In another aspect, the mannanase hydrolysates act as soluble prebiotics such as manno-oligosaccharides (MOS) which promote the growth of lactic acid bacteria commonly associated with good health when found at favourable population densities in the colon.

In one aspect, the dough to which any polypeptide of the invention is added comprises bran or oat, rice, millet, maize, or legume flour in addition to or instead of pure wheat flour (i.e., is not a pure white flour dough).

### Polypeptides of the Present Invention for Use in Dairy Food Products

In one aspect of the invention, any polypeptide of the invention may be added to milk or any other dairy product to which has also been added a glucomannan and/or galactomannan. Typical glucomannan and/or galactomannan sources are listed above in the bakery aspects, and include guar or konjac gum. The combination of any polypeptide of the invention with a glucomannan and/or galactomannan releases mannanase hydrolysates (mannooligosaccharides) which act as soluble prebiotics by promoting the selective growth and proliferation of probiotic bacteria (especially Bifidobacteria and Lactobacillus lactic acid bacteria) commonly associated with good health when found at favourable population densities in the large intestine or colon.

Another aspectrelates to a method of preparing milk or dairy products comprising addition of any polypeptide of the invention and any glucomannan or galactomannan or galactoglucomannan.

In another aspect, any polypeptide of the invention is used in combination with any glucomannan or galactomannan prior to or following addition to a dairy based foodstuff to produce a dairy based foodstuff comprising prebiotic mannan hydrolysates. In a further aspect, the thusly produced mannooligosacharide-containing dairy product is capable of increasing the population of beneficial human intestinal microflora, and in a yet further aspect the dairy based foodstuff may comprise any polypeptide of the invention together with any source of glucomannan and/or galactomannan and/or galactoglucomannan, and a dose sufficient for inoculation of at least one strain of bacteria (such as Bifidobacteria or Lactobacillus) known to be of benefit in the human large intestine. In one aspect, the dairy-based foodstuff is a yoghurt or milk drink.

### Polypeptides of the Present Invention for Paper Pulp Bleaching

The polypeptides described herein find further use in the enzyme aided bleaching of paper pulps such as chemical pulps, semi-chemical pulps, kraft pulps, mechanical pulps, and pulps prepared by the sulfite method. In general terms, paper pulps are incubated with a polypeptide and/or isolated polypeptide or fragment or variant thereof described herein under conditions suitable for bleaching the paper pulp.

In some embodiments, the pulps are chlorine free pulps bleached with oxygen, ozone, peroxide or peroxyacids. In some embodiments, a polypeptide of the invention is used in enzyme aided bleaching of pulps produced by modified or continuous pulping methods that exhibit low lignin contents. In some other embodiments, a polypeptide of the present invention is applied alone or preferably in combination with xylanase and/or endoglucanase and/or alpha-galactosidase and/or cellobiohydrolase enzymes.

### Polypeptides of the Present Invention for Degrading Thickeners

Galactomannans such as guar gum and locust bean gum are widely used as thickening agents e.g., in food and print paste for textile printing such as prints on T-shirts. Thus, a polypeptide described herein also finds use in reducing the thickness or viscosity of mannan-containing substrates. In certain embodiments, a polypeptide described herein is used for reducing the viscosity of residual food in processing equipment thereby facilitating cleaning after processing. In certain other embodiments, a polypeptide disclosed herein is used for reducing viscosity of print paste, thereby facilitating wash out of surplus print paste after textile printings. In general terms, a mannan-containing substrate is incubated with a polypeptide and/or isolated polypeptide or fragment or variant thereof described herein under conditions suitable for reducing the viscosity of the mannan-containing substrate.

Other aspects and embodiments of the present compositions and methods will be apparent from the foregoing description and following examples.

### EXAMPLES

The following examples are provided to demonstrate and illustrate certain preferred embodiments and aspects of the present disclosure and should not be construed as limiting.

### Example 1

### Identification of Bacillus and Paenibacillus Mannanases

The following nucleotide and amino acid sequences for mannanases encoded by Bacillus and Paenibacillus species were extracted from the NCBI Database.

The nucleotide sequence of the BciMan1 gene (NCBI Reference Sequence AB007123.1) isolated from B. circulans K−1 is set forth as SEQ ID NO:1 (the sequence encoding the predicted native signal peptide is shown in bold):

ATGGGGTGGTTTTTAGTGATTTTACGCAAGTGGTTGATTGCTTTTGTCGC
ATTTTTACTGATGTTCTCGTGGACTGGACAACTTACGAACAAAGCACATG
CTGCAAGCGGATTTTATGTAAGCGGTACCAAATTATTGGATGCTACAGGA
CAACCATTTGTGATGCGAGGAGTCAATCATGCGCACACATGGTATAAAGA
TCAACTATCCACCGCAATACCAGCCATTGCTAAAACAGGTGCCAACACGA
TACGTATTGTACTGGCGAATGGACACAAATGGACGCTTGATGATGTAAAC
ACCGTCAACAATATTCTCACCCTCTGTGAACAAAACAAACTAATTGCCGT
TTTGGAAGTACATGACGCTACAGGAAGCGATAGTCTTTCCGATTTAGACA
ACGCCGTTAATTACTGGATTGGTATTAAAAGCGCGTTGATCGGCAAGGAA
GACCGTGTAATCATTAATATAGCTAACGAGTGGTACGGAACATGGGATGG
AGTCGCCTGGGCTAATGGTTATAAGCAAGCCATACCCAAACTGCGTAATG
CTGGTCTAACTCATACGCTGATTGTTGACTCCGCTGGATGGGGACAATAT
CCAGATTCGGTCAAAAATTATGGGACAGAAGTACTGAATGCAGACCCGTT
AAAAAACACAGTATTCTCTATCCATATGTATGAATATGCTGGGGGCAATG
CAAGTACCGTCAAATCCAATATTGACGGTGTGCTGAACAAGAATCTTGCA
CTGATTATCGGCGAATTTGGTGGACAACATACAAACGGTGATGTGGATGA
AGCCACCATTATGAGTTATTCCCAAGAGAAGGGAGTCGGCTGGTTGGCTT
GGTCCTGGAAGGGAAATAGCAGTGATTTGGCTTATCTCGATATGACAAAT
GATTGGGCTGGTAACTCCCTCACCTCGTTCGGTAATACCGTAGTGAATGG
CAGTAACGGCATTAAAGCAACTTCTGTGTTATCCGGCATTTTTGGAGGTG
TTACGCCAACCTCAAGCCCTACTTCTACACCTACATCTACGCCAACCTCA
ACTCCTACTCCTACGCCAAGTCCGACCCCGAGTCCAGGTAATAACGGGAC
GATCTTATATGATTTCGAAACAGGAACTCAAGGCTGGTCGGGAAACAATA
TTTCGGGAGGCCCATGGGTCACCAATGAATGGAAAGCAACGGGAGCGCAA
ACTCTCAAAGCCGATGTCTCCTTACAATCCAATTCCACGCATAGTCTATA
TATAACCTCTAATCAAAATCTGTCTGGAAAAAGCAGTCTGAAAGCAACGG
TTAAGCATGCGAACTGGGGCAATATCGGCAACGGGATTTATGCAAAACTA
TACGTAAAGACCGGGTCCGGGTGGACATGGTACGATTCCGGAGAGAATCT
GATTCAGTCAAACGACGGTACCATTTTGACACTATCCCTCAGCGGCATTT
CGAATTTGTCCTCAGTCAAAGAAATTGGGGTAGAATTCCGCGCCTCCTCA
AACAGTAGTGGCCAATCAGCTATTTATGTAGATAGTGTTAGTCTGCAATG
A.

The amino acid sequence of the precursor protein encoded by the BciMan1 gene, BciMan1 (NCBI Accession No. BAA25878.1) is set forth as SEQ ID NO:2 (the predicted native signal peptide is shown in bold):

MGWFLVILRKWLIAFVAFLLMFSWTGQLTNKAHAASGFYVSGTKLLDATG
QPFVMRGVNHAHTWYKDQLSTAIPAIAKTGANTIRIVLANGHKWTLDDVN
TVNNILTLCEQNKLIAVLEVHDATGSDSLSDLDNAVNYWIGIKSALIGKE
DRVIINIANEWYGTWDGVAWANGYKQAIPKLRNAGLTHTLIVDSAGWGQY
PDSVKNYGTEVLNADPLKNTVFSIHMYEYAGGNASTVKSNIDGVLNKNLA
LIIGEFGGQHTNGDVDEATIMSYSQEKGVGWLAWSWKGNSSDLAYLDMTN
DWAGNSLTSFGNTVVNGSNGIKATSVLSGIFGGVTPTSSPTSTPTSTPTS
TPTPTPSPTPSPGNNGTILYDFETGTQGWSGNNISGGPWVTNEWKATGAQ
TLKADVSLQSNSTHSLYITSNQNLSGKSSLKATVKHANWGNIGNGIYAKL
YVKTGSGWTWYDSGENLIQSNDGTILTLSLSGISNLSSVKEIGVEFRASS
NSSGQSAIYVDSVSLQ.

The nucleic acid sequence for the BciMan3 gene (NCBI Reference Sequence AY907668.1, from 430 to 1413, complement) isolated from B. circulans 196 is set forth as SEQ ID NO:3 (the sequence encoding the predicted native signal peptide is shown in bold):

ATGATGTTGATATGGATGCAGGGATGGAAGTCTATTCTAGTCGCGATCTT
GGCGTGTGTGTCAGTAGGCGGTGGGCTTCCTAGTCCAGAAGCAGCCACAG
GATTTTATGTAAACGGTACCAAGCTGTATGATTCAACGGGCAAGGCCTTT
GTGATGAGGGGTGTAAATCATCCCCACACCTGGTACAAGAATGATCTGAA
CGCGGCTATTCCGGCTATCGCGCAAACGGGAGCCAATACCGTACGAGTCG
TCTTGTCGAACGGGTCGCAATGGACCAAGGATGACCTGAACTCCGTCAAC
AGTATCATCTCGCTGGTGTCGCAGCATCAAATGATAGCCGTTCTGGAGGT
GCATGATGCGACAGGCAAAGATGAGTATGCTTCCCTTGAAGCGGCCGTCG
ACTATTGGATCAGCATCAAAGGGGCATTGATCGGAAAAGAAGACCGCGTC
ATCGTCAATATTGCTAATGAATGGTATGGAAATTGGAACAGCAGCGGATG
GGCCGATGGTTATAAGCAGGCCATTCCCAAATTAAGAAACGCGGGCATTA
AGAATACGTTGATCGTTGATGCAGCGGGATGGGGGCAATACCCGCAATCC
ATCGTGGATGAGGGGGCCGCGGTATTTGCTTCCGATCAACTGAAGAATAC
GGTATTCTCCATCCATATGTATGAGTATGCCGGTAAGGATGCCGCTACGG
TGAAAACGAATATGGACGATGTTTTAAACAAAGGATTGCCTTTAATCATT
GGGGAGTTCGGCGGCTATCATCAAGGTGCCGATGTCGATGAGATTGCTAT
TATGAAGTACGGACAGCAGAAGGAAGTGGGCTGGCTGGCTTGGTCCTGGT
ACGGAAACAGCCCGGAGCTGAACGATTTGGATCTGGCTGCAGGGCCAAGC
GGAAACCTGACCGGCTGGGGAAACACGGTGGTTCATGGAACCGACGGGAT
TCAGCAAACCTCCAAGAAAGCGGGCATTTATTAA.

The amino acid sequence of the precursor protein encoded by the BciMan3 gene, BciMan3 (NCBI Accession No. AAX87002.1) is set forth as SEQ ID NO:4 (the predicted native signal peptide is shown in bold):

MMLIWMQGWKSILVAILACVSVGGGLPSPEAATGFYVNGTKLYDSTGKAF
VMRGVNHPHTWYKNDLNAAIPAIAQTGANTVRVVLSNGSQWTKDDLNSVN
SIISLVSQHQMIAVLEVHDATGKDEYASLEAAVDYWISIKGALIGKEDRV
IVNIANEWYGNWNSSGWADGYKQAIPKLRNAGIKNTLIVDAAGWGQYPQS
IVDEGAAVFASDQLKNTVFSIHMYEYAGKDAATVKTNMDDVLNKGLPLII
GEFGGYHQGADVDEIAIMKYGQQKEVGWLAWSWYGNSPELNDLDLAAGPS
GNLTGWGNTVVHGTDGIQQTSKKAGIY.

The nucleic acid sequence for the BciMan4 gene (NCBI Reference Sequence AY913796.1, from 785 to 1765) isolated from Bacillus circulans CGMCC1554 is set forth as SEQ ID NO:5 (the sequence encoding the predicted native signal peptide is shown in bold):

ATGGCCAAGTTGCAAAAGGGTACAATCTTAACAGTCATTGCAGCACTGAT
GTTTGTCATTTTGGGGAGCGCGGCGCCCAAAGCCGCAGCAGCTACAGGTT
TTTACGTGAATGGAGGCAAATTGTACGATTCTACGGGTAAACCATTTTAC
ATGAGGGGTATCAATCATGGGCACTCCTGGTTTAAAAATGATTTGAACAC
GGCTATCCCTGCGATCGCAAAAACGGGTGCCAATACGGTACGAATTGTTT
TATCAAACGGTACACAATACACCAAGGATGATCTGAATTCCGTAAAAAAC
ATCATTAATGTCGTAAATGCAAACAAGATGATTGCTGTGCTTGAAGTACA
CGATGCCACTGGGAAAGATGACTTCAACTCGTTGGATGCAGCGGTCAACT
ACTGGATAAGCATCAAAGAAGCACTGATCGGGAAGGAAGATCGGGTTATT
GTAAACATTGCAAACGAGTGGTACGGAACATGGAACGGAAGCGCGTGGGC
TGACGGGTACAAAAAAGCTATTCCGAAATTAAGAGATGCGGGTATTAAAA
ATACCTTGATTGTAGATGCAGCAGGCTGGGGTCAGTACCCTCAATCGATC
GTCGATTACGGACAAAGCGTATTCGCCGCGGATTCACAGAAAAATACGGC
GTTTTCCATTCACATGTATGAGTATGCAGGCAAGGATGCGGCCACCGTCA
AATCCAATATGGAAAATGTGCTGAATAAGGGGCTGGCCTTAATCATTGGT
GAGTTCGGAGGATATCACACCAATGGAGATGTCGATGAATATGCAATCAT
GAAATATGGTCTGGAAAAAGGGGTAGGATGGCTTGCATGGTCTTGGTACG
GTAATAGCTCTGGATTAAACTATCTTGATTTGGCAACAGGACCTAACGGC
AGTTTGACGAGCTATGGTAATACGGTTGTCAATGATACTTACGGAATTAA
AAATACGTCCCAAAAAGCGGGAATCTTTTAA.

The amino acid sequence of the precursor protein encoded by the BciMan4 gene, BciMan4 (NCBI Accession No. AAX87003.1) is set forth as SEQ ID NO:6 (the predicted native signal peptide is shown in bold):

MAKLQKGTILTVIAALMFVILGSAAPKAAAATGFYVNGGKLYDSTGKPFY
MRGINHGHSWFKNDLNTAIPAIAKTGANTVRIVLSNGTQYTKDDLNSVKN
IINVVNANKMIAVLEVHDATGKDDFNSLDAAVNYWISIKEALIGKEDRVI
VNIANEWYGTWNGSAWADGYKKAIPKLRDAGIKNTLIVDAAGWGQYPQSI
VDYGQSVFAADSQKNTAFSIHMYEYAGKDAATVKSNMENVLNKGLALIIG
EFGGYHTNGDVDEYAIMKYGLEKGVGWLAWSWYGNSSGLNYLDLATGPNG
SLTSYGNTVVNDTYGIKNTSQKAGIF.

The nucleic acid sequence for the PpoMan1 gene (NCBI Reference Sequence NC_014483.1, from 649134 to 650117, complement) isolated from Paenibacillus polymyxa E681 is set forth as SEQ ID NO:7 (the sequence encoding the predicted native signal peptide is shown in bold):

ATGAAGGTATTGTTAAGAAAAGCATTATTGTCTGGACTGGTCGGCTTGCT
CATCATGATTGGTTTAGGAGGAGTTTTCTCCAAGGTAGAAGCTGCTTCAG
GATTTTATGTAAGCGGTACCAAATTGTATGACTCTACAGGCAAGCCATTT
GTTATGAGAGGCGTCAATCATGCTCACACTTGGTACAAAAACGATCTTTA
TACAGCTATCCCGGCAATTGCCCAGACAGGTGCTAATACCGTCCGAATTG
TCCTTTCTAACGGAAACCAGTACACCAAGGATGACATTAATTCCGTGAAA
AATATTATCTCTCTTGTCTCCAACTATAAAATGATTGCTGTACTTGAAGT
TCATGATGCTACAGGCAAAGACGACTACGCGTCTTTGGATGCAGCTGTGA
ACTACTGGATTAGCATAAAAGATGCTCTGATCGGCAAGGAAGACCGGGTT
ATCGTAAACATTGCGAACGAATGGTATGGTTCTTGGAATGGAAGTGGTTG
GGCTGATGGATACAAGCAAGCGATTCCCAAGTTGAGAAACGCAGGTATCA
AAAATACGCTCATCGTCGATTGTGCCGGATGGGGACAGTATCCTCAGTCT
ATCAATGACTTTGGTAAATCTGTATTTGCAGCTGATTCTTTGAAGAATAC
GGTATTCTCTATTCATATGTATGAGTTCGCTGGTAAAGATGCTCAAACCG
TTCGAACCAATATTGATAACGTTCTGAATCAAGGAATTCCTCTGATTATT
GGTGAATTTGGAGGTTACCACCAGGGAGCAGACGTCGACGAGACAGAAAT
CATGAGATATGGCCAATCCAAAGGAGTAGGCTGGTTAGCCTGGTCCTGGT
ATGGTAATAGTTCCAACCTTTCCTACCTTGATCTTGTAACAGGACCTAAT
GGCAATCTGACGGATTGGGGAAAAACTGTAGTTAACGGAAGCAACGGGAT
CAAAGAAACATCGAAAAAAGCTGGTATCTACTAA.

The amino acid sequence of the protein encoded by the PpoMan1 gene, PpoMan1 (NCBI Accession No. YP_003868989.1) is set forth as SEQ ID NO:8 (the predicted native signal peptide is shown in bold):

MKVLLRKALLSGLVGLLIMIGLGGVFSKVEAASGFYVSGTKLYDSTGKPF
VMRGVNHAHTWYKNDLYTAIPAIAQTGANTVRIVLSNGNQYTKDDINSVK
NIISLVSNYKMIAVLEVHDATGKDDYASLDAAVNYWISIKDALIGKEDRV
IVNIANEWYGSWNGSGWADGYKQAIPKLRNAGIKNTLIVDCAGWGQYPQS
INDFGKSVFAADSLKNTVFSIHMYEFAGKDAQTVRTNIDNVLNQGIPLII
GEFGGYHQGADVDETEIMRYGQSKGVGWLAWSWYGNSSNLSYLDLVTGPN
GNLTDWGKTVVNGSNGIKETSKKAGIY.

The nucleic acid sequence for the PpoMan2 gene (NCBI Reference Sequence NC_014622.1, from 746871 to 747854, complement) isolated from Paenibacillus polymyxa SC2 is set forth as SEQ ID NO:9 (the sequence encoding the predicted native signal peptide is shown in bold):

GTGAACGCATTGTTAAGAAAAGCATTATTGTCTGGACTCGCTGGTCTGCT
TATCATGATTGGTTTGGGGGGATTCTTCTCCAAGGCGCAAGCTGCTTCAG
GATTTTATGTAAGCGGTACCAATCTGTATGACTCTACAGGCAAACCGTTC
GTTATGAGAGGCGTCAATCATGCTCACACTTGGTACAAAAACGATCTTTA
TACTGCTATCCCAGCAATTGCTAAAACAGGTGCTAATACAGTCCGAATTG
TCCTTTCTAACGGAAACCAGTACACCAAGGATGACATTAATTCCGTGAAA
AATATTATCTCTCTCGTCTCCAACCATAAAATGATTGCTGTACTTGAAGT
TCATGACGCTACAGGTAAAGACGACTATGCGTCTTTGGATGCAGCAGTGA
ATTACTGGATTAGTATAAAAGATGCTCTGATCGGCAAGGAAGATCGGGTT
ATCGTGAACATTGCGAACGAATGGTATGGCTCTTGGAATGGAGGCGGTTG
GGCAGATGGGTATAAGCAAGCGATTCCCAAGCTGAGAAACGCAGGCATCA
AAAATACGCTCATCGTCGATTGTGCTGGATGGGGACAATACCCTCAGTCT
ATCAATGACTTTGGTAAATCTGTGTTTGCAGCTGATTCTTTGAAAAATAC
CGTTTTCTCCATTCATATGTATGAATTTGCTGGCAAAGATGTTCAAACGG
TTCGAACCAATATTGATAACGTTCTGTATCAAGGGCTCCCTTTGATTATT
GGTGAATTTGGCGGTTACCATCAGGGAGCAGACGTCGACGAGACAGAAAT
CATGAGATACGGCCAATCTAAAAGCGTAGGCTGGTTAGCCTGGTCCTGGT
ATGGCAATAGCTCCAACCTTAATTATCTTGATCTTGTGACAGGACCTAAC
GGCAATCTGACCGATTGGGGTCGCACCGTGGTAGAGGGAGCCAACGGGAT
CAAAGAAACATCGAAAAAAGCGGGTATCTTCTAA.

The amino acid sequence of the hypothetical protein encoded by the PpoMan2 gene, PpoMan2 (NCBI Accession No. YP_003944884.1) is set forth as SEQ ID NO:10 (the predicted native signal peptide is shown in bold):

MNALLRKALLSGLAGLLIMIGLGGFFSKAQAASGFYVSGTNLYDSTGKPF
VMRGVNHAHTWYKNDLYTAIPAIAKTGANTVRIVLSNGNQYTKDDINSVK
NIISLVSNHKMIAVLEVHDATGKDDYASLDAAVNYWISIKDALIGKEDRV
IVNIANEWYGSWNGGGWADGYKQAIPKLRNAGIKNTLIVDCAGWGQYPQS
INDFGKSVFAADSLKNTVFSIHMYEFAGKDVQTVRTNIDNVLYQGLPLII
GEFGGYHQGADVDETEIMRYGQSKSVGWLAWSWYGNSSNLNYLDLVTGPN
GNLTDWGRTVVEGANGIKETSKKAGIF.

The nucleic acid sequence for the PspMan4 gene (NCBI Reference Sequence GQ358926.1) isolated from Paenibacillus sp. A1 is set forth as SEQ ID NO:11 (the sequence encoding the predicted native signal peptide is shown in bold):

ATGAAATACCTGCTGCCGACCGCTGCTGCTGGTCTGCTGCTCCTCGCTGC
CCAGCCGGCGATGGCCATGGCTACAGGTTTTTATGTAAGCGGTAACAAGT
TATACGATTCCACTGGCAAGCCTTTTGTTATGAGAGGTGTTAATCACGGA
CATTCCTGGTTCAAAAATGATTTGAATACCGCTATCCCTGCCATCGCCAA
AACAGGTGCCAATACGGTACGCATTGTTCTTTCGAATGGTAGCCTGTACA
CCAAAGATGATCTGAACGCTGTTAAAAATATTATTAATGTGGTTAACCAG
AATAAAATGATAGCTGTACTCGAAGTACATGACGCCACAGGGAAAGATGA
CTATAATTCGTTGGATGCGGCGGTGAACTACTGGATTAGTATTAAGGAAG
CTTTGATTGGAAAAGAAGATCGGGTAATTGTCAACATCGCCAATGAATGG
TATGGAACGTGGAATGGAAGTGCGTGGGCTGATGGTTACAAAAAAGCCAT
TCCGAAACTCCGAAATGCAGGAATTAAAAATACGCTAATTGTGGATGCAG
CCGGATGGGGACAGTTCCCTCAATCCATCGTGGATTATGGACAAAGTGTA
TTTGCAGCCGATTCACAGAAAAATACCGTCTTCTCCATTCATATGTATGA
GTATGCTGGCAAAGATGCTGCAACGGTCAAAGCCAATATGGAGAATGTGC
TGAACAAAGGATTGGCTCTGATCATTGGTGAATTCGGGGGATATCACACA
AACGGTGATGTGGATGAGTATGCCATCATGAGATATGGTCAGGAAAAAGG
GGTAGGCTGGCTTGCCTGGTCTTGGTACGGAAACAGCTCCGGTTTGAACT
ATCTGGACATGGCCACAGGTCCGAACGGAAGCTTAACGAGTTTTGGCAAC
ACTGTTGTTAATGATACCTATGGTATTAAAAACACTTCCCAAAAAGCGGG
GATTTTCTAA.

The amino acid sequence of the protein encoded by the PspMan4 gene, PspMan4 (NCBI Accession No. ACU30843.1) is set forth as SEQ ID NO:12 (the predicted native signal peptide is shown in bold):

MKYLLPTAAAGLLLLAAQPAMAMATGFYVSGNKLYDSTGKPFVMRGVNHG
HSWFKNDLNTAIPAIAKTGANTVRIVLSNGSLYTKDDLNAVKNIINVVNQ
NKMIAVLEVHDATGKDDYNSLDAAVNYWISIKEALIGKEDRVIVNIANEW
YGTWNGSAWADGYKKAIPKLRNAGIKNTLIVDAAGWGQFPQSIVDYGQSV
FAADSQKNTVFSIHMYEYAGKDAATVKANMENVLNKGLALIIGEFGGYHT
NGDVDEYAIMRYGQEKGVGWLAWSWYGNSSGLNYLDMATGPNGSLTSFGN
TVVNDTYGIKNTSQKAGIF.

The nucleic acid sequence for the PspMan5 gene (NCBI Reference Sequence JN603735.1, from 536 to 1519) isolated from Paenibacillus sp. CH-3 is set forth as SEQ ID NO:13 (the sequence encoding the predicted native signal peptide is shown in bold):

ATGAGACAACTTTTAGCAAAAGGTATTTTAGCTGCACTGGTCATGATGTT
AGCGATGTATGGATTGGGGAATCTCTCTTCTAAAGCTTCGGCTGCAACAG
GTTTTTATGTAAGCGGTACCACTCTATATGATTCTACTGGTAAACCTTTT
GTAATGCGCGGTGTCAATCATTCGCATACCTGGTTCAAAAATGATCTAAA
TGCAGCCATCCCTGCTATTGCCAAAACAGGTGCAAATACAGTACGTATCG
TTTTATCTAATGGTGTTCAGTATACTAGAGATGATGTAAACTCAGTCAAA
AATATTATTTCCCTGGTTAACCAAAACAAAATGATTGCTGTTCTTGAGGT
GCATGATGCTACCGGTAAAGACGATTACGCTTCTCTTGATGCCGCTGTAA
ACTACTGGATCAGCATCAAAGATGCCTTGATTGGCAAGGAAGATCGAGTC
ATTGTTAATATTGCCAATGAATGGTACGGTACATGGAATGGCAGTGCTTG
GGCAGATGGTTATAAGCAGGCTATTCCCAAACTAAGAAATGCAGGCATCA
AAAACACTTTAATCGTTGATGCCGCCGGCTGGGGACAATGTCCTCAATCG
ATCGTTGATTACGGGCAAAGTGTATTTGCAGCAGATTCGCTTAAAAATAC
AATTTTCTCTATTCACATGTATGAATATGCAGGCGGTACAGATGCGATCG
TCAAAAGCAATATGGAAAATGTACTGAACAAAGGACTTCCTTTGATCATC
GGTGAATTTGGCGGGCAGCATACAAACGGCGATGTAGATGAACATGCAAT
TATGCGTTATGGTCAGCAAAAAGGTGTAGGTTGGCTGGCATGGTCGTGGT
ATGGCAACAATAGTGAACTCAGTTATCTGGATTTGGCTACAGGTCCCGCC
GGTAGTCTGACAAGTATCGGCAATACGATTGTAAATGATCCATATGGTAT
CAAAGCTACCTCGAAAAAAGCGGGTATCTTCTAA.

The amino acid sequence of the protein encoded by the PspMan5 gene, PspMan5 (NCBI Accession No. AEX60762.1) is set forth as SEQ ID NO:14 (the predicted native signal peptide is shown in bold):

MRQLLAKGILAALVMMLAMYGLGNLSSKASAATGFYVSGTTLYDSTGKPF
VMRGVNHSHTWFKNDLNAAIPAIAKTGANTVRIVLSNGVQYTRDDVNSVK
NIISLVNQNKMIAVLEVHDATGKDDYASLDAAVNYWISIKDALIGKEDRV
IVNIANEWYGTWNGSAWADGYKQAIPKLRNAGIKNTLIVDAAGWGQCPQS
IVDYGQSVFAADSLKNTIFSIHMYEYAGGTDAIVKSNMENVLNKGLPLII
GEFGGQHTNGDVDEHAIMRYGQQKGVGWLAWSWYGNNSELSYLDLATGPA
GSLTSIGNTIVNDPYGIKATSKKAGIF.

In addition, mannanases were identified by sequencing the genomes of Paenibacillus amylolyticus DSM11730, DSM15211, and DSM11747, Paenibacillus pabuli DSM3036, Paenibacillus sp. FeL05 (renamed as Paenibacillus hunanensis DSM22170), and Paenibacillus tundrae (Culture Collection DuPont). The entire genomes of these organisms were sequenced by BaseClear (Leiden, The Netherlands) using the Illumina's next generation sequencing technology and subsequently assembled by BaseClear. Contigs were annotated by BioXpr (Namur, Belgium).

The nucleotide sequence of the PamMan2 gene isolated from Paenibacillus amylolyticus is set forth as SEQ ID NO:15 (the identical sequence was found in DSM11730, DSM15211, and DSM11747; the sequence encoding the predicted native signal peptide is shown in bold):

ATGGTTAATCTGAAAAAGTGTACAATCTTCACGGTTATTGCTACACTCAT
GTTCATGGTATTAGGGAGTGCAGCACCCAAAGCATCTGCTGCTACAGGAT
TTTATGTAAGCGGTAACAAGTTATACGATTCCACAGGCAAGGCTTTTGTC
ATGAGAGGTGTTAATCACGGACATTCCTGGTTCAAAAATGATTTGAATAC
CGCTATCCCTGCAATCGCCAAAACAGGTGCCAATACGGTACGCATTGTTC
TTTCGAATGGTAGCCTGTACACCAAAGATGATCTGAACGCTGTTAAAAAT
ATTATTAATGTGGTTAACCAAAATAAAATGATAGCTGTACTCGAGGTGCA
TGACGCCACAGGGAAAGATGACTATAATTCGTTGGATGCGGCAGTGAACT
ACTGGATTAGCATTAAGGAAGCTTTGATTGGCAAAGAAGATCGGGTCATC
GTCAATATCGCCAATGAATGGTATGGAACGTGGAATGGAAGTGCGTGGGC
TGATGGTTACAAAAAAGCCATTCCGAAACTCCGAAATGCGGGAATTAAAA
ATACGCTAATTGTGGATGCAGCCGGATGGGGACAGTTCCCTCAATCCATC
GTGGATTATGGACAAAGTGTATTTGCAACCGATTCTCAGAAAAATACGGT
CTTCTCCATTCATATGTATGAGTATGCTGGCAAAGATGCTGCAACCGTCA
AAGCCAATATGGAAAATGTGCTGAACAAAGGATTGGCTCTGATCATTGGT
GAGTTCGGGGGATACCACACAAACGGTGATGTGGACGAGTATGCCATCAT
GAGATATGGTCAGGAAAAAGGGGTGGGCTGGCTGGCCTGGTCCTGGTATG
GAAACAGTTCTGGTCTGAACTACCTGGACATGGCTACAGGTCCGAACGGA
AGTTTGACGAGCTTCGGAAACACCGTAGTGAATGATACCTATGGAATTAA
AAAAACTTCTCAAAAAGCGGGGATTTTC.

The amino acid sequence of the PamMan2 precursor protein is set forth as SEQ ID NO:16 (the predicted native signal peptide is shown in bold):

MVNLKKCTIFTVIATLMFMVLGSAAPKASAATGFYVSGNKLYDSTGKAFV
MRGVNHGHSWFKNDLNTAIPAIAKTGANTVRIVLSNGSLYTKDDLNAVKN
IINVVNQNKMIAVLEVHDATGKDDYNSLDAAVNYWISIKEALIGKEDRVI
VNIANEWYGTWNGSAWADGYKKAIPKLRNAGIKNTLIVDAAGWGQFPQSI
VDYGQSVFATDSQKNTVFSIHMYEYAGKDAATVKANMENVLNKGLALIIG
EFGGYHTNGDVDEYAIMRYGQEKGVGWLAWSWYGNSSGLNYLDMATGPNG
SLTSFGNTVVNDTYGIKKTSQKAGIF.

The sequence of the fully processed mature PamMan2 protein (297 amino acids) is set forth as SEQ ID NO:17:

ATGFYVSGNKLYDSTGKAFVMRGVNHGHSWFKNDLNTAIPAIAKTGANTV
RIVLSNGSLYTKDDLNAVKNIINVVNQNKMIAVLEVHDATGKDDYNSLDA
AVNYWISIKEALIGKEDRVIVNIANEWYGTWNGSAWADGYKKAIPKLRNA
GIKNTLIVDAAGWGQFPQSIVDYGQSVFATDSQKNTVFSIHMYEYAGKDA
ATVKANMENVLNKGLALIIGEFGGYHTNGDVDEYAIMRYGQEKGVGWLAW
SWYGNSSGLNYLDMATGPNGSLTSFGNTVVNDTYGIKKTSQKAGIF.

The nucleotide sequence of the PpaMan2 gene isolated from Paenibacillus pabuli DSM3036 is set forth as SEQ ID NO:18 (the sequence encoding the predicted native signal peptide is shown in bold):

ATGGTCAAGTTGCAAAAGGGTACGATCATCACCGTCATTGCTGCGCTCAT
TTTGGTTATGTTGGGAAGTGCTGCACCCAAAGCTTCTGCTGCTGCTGGTT
TTTATGTAAGCGGTAACAAGTTGTATGACTCTACGGGTAAAGCTTTTGTC
ATGCGGGGCGTCAACCACAGTCATACCTGGTTCAAGAACGATCTAAACAC
AGCGATACCCGCCATTGCAAAAACAGGTGCGAACACGGTACGTATTGTGC
TCTCCAATGGGACGCAATATACCAAAGATGATTTGAACGCCGTTAAAAAC
ATAATCAACCTGGTGAGTCAGAACAAAATGATCGCAGTGCTCGAAGTACA
TGATGCAACTGGTAAAGATGACTACAATTCGTTGGATGCAGCAGTCAACT
ACTGGATTAGCATCAAGGAAGCTCTGATTGGCAAGGAAGACCGCGTTATC
GTCAATATTGCCAATGAATGGTACGGGACCTGGAACGGCAGTGCCTGGGC
TGACGGGTACAAAAAAGCAATTCCGAAACTGAGAAATGCCGGCATTAAAA
ATACATTAATTGTAGATGCAGCTGGCTGGGGCCAATATCCGCAATCTATT
GTGGACTATGGTCAAAGTGTTTTTGCAGCAGATGCCCAGAAAAATACGGT
TTTCTCCATTCACATGTATGAATATGCAGGTAAAGATGCCGCAACGGTCA
AAGCCAACATGGAAAACGTGCTGAACAAAGGTTTGGCCCTGATCATCGGT
GAGTTTGGTGGATACCACACCAATGGGGACGTCGATGAATATGCAATCAT
GAAATACGGTCAGGAAAAAGGAGTAGGCTGGCTCGCATGGTCCTGGTATG
GGAACAACTCCGATCTCAATTATCTGGATTTGGCTACAGGTCCAAACGGA
ACTTTAACAAGCTTTGGCAACACGGTGGTTTATGACACGTATGGAATTAA
AAACACTTCGGTAAAAGCAGGGATCTAT.

The amino acid sequence of the PpaMan2 precursor protein is set forth as SEQ ID NO:19 (the predicted native signal peptide is shown in italics and bold):

[symbol]

[symbol]

[symbol]

[symbol]

[symbol]

AAGFYVSGNKLYDSTG
KAFVMRGVNHSHTWFKNDLNTAIPAIAKTGANTVRIVLSNGTQYTKDDL
NAVKNIINLVSQNKMIAVLEVHDATGKDDYNSLDAAVNYWISIKEALIG
KEDRVIVNIANEWYGTWNGSAWADGYKKAIPKLRNAGIKNTLIVDAAGW
GQYPQSIVDYGQSVFAADAQKNTVFSIHMYEYAGKDAATVKANMENVLN
KGLALIIGEFGGYHTNGDVDEYAIMKYGQEKGVGWLAWSWYGNNSDLNY
LDLATGPNGTLTSFGNTVVYDTYGIKNTSVKAGIY.

The nucleotide sequence of the PspMan9 gene isolated from Paenibacillus sp. FeL05 is set forth as SEQ ID NO:20 (the sequence encoding the predicted native signal peptide is shown in bold):

GTGTTTATGTTAGCGATGTATGGATGGGCTGGACTGACTGGTCAAGCTTC
AGCTGCTACAGGTTTTTATGTAAGCGGTACCAAATTATACGACTCTACAG
GCAAGCCATTTGTGATGCGTGGTGTGAATCATTCCCACACCTGGTTCAAA
AATGACCTGAATGCAGCGATCCCTGCAATTGCCAAAACAGGCGCCAACAC
GGTACGTATCGTATTATCGAATGGCGTGCAGTACACCAGAGATGATGTAA
ACTCCGTCAAAAATATCATCTCTCTCGTCAACCAGAACAAAATGATCGCA
GTACTGGAGGTTCATGATGCAACAGGCAAGGACGATTACGCTTCGCTCGA
TGCCGCAATCAACTACTGGATCAGCATCAAGGATGCGCTGATCGGTAAAG
AGGATCGCGTTATCGTCAATATTGCCAACGAATGGTATGGCACATGGAAT
GGAAGCGCATGGGCAGATGGCTACAAACAGGCGATTCCAAAGCTCCGTAA
TGCGGGTATAAAAAATACGCTGATTGTTGACGCAGCCGGCTGGGGTCAAT
ATCCACAATCGATCGTTGATTATGGACAAAGTGTATTTGCAGCGGATTCG
TTAAAAAATACGGTTTTCTCGATCCATATGTATGAGTATGCAGGTGGAAC
CGATGCGATGGTCAAAGCCAACATGGAGGGCGTACTCAATAAAGGTCTGC
CACTGATCATTGGTGAATTTGGCGGACAGCACACAAATGGAGACGTGGAT
GAGCTGGCGATCATGCGTTACGGACAACAAAAAGGAGTAGGCTGGCTCGC
CTGGTCCTGGTACGGCAACAATAGTGATCTGAGTTATCTCGATCTAGCGA
CAGGTCCAAATGGTAGCCTGACCACGTTTGGTAATACGGTGGTAAATGAC
ACCAACGGTATCAAAGCCACCTCCAAAAAAGCAGGTATTTTCCAG.

The amino acid sequence of the PspMan9 precursor protein is set forth as SEQ ID NO:21 (the predicted native signal peptide is shown in italics and bold):

[symbol]

[symbol]

[symbol]

ATGFYVSGTKLYDSTGKPFVMRGVNHSHTW
FKNDLNAAIPAIAKTGANTVRIVLSNGVQYTRDDVNSVKNIISLVNQNK
MIAVLEVHDATGKDDYASLDAAINYWISIKDALIGKEDRVIVNIANEWY
GTWNGSAWADGYKQAIPKLRNAGIKNTLIVDAAGWGQYPQSIVDYGQSV
FAADSLKNTVFSIHMYEYAGGTDAMVKANMEGVLNKGLPLIIGEFGGQH
TNGDVDELAIMRYGQQKGVGWLAWSWYGNNSDLSYLDLATGPNGSLTTF
GNTVVNDTNGIKATSKKAGIFQ.

The nucleotide sequence of the PtuMan2 gene isolated from Paenibacillus tundrae is set forth as SEQ ID NO:22 (the sequence encoding the predicted native signal peptide is shown in bold):

ATGGTCAAGTTGCAAAAGTGTACAGTCTTTACCGTAATTGCTGCACTTAT
GTTGGTGATTCTGGCGAGTGCTGCACCCAAAGCGTCTGCTGCTACAGGAT
TTTATGTAAGCGGAGGCAAATTGTACGATTCTACTGGCAAGGCATTTGTT
ATGAGAGGTGTCAATCATGGACATTCATGGTTTAAGAACGACTTGAACAC
GGCTATTCCTGCGATAGCCAAAACAGGTGCCAACACCGTACGGATTGTGC
TCTCCAATGGCGTACAGTACACCAAAGACGATCTGAACTCTGTTAAAAAC
ATCATTAATGTTGTAAGCGTAAACAAAATGATTGCGGTGCTCGAAGTACA
TGATGCAACAGGTAAGGATGACTATAATTCGTTGGATGCAGCGGTGAACT
ACTGGATTAGCATCAAGGAAGCACTCATTGGCAAAGAAGACAGAGTTATC
GTAAATATCGCGAACGAATGGTATGGAACATGGAACGGCAGTGCCTGGGC
TGACGGATACAAAAAAGCAATTCCGAAGCTGAGAAATGCCGGTATTAAAA
ATACATTGATCGTGGATGCAGCGGGCTGGGGGCAGTACCCGCAATCCATC
GTGGATTATGGACAAAGTGTATTTGCAGCGGATTCACAGAAAAACACCGT
ATTCTCGATTCACATGTATGAATATGCCGGTAAAGACGCAGCAACCGTAA
AAGCCAACATGGAAAGCGTATTAAACAAAGGTCTGGCCCTGATCATCGGT
GAATTCGGTGGATATCACACGAACGGGGATGTCGATGAATATGCGATCAT
GAAATATGGTCAGGAAAAAGGGGTAGGCTGGCTCGCATGGTCCTGGTATG
GCAATAGCTCCGATTTGAACTATTTGGACTTGGCTACGGGACCTAACGGA
AGTTTGACTAGCTTTGGAAACACAGTCGTCAACGACACTTATGGAATCAA
AAATACTTCAAAAAAAGCAGGGATCTAC.

The amino acid sequence of the PtuMan2 precursor protein is set forth as SEQ ID NO:23 (the predicted native signal peptide is shown in bold):

MVKLQKCTVFTVIAALMLVILASAAPKASAATGFYVSGGKLYDSTGKAFV
MRGVNHGHSWFKNDLNTAIPAIAKTGANTVRIVLSNGVQYTKDDLNSVKN
IINVVSVNKMIAVLEVHDATGKDDYNSLDAAVNYWISIKEALIGKEDRVI
VNIANEWYGTWNGSAWADGYKKAIPKLRNAGIKNTLIVDAAGWGQYPQSI
VDYGQSVFAADSQKNTVFSIRMYEYAGKDAATVKANMESVLNKGLALIIG
EFGGYHTNGDVDEYAIMKYGQEKGVGWLAWSWYGNSSDLNYLDLATGPNG
SLTSFGNTVVNDTYGIKNTSKKAGIY.

The sequence of the fully processed mature PtuMan2 (303 amino acids) is set forth as SEQ ID NO:24:

ATGFYVSGGKLYDSTGKAFVMRGVNHGHSWFKNDLNTAIPAIAKTGANTV
RIVLSNGVQYTKDDLNSVKNIINVVSVNKMIAVLEVHDATGKDDYNSLDA
AVNYWISIKEALIGKEDRVIVNIANEWYGTWNGSAWADGYKKAIPKLRNA
GIKNTLIVDAAGWGQYPQSIVDYGQSVFAADSQKNTVFSIHMYEYAGKDA
ATVKANMESVLNKGLALIIGEFGGYHTNGDVDEYAIMKYGQEKGVGWLAW
SWYGNSSDLNYLDLATGPNGSLTSFGNTVVNDTYGIKNTSKKAGIY.

### Example 2

### Heterologous Expression of Mannanases

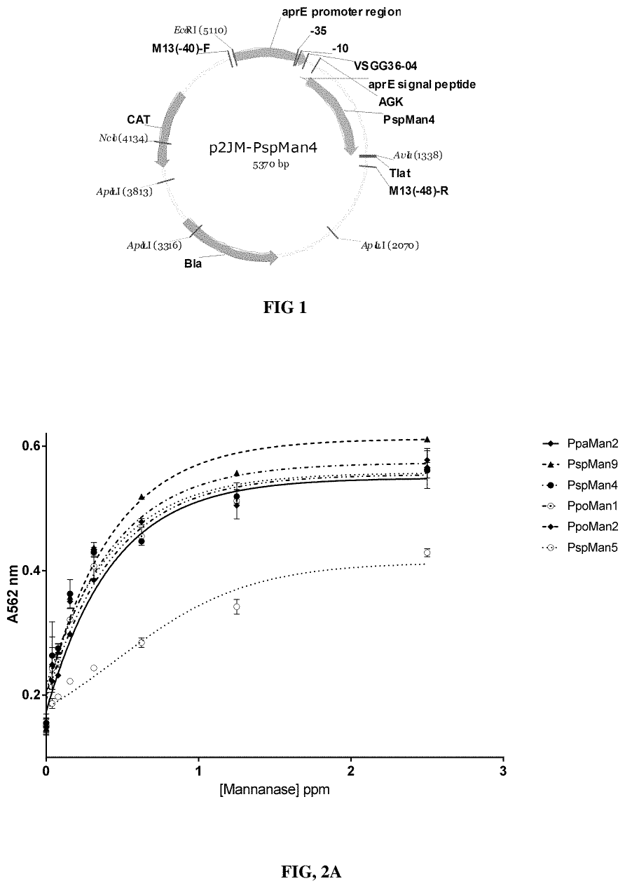

The DNA sequences of the mature forms of BciMan1, BciMan3, BciMan4, PpaMan2, PpoMan1, PpoMan2, PspMan4, PspMan5, and PspMan9 genes were synthesized and inserted into the B. subtilis expression vector p2JM103BBI (Vogtentanz, Protein Expr Purif, 55:40-52, 2007) by Generay Biotech (Shanghai, China), resulting in expression plasmids containing an aprE promoter, an aprE signal sequence used to direct target protein secretion in B. subtilis, an oligonucleotide AGK-proAprE that encodes peptide Ala-Gly-Lys to facilitate the secretion of the target protein, and the synthetic nucleotide sequence encoding the mature region of the gene of interest. A representative plasmid map for PspMan4 expression plasmid (p2JM-PspMan4) is depicted in FIG. 1.

A suitable B. subtilis host strain was transformed with each of the expression plasmids and the transformed cells were spread on Luria Agar plates supplemented with 5 ppm chloramphenicol. To produce each of the mannanases listed above, B. subtilis transformants containing the plasmids were grown in a 250 ml shake flask in a MOPS based defined medium, supplemented with additional 5 mM CaCl₂.

The nucleotide sequence of the synthesized BciMan1 gene in the expression plasmid p2JM-BciMan1 is set forth as SEQ ID NO:25 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAA
GCGGCTTTTATGTTTCAGGCACAAAACTGCTGGATGCAACAGGCCAACCG
TTTGTTATGAGAGGCGTTAATCATGCACATACGTGGTATAAAGATCAACT
GTCAACAGCAATTCCGGCAATCGCAAAAACAGGCGCAAATACAATTAGAA
TTGTTCTGGCGAATGGCCATAAATGGACACTGGATGATGTTAACACAGTC
AACAATATTCTGACACTGTGCGAACAGAATAAACTGATTGCAGTTCTGGA
AGTTCATGATGCGACAGGCTCAGATTCACTGTCAGATCTGGATAATGCAG
TCAATTATTGGATCGGCATTAAATCAGCACTGATCGGCAAAGAAGATCGC
GTCATTATTAACATTGCGAACGAATGGTATGGCACATGGGATGGCGTTGC
ATGGGCAAATGGCTATAAACAAGCGATTCCGAAACTGAGAAATGCAGGCC
TGACACATACACTGATTGTTGATTCAGCAGGCTGGGGACAATATCCGGAT
TCAGTTAAAAACTATGGCACAGAAGTTCTGAACGCAGATCCGCTGAAAAA
TACAGTCTTTAGCATCCACATGTACGAATATGCAGGCGGAAATGCATCAA
CAGTGAAATCAAATATTGATGGCGTCCTGAATAAAAACCTGGCACTGATT
ATTGGCGAATTTGGCGGACAACATACAAATGGCGACGTTGATGAAGCAAC
GATTATGTCATATAGCCAAGAAAAAGGCGTTGGCTGGCTTGCATGGTCAT
GGAAAGGCAATTCATCAGATCTTGCATATCTGGATATGACGAATGATTGG
GCAGGCAATAGCCTGACATCATTTGGCAATACAGTTGTCAATGGCAGCAA
TGGCATTAAAGCAACATCAGTTCTGTCAGGCATTTTTGGCGGAGTTACAC
CGACATCATCACCGACAAGCACACCGACGTCAACACCTACATCAACGCCG
ACACCGACACCTAGCCCGACACCTTCACCGGGAAATAATGGCACAATTCT
GTATGATTTTGAAACAGGCACACAAGGCTGGTCAGGCAATAACATTTCAG
GCGGACCGTGGGTTACAAATGAATGGAAAGCGACAGGCGCACAAACACTG
AAAGCAGATGTTTCACTTCAAAGCAATTCAACGCATAGCCTGTATATCAC
AAGCAATCAAAATCTGAGCGGCAAATCAAGCCTGAAAGCAACAGTTAAAC
ATGCGAATTGGGGCAATATTGGCAATGGAATTTATGCGAAACTGTACGTT
AAAACAGGCAGCGGCTGGACATGGTATGATTCAGGCGAAAATCTGATTCA
GTCAAACGATGGAACAATCCTGACACTTTCACTTTCAGGCATTAGCAATC
TGAGCAGCGTTAAAGAAATTGGCGTCGAATTTAGAGCAAGCTCAAATAGC
TCAGGCCAAAGCGCAATTTATGTTGATAGCGTTTCACTGCAG.

The amino acid sequence of the BciMan1 precursor protein expressed from the p2JM-BciMan1 plasmid is set forth as SEQ ID NO:26 (the predicted signal sequence is shown in italics, the three residue amino-terminal extension (AGK) is shown in bold):

MRSKKLWISLLFALTLIFTMAFSNMSAQAAGKASGFYVSGTKLLDATGQ
PFVMRGVNHAHTWYKDQLSTAIPAIAKTGANTIRIVLANGHKWTLDDVNT
VNNILTLCEQNKLIAVLEVHDATGSDSLSDLDNAVNYWIGIKSALIGKED
RVIINIANEWYGTWDGVAWANGYKQAIPKLRNAGLTHTLIVDSAGWGQYP
DSVKNYGTEVLNADPLKNTVFSIHMYEYAGGNASTVKSNIDGVLNKNLAL
IIGEFGGQHTNGDVDEATEVISYSQEKGVGWLAWSWKGNSSDLAYLDMTN
DWAGNSLTSFGNTVVNGSNGIKATSVLSGIFGGVTPTSSPTSTPTSTPTS
TPTPTPSPTPSPGNNGTILYDFETGTQGWSGNNISGGPWVTNEWKATGAQ
TLKADVSLQSNSTHSLYITSNQNLSGKSSLKATVKHANWGNIGNGIYAKL
YVKTGSGWTWYDSGENLIQSNDGTILTLSLSGISNLSSVKEIGVEFRASS
NSSGQSAIYVDSVSLQ.

The amino acid sequence of the BciMan1 mature protein expressed from p2JM-BciMan1 plasmid is set forth as SEQ ID NO:27 (the three residue amino-terminal extension (AGK) based on the predicted cleavage site shown in bold):

AGKASGFYVSGTKLLDATGQPFVMRGVNHAHTWYKDQLSTAIPAIAKTGA
NTIRIVLANGHKWTLDDVNTVNNILTLCEQNKLIAVLEVHDATGSDSLSD
LDNAVNYWIGIKSALIGKEDRVIINIANEWYGTWDGVAWANGYKQAIPKL
RNAGLTHTLIVDSAGWGQYPDSVKNYGTEVLNADPLKNTVFSIHMYEYAG
GNASTVKSNIDGVLNKNLALIIGEFGGQHTNGDVDEATEVISYSQEKGVG
WLAWSWKGNSSDLAYLDMTNDWAGNSLTSFGNTVVNGSNGIKATSVLSGI
FGGVTPTSSPTSTPTSTPTSTPTPTPSPTPSPGNNGTILYDFETGTQGWS
GNNISGGPWVTNEWKATGAQTLKADVSLQSNSTHSLYITSNQNLSGKSSL
KATVKHANWGNIGNGIYAKLYVKTGSGWTWYDSGENLIQSNDGTILTLSL
SGISNLSSVKEIGVEFRASSNSSGQSAIYVDSVSLQ.

The amino acid sequence of the BciMan1 mature protein, based on the predicted cleavage of the naturally occurring sequence, is set forth as SEQ ID NO:28:

ASGFYVSGTKLLDATGQPFVMRGVNHAHTWYKDQLSTAIPAIAKTGANTI
RIVLANGHKWTLDDVNTVNNILTLCEQNKLIAVLEVHDATGSDSLSDLDN
AVNYWIGIKSALIGKEDRVIINIANEWYGTWDGVAWANGYKQAIPKLRNA
GLTHTLIVDSAGWGQYPDSVKNYGTEVLNADPLKNTVFSIHMYEYAGGNA
STVKSNIDGVLNKNLALIIGEFGGQHTNGDVDEATEVISYSQEKGVGWLA
WSWKGNSSDLAYLDMTNDWAGNSLTSFGNTVVNGSNGIKATSVLSGIFGG
VTPTSSPTSTPTSTPTSTPTPTPSPTPSPGNNGTILYDFETGTQGWSGNN
ISGGPWVTNEWKATGAQTLKADVSLQSNSTHSLYITSNQNLSGKSSLKAT
VKHANWGNIGNGIYAKLYVKTGSGWTWYDSGENLIQSNDGTILTLSLSGI
SNLSSVKEIGVEFRASSNSSGQSAIYVDSVSLQ.

The nucleotide sequence of the synthesized BciMan3 gene in the p2JM-BciMan3 plasmid is set forth as SEQ ID NO:29 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAA
CAGGCTTTTATGTCAATGGCACGAAACTGTATGATAGCACAGGCAAAGCA
TTTGTTATGAGAGGCGTTAATCATCCGCATACGTGGTATAAAAACGATCT
GAATGCAGCAATTCCGGCTATTGCACAAACAGGCGCAAATACAGTTAGAG
TTGTTCTGTCAAATGGCAGCCAATGGACAAAAGATGATCTGAATAGCGTC
AACAGCATTATTTCACTGGTTAGCCAACATCAAATGATTGCAGTTCTGGA
AGTTCATGATGCAACGGGCAAAGATGAATATGCATCACTGGAAGCAGCAG
TCGATTATTGGATTTCAATTAAAGGCGCACTGATCGGCAAAGAAGATAGA
GTCATTGTCAATATTGCGAACGAATGGTATGGCAATTGGAATTCATCAGG
CTGGGCAGATGGCTATAAACAAGCGATTCCGAAACTGAGAAATGCAGGCA
TTAAAAACACACTGATTGTTGATGCAGCAGGCTGGGGACAATATCCGCAA
TCAATTGTCGATGAAGGCGCAGCAGTTTTTGCATCAGATCAACTGAAAAA
CACGGTCTTTAGCATCCACATGTATGAATACGCTGGAAAAGATGCAGCAA
CAGTCAAAACAAATATGGATGACGTTCTGAATAAAGGCCTGCCGCTGATT
ATTGGCGAATTTGGCGGATATCATCAAGGCGCAGATGTTGATGAAATTGC
GATTATGAAATACGGCCAGCAAAAAGAGGTTGGCTGGCTTGCATGGTCAT
GGTATGGAAACTCACCGGAACTGAATGATCTGGATCTGGCAGCAGGACCG
TCAGGCAATCTGACAGGATGGGGCAATACAGTTGTTCATGGCACAGATGG
CATTCAACAGACATCAAAAAAAGCAGGCATCTAT.

The amino acid sequence of the BciMan3 precursor protein expressed from the p2JM-BciMan3 plasmid is set forth as SEQ ID NO:30 (the predicted signal sequence is shown in italics, the three residue amino-terminal extension (AGK) is shown in bold):

MRSKKLWISLLFALTLIFTMAFSNMSAQAAGKATGFYVNGTKLYDSTGKA
FVMRGVNHPHTWYKNDLNAAIPAIAQTGANTVRVVLSNGSQWTKDDLNSV
NSIISLVSQHQMIAVLEVHDATGKDEYASLEAAVDYWISIKGALIGKEDR
VIVNIANEWYGNWNSSGWADGYKQAIPKLRNAGIKNTLIVDAAGWGQYPQ
SIVDEGAAVFASDQLKNTVFSIRMYEYAGKDAATVKTNMDDVLNKGLPLI
IGEFGGYHQGADVDEIAIMKYGQQKEVGWLAWSWYGNSPELNDLDLAAGP
SGNLTGWGNTVVHGTDGIQQTSKKAGIY.

The amino acid sequence of the BciMan3 mature protein expressed from p2JM-BciMan3 is set forth as SEQ ID NO:31 (the three residue amino-terminal extension based on the predicted cleavage site shown in bold):

AGKATGFYVNGTKLYDSTGKAFVMRGVNHPHTWYKNDLNAAIPAIAQTGA
NTVRVVLSNGSQWTKDDLNSVNSIISLVSQHQMIAVLEVHDATGKDEYAS
LEAAVDYWISIKGALIGKEDRVIVNIANEWYGNWNSSGWADGYKQAIPKL
RNAGIKNTLIVDAAGWGQYPQSIVDEGAAVFASDQLKNTVFSIHMYEYAG
KDAATVKTNMDDVLNKGLPLIIGEFGGYHQGADVDEIAIMKYGQQKEVGW
LAWSWYGNSPELNDLDLAAGPSGNLTGWGNTVVHGTDGIQQTSKKAGIY.

The amino acid sequence of the BciMan3 mature protein, based on the predicted cleavage of the naturally occurring sequence, is set forth as SEQ ID NO:32:

ATGFYVNGTKLYDSTGKAFVMRGVNHPHTWYKNDLNAAIPAIAQTGANTV
RVVLSNGSQWTKDDLNSVNSIISLVSQHQMIAVLEVHDATGKDEYASLEA
AVDYWISIKGALIGKEDRVIVNIANEWYGNWNSSGWADGYKQAIPKLRNA
GIKNTLIVDAAGWGQYPQSIVDEGAAVFASDQLKNTVFSIHMYEYAGKDA
ATVKTNMDDVLNKGLPLIIGEFGGYHQGADVDEIAIMKYGQQKEVGWLAW
SWYGNSPELNDLDLAAGPSGNLTGWGNTVVHGTDGIQQTSKKAGIY.

The nucleotide sequence of the synthesized BciMan4 gene in the expression plasmid p2JM-BciMan4 is set forth as SEQ ID NO:33 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAA
CAGGCTTTTATGTTAATGGCGGAAAACTGTATGATAGCACAGGCAAACCG
TTTTATATGCGTGGCATTAATCATGGCCATAGCTGGTTTAAAAACGATCT
GAATACAGCGATTCCGGCTATTGCAAAAACAGGCGCAAATACAGTTAGAA
TTGTTCTGTCAAATGGCACGCAGTATACGAAAGATGATCTGAACTCAGTC
AAAAACATCATCAATGTCGTCAACGCGAACAAAATGATTGCAGTTCTGGA
AGTTCATGATGCAACGGGCAAAGATGATTTCAATTCACTGGATGCAGCAG
TCAACTATTGGATCTCAATTAAAGAAGCGCTGATCGGCAAAGAAGATCGC
GTTATTGTTAATATTGCGAACGAATGGTATGGCACATGGAATGGCTCAGC
ATGGGCAGATGGCTACAAAAAAGCAATTCCGAAACTGAGAGATGCAGGCA
TTAAAAACACACTGATTGTTGATGCGGCAGGCTGGGGACAATATCCGCAA
TCAATTGTTGATTATGGCCAAAGCGTTTTTGCAGCAGATAGCCAGAAAAA
TACAGCGTTTAGCATCCACATGTATGAATATGCGGGAAAAGATGCAGCAA
CAGTCAAAAGCAATATGGAAAACGTCCTGAATAAAGGCCTGGCACTGATT
ATTGGCGAATTTGGCGGATATCATACAAATGGCGACGTTGACGAATATGC
GATTATGAAATATGGCCTGGAAAAAGGCGTTGGCTGGCTTGCATGGTCAT
GGTATGGAAATTCATCAGGCCTTAATTATCTGGATCTGGCAACAGGACCG
AATGGCAGCCTGACATCATATGGCAATACAGTTGTCAATGATACGTATGG
CATCAAAAATACGTCACAGAAAGCAGGCATCTTT.

The amino acid sequence of the BciMan4 precursor protein expressed from plasmid p2JM-BciMan4 is set forth as SEQ ID NO:34 (the predicted signal sequence is shown in italics, the three residue amino-terminal extension (AGK) is shown in bold):

MRSKKLWISLLFALTLIFTMAFSNMSAQAAGKATGFYVNGGKLYDSTGKP
FYMRGINHGHSWFKNDLNTAIPAIAKTGANTVRIVLSNGTQYTKDDLNSV
KNIINVVNANKMIAVLEVHDATGKDDFNSLDAAVNYWISIKEALIGKEDR
VIVNIANEWYGTWNGSAWADGYKKAIPKLRDAGIKNTLIVDAAGWGQYPQ
SIVDYGQSVFAADSQKNTAFSIHMYEYAGKDAATVKSNMENVLNKGLALI
IGEFGGYHTNGDVDEYAIMKYGLEKGVGWLAWSWYGNSSGLNYLDLATGP
NGSLTSYGNTVVNDTYGIKNTSQKAGIF.

The amino acid sequence of the BciMan4 mature protein expressed from p2JM-BciMan4 is set forth as SEQ ID NO:35 (the three residue amino-terminal extension based on the predicted cleavage site shown in bold):

AGKATGFYVNGGKLYDSTGKPFYMIRGINHGHSWFKNDLNTAIPAIAKTG
ANTVRIVLSNGTQYTKDDLNSVKNIINVVNANKMIAVLEVHDATGKDDFN
SLDAAVNYWISIKEALIGKEDRVIVNIANEWYGTWNGSAWADGYKKAIPK
LRDAGIKNTLIVDAAGWGQYPQSIVDYGQSVFAADSQKNTAFSIRMYEYA
GKDAATVKSNMENVLNKGLALIIGEFGGYHTNGDVDEYAIMKYGLEKGVG
WLAWSWYGNSSGLNYLDLATGPNGSLTSYGNTVVNDTYGIKNTSQKAGI
F.

The amino acid sequence of the BciMan4 mature protein, based on the predicted cleavage of the naturally occurring sequence, is set forth as SEQ ID NO:36:

ATGFYVNGGKLYDSTGKPFYMRGINHGHSWFKNDLNTAIPAIAKTGANTV
RIVLSNGTQYTKDDLNSVKNIINVVNANKMIAVLEVHDATGKDDFNSLDA
AVNYWISIKEALIGKEDRVIVNIANEWYGTWNGSAWADGYKKAIPKLRDA
GIKNTLIVDAAGWGQYPQSIVDYGQSVFAADSQKNTAFSIHMYEYAGKDA
ATVKSNMENVLNKGLALITGEFGGYHTNGDVDEYAIMKYGLEKGVGWLAW
SWYGNSSGLNYLDLATGPNGSLTSYGNTVVNDTYGIKNTSQKAGIF.

The nucleotide sequence of the synthesized PpaMan2 gene in plasmid p2JM-PpaMan2 is set forth as SEQ ID NO:37 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAG
CAGGCTTTTATGTTTCAGGCAACAAGCTGTATGATTCAACAGGAAAAGCA
TTTGTTATGAGAGGCGTTAATCATTCACATACATGGTTTAAGAACGATCT
TAATACAGCCATTCCGGCAATCGCGAAGACAGGAGCAAATACAGTGAGAA
TTGTTCTTTCAAACGGAACGCAATATACAAAAGATGACCTGAACGCCGTT
AAGAATATCATTAATCTGGTTTCACAAAATAAGATGATTGCAGTTCTGGA
GGTTCATGATGCAACAGGCAAGGATGACTACAATAGCCTGGATGCAGCGG
TCAATTACTGGATTTCAATTAAAGAAGCACTTATTGGCAAAGAGGATAGA
GTTATTGTTAATATCGCAAATGAATGGTATGGAACGTGGAACGGCTCAGC
ATGGGCAGATGGCTACAAAAAAGCAATTCCGAAACTGAGAAATGCAGGAA
TCAAAAATACACTGATTGTTGACGCCGCAGGCTGGGGACAATATCCGCAA
AGCATCGTTGATTATGGCCAAAGCGTTTTTGCCGCAGACGCACAGAAAAA
CACGGTTTTCTCAATTCATATGTACGAGTATGCTGGAAAGGATGCTGCAA
CGGTTAAAGCTAACATGGAAAATGTTCTGAATAAAGGCCTGGCACTGATC
ATTGGCGAATTTGGAGGCTATCACACAAATGGCGATGTTGATGAATACGC
AATTATGAAATATGGACAAGAAAAAGGCGTTGGATGGCTTGCATGGTCAT
GGTACGGAAACAACTCAGACCTTAATTACCTGGACCTGGCTACGGGACCG
AATGGCACACTGACATCATTCGGCAATACGGTCGTTTATGACACGTATGG
CATCAAGAACACGAGCGTGAAAGCCGGCATTTAT.

The amino acid sequence of the PpaMan2 precursor protein expressed from plasmid p2JM-PpaMan2 is set forth as SEQ ID NO:38 (the predicted signal sequence is shown in italics, the three residue amino-terminal extension (AGK) is shown in bold):

MRSKKLWISLLFALTLIFTMAFSNMSAQAAGKAAGFYVSGNKLYDSTGKA
FVMRGVNHSHTWFKNDLNTAIPAIAKTGANTVRIVLSNGTQYTKDDLNAV
KNIINLVSQNKMIAVLEVHDATGKDDYNSLDAAVNYWISIKEALIGKEDR
VIVNIANEWYGTWNGSAWADGYKKAIPKLRNAGIKNTLIVDAAGWGQYPQ
SIVDYGQSVFAADAQKNTVFSIHMYEYAGKDAATVKANMENVLNKGLALI
IGEFGGYHTNGDVDEYAIMKYGQEKGVGWLAWSWYGNNSDLNYLDLATGP
NGTLTSFGNTVVYDTYGIKNTSVKAGIY.

The amino acid sequence of the PpaMan2 mature protein expressed from p2JM-PpaMan2 is set forth as SEQ ID NO:39 (the three residue amino-terminal extension (AGK) based on the predicted cleavage site shown in bold):

AGKAAGFYVSGNKLYDSTGKAFVMRGVNHSHTWFKNDLNTAIPAIAKTGA
NTVRIVLSNGTQYTKDDLNAVKNIINLVSQNKMIAVLEVHDATGKDDYNS
LDAAVNYWISIKEALIGKEDRVIVNIANEWYGTWNGSAWADGYKKAIPKL
RNAGIKNTLIVDAAGWGQYPQSIVDYGQSVFAADAQKNTVFSIHMYEYAG
KDAATVKANMENVLNKGLALITGEFGGYHTNGDVDEYAIMKYGQEKGVGW
LAWSWYGNNSDLNYLDLATGPNGTLTSFGNTVVYDTYGIKNTSVKAGIY.

The amino acid sequence of the PpaMan2 mature protein, based on the predicted cleavage of the naturally occurring sequence, is set forth as SEQ ID NO:40:

AAGFYVSGNKLYDSTGKAFVMRGVNHSHTWFKNDLNTAIPAIAKTGANTV
RIVLSNGTQYTKDDLNAVKNIINLVSQNKMIAVLEVHDATGKDDYNSLDA
AVNYWISIKEALIGKEDRVIVNIANEWYGTWNGSAWADGYKKAIPKLRNA
GIKNTLIVDAAGWGQYPQSIVDYGQSVFAADAQKNTVFSIHMYEYAGKDA
ATVKANMENVLNKGLALIIGEFGGYHTNGDVDEYAIMKYGQEKGVGWLAW
SWYGNNSDLNYLDLATGPNGTLTSFGNTVVYDTYGIKNTSVKAGIY.

The nucleotide sequence of the synthesized PpoMan1 gene in plasmid p2JM-PpoMan1 is set forth as SEQ ID NO:41 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAA
GCGGCTTTTATGTTTCAGGCACAAAACTGTATGATAGCACAGGCAAACCG
TTTGTTATGAGAGGCGTTAATCATGCACATACGTGGTATAAAAACGATCT
GTATACGGCAATTCCGGCTATTGCACAAACAGGCGCAAATACAGTTAGAA
TTGTTCTGAGCAATGGCAACCAGTATACGAAAGATGATATCAACAGCGTC
AAAAACATTATCAGCCTGGTCAGCAACTATAAAATGATTGCAGTTCTGGA
AGTCCATGATGCAACGGGCAAAGATGATTATGCATCACTGGATGCAGCAG
TCAATTATTGGATTAGCATTAAAGATGCGCTGATCGGCAAAGAAGATCGC
GTTATTGTTAATATTGCGAACGAATGGTATGGCTCATGGAATGGCTCAGG
CTGGGCAGATGGCTATAAACAAGCAATTCCGAAACTGAGAAATGCAGGCA
TTAAAAACACACTGATTGTTGATTGCGCAGGCTGGGGACAATATCCGCAA
TCAATTAATGATTTTGGCAAAAGCGTTTTTGCAGCGGATAGCCTGAAAAA
TACAGTCTTTAGCATCCATATGTATGAATTTGCGGGAAAAGATGCACAGA
CAGTCCGCACAAATATTGATAATGTCCTGAATCAAGGCATCCCGCTGATT
ATTGGCGAATTTGGCGGATATCATCAAGGCGCAGATGTTGATGAAACAGA
AATTATGAGATACGGCCAATCAAAAGGCGTTGGCTGGCTTGCATGGTCAT
GGTATGGAAATTCAAGCAATCTGTCATATCTGGATCTGGTTACAGGACCG
AATGGCAATCTTACAGATTGGGGCAAAACAGTTGTTAATGGCTCAAATGG
CATCAAAGAAACGTCAAAAAAAGCAGGCATCTAT.

The amino acid sequence of the PpoMan1 precursor protein expressed from plasmid p2JM-PpoMan1 is set forth as SEQ ID NO:42 (the predicted signal sequence is shown in italics, the three residue amino-terminal extension (AGK) is shown in bold):

MRSKKLWISLLFALTLIFTMAFSNMSAQAAGKASGFYVSGTKLYDSTGKP
FVMRGVNHAHTWYKNDLYTAIPAIAQTGANTVRIVLSNGNQYTKDDINSV
KNIISLVSNYKMIAVLEVHDATGKDDYASLDAAVNYWISIKDALIGKEDR
VIVNIANEWYGSWNGSGWADGYKQAIPKLRNAGIKNTLIVDCAGWGQYPQ
SINDFGKSVFAADSLKNTVFSIHMYEFAGKDAQTVRTNIDNVLNQGIPLI
IGEFGGYHQGADVDETEIMRYGQSKGVGWLAWSWYGNSSNLSYLDLVTGP
NGNLTDWGKTVVNGSNGIKETSKKAGIY.

The amino acid sequence of the PpoMan1 mature protein expressed from p2JM-PpoMan1 is set forth as SEQ ID NO:43 (the three residue amino-terminal extension based on the predicted cleavage site shown in bold):

AGKASGFYVSGTKLYDSTGKPFVMRGVNHAHTWYKNDLYTAIPAIAQTGA
NTVRIVLSNGNQYTKDDINSVKNIISLVSNYKMIAVLEVHDATGKDDYAS
LDAAVNYWISIKDALIGKEDRVIVNIANEWYGSWNGSGWADGYKQAIPKL
RNAGIKNTLIVDCAGWGQYPQSINDFGKSVFAADSLKNTVFSIHMYEFAG
KDAQTVRTNIDNVLNQGIPLIIGEFGGYHQGADVDETEIMRYGQSKGVGW
LAWSWYGNSSNLSYLDLVTGPNGNLTDWGKTVVNGSNGIKETSKKAGIY.

The amino acid sequence of the PpoMan1 mature protein, based on the predicted cleavage of the naturally occurring sequence, is set forth as SEQ ID NO:44:

ASGFYVSGTKLYDSTGKPFVMRGVNHAHTWYKNDLYTAIPAIAQTGANTV
RIVLSNGNQYTKDDINSVKNIISLVSNYKMIAVLEVHDATGKDDYASLDA
AVNYWISIKDALIGKEDRVIVNIANEWYGSWNGSGWADGYKQAIPKLRNA
GIKNTLIVDCAGWGQYPQSINDFGKSVFAADSLKNTVFSIHMYEFAGKDA
QTVRTNIDNVLNQGIPLIIGEFGGYHQGADVDETEIMRYGQSKGVGWLAW
SWYGNSSNLSYLDLVTGPNGNLTDWGKTVVNGSNGIKETSKKAGIY.

The nucleotide sequence of the synthesized PpoMan2 gene in plasmid p2JM-PpoMan2 is set forth as SEQ ID NO:45 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAGCAA
GCGGCTTTTATGTTTCAGGCACAAATCTGTATGATAGCACAGGCAAACCG
TTTGTTATGAGAGGCGTTAATCATGCACATACGTGGTATAAAAACGATCT
GTATACGGCAATTCCGGCAATCGCAAAAACAGGCGCAAATACAGTTAGAA
TTGTTCTGAGCAATGGCAACCAGTATACGAAAGATGATATCAACAGCGTC
AAAAACATTATCAGCCTGGTCAGCAACCATAAAATGATTGCAGTTCTGGA
AGTTCATGATGCAACGGGCAAAGATGATTATGCATCACTGGATGCAGCAG
TCAATTATTGGATTAGCATTAAAGATGCGCTGATCGGCAAAGAAGATCGC
GTTATTGTTAATATTGCGAACGAATGGTATGGCTCATGGAATGGCGGAGG
CTGGGCAGATGGCTATAAACAAGCAATTCCGAAACTGAGAAATGCAGGCA
TTAAAAACACACTGATTGTTGATTGCGCAGGCTGGGGACAATATCCGCAA
TCAATTAATGATTTTGGCAAAAGCGTTTTTGCAGCGGATAGCCTGAAAAA
TACAGTCTTTAGCATCCATATGTATGAATTTGCAGGCAAAGACGTCCAAA
CAGTCCGCACAAATATTGATAATGTCCTGTATCAAGGCCTGCCGCTGATT
ATTGGCGAATTTGGCGGATATCATCAAGGCGCAGATGTTGATGAAACAGA
AATTATGAGATACGGCCAGTCAAAATCAGTTGGCTGGCTTGCATGGTCAT
GGTATGGAAATTCAAGCAATCTGAACTATCTGGATCTGGTTACAGGACCG
AATGGCAATCTTACAGATTGGGGCAGAACAGTTGTTGAAGGCGCTAATGG
AATTAAAGAAACGTCAAAAAAAGCAGGCATTTTT.

The amino acid sequence of the PpoMan2 precursor protein expressed from plasmid p2JM-PpoMan2 is set forth as SEQ ID NO:46 (the predicted signal sequence is shown in italics, the three residue amino-terminal extension (AGK) is shown in bold):

MRSKKLWISLLFALTLIFTMAFSNMSAQAAGKASGFYVSGTNLYDSTGKP
FVMRGVNHAHTWYKNDLYTAIPAIAKTGANTVRIVLSNGNQYTKDDINSV
KNIISLVSNHKMIAVLEVHDATGKDDYASLDAAVNYWISIKDALIGKEDR
VIVNIANEWYGSWNGGGWADGYKQAIPKLRNAGIKNTLIVDCAGWGQYPQ
SINDFGKSVFAADSLKNTVFSIHMYEFAGKDVQTVRTNIDNVLYQGLPLI
IGEFGGYHQGADVDETEIMRYGQSKSVGWLAWSWYGNSSNLNYLDLVTGP
NGNLTDWGRTVVEGANGIKETSKKAGIF.

The amino acid sequence of the PpoMan2 mature protein expressed from p2JM-PpoMan2 is set forth as SEQ ID NO:47 (the three residue amino-terminal extension (AGK) based on the predicted cleavage site shown in bold):

AGKASGFYVSGTNLYDSTGKPFVMRGVNHAHTWYKNDLYTAIPAIAKTGA
NTVRIVLSNGNQYTKDDINSVKNIISLVSNHKMIAVLEVHDATGKDDYAS
LDAAVNYWISIKDALIGKEDRVIVNIANEWYGSWNGGGWADGYKQAIPKL
RNAGIKNTLIVDCAGWGQYPQSINDFGKSVFAADSLKNTVFSIHMYEFAG
KDVQTVRTNIDNVLYQGLPLIIGEFGGYHQGADVDETEIMRYGQSKSVGW
LAWSWYGNSSNLNYLDLVTGPNGNLTDWGRTVVEGANGIKETSKKAGIF.

The amino acid sequence of the PpoMan2 mature protein, based on the predicted cleavage of the naturally occurring sequence, is set forth as SEQ ID NO:48:

ASGFYVSGTNLYDSTGKPFVMRGVNHAHTWYKNDLYTAIPAIAKTGANTV
RIVLSNGNQYTKDDINSVKNIISLVSNHKMIAVLEVHDATGKDDYASLDA
AVNYWISIKDALIGKEDRVIVNIANEWYGSWNGGGWADGYKQAIPKLRNA
GIKNTLIVDCAGWGQYPQSINDFGKSVFAADSLKNTVFSIHMYEFAGKDV
QTVRTNIDNVLYQGLPLIIGEFGGYHQGADVDETEIMRYGQSKSVGWLAW
SWYGNSSNLNYLDLVTGPNGNLTDWGRTVVEGANGIKETSKKAGIF.

The nucleotide sequence of the synthesized PspMan4 gene in plasmid p2JM-PspMan4 is set forth as SEQ ID NO:49 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTAAT
CTTTACGATGGCGTTCAGCAACATGAGCGCGCAGGCTGCTGGAAAAATGG
CGACAGGCTTTTATGTTTCAGGCAACAAACTGTATGATAGCACAGGCAAA
CCGTTTGTTATGAGAGGCGTTAATCATGGCCATAGCTGGTTTAAAAACGA
TCTGAATACAGCGATTCCGGCTATTGCAAAAACAGGCGCAAATACAGTTA
GAATTGTTCTGTCAAATGGCAGCCTGTATACGAAAGATGATCTGAATGCA
GTCAAAAACATCATCAATGTCGTCAACCAGAACAAAATGATTGCAGTTCT
GGAAGTTCATGATGCAACGGGCAAAGATGATTACAATTCACTGGATGCAG
CAGTCAACTATTGGATCTCAATTAAAGAAGCGCTGATCGGCAAAGAAGAT
CGCGTTATTGTTAATATTGCGAACGAATGGTATGGCACATGGAATGGCTC
AGCATGGGCAGATGGCTACAAAAAAGCAATTCCGAAACTGAGAAATGCAG
GCATCAAAAACACACTGATTGTTGATGCGGCAGGCTGGGGACAATTTCCG
CAATCAATTGTTGATTATGGCCAAAGCGTTTTTGCAGCAGATAGCCAGAA
AAATACAGTCTTTAGCATCCATATGTACGAATACGCTGGAAAAGATGCAG
CAACAGTTAAAGCGAATATGGAAAACGTCCTGAATAAAGGCCTGGCACTG
ATTATTGGCGAATTTGGCGGATATCATACAAATGGCGACGTTGATGAATA
TGCGATTATGAGATATGGCCAAGAAAAAGGCGTTGGCTGGCTTGCATGGT
CATGGTATGGAAATTCATCAGGCCTTAACTATCTGGATATGGCAACAGGA
CCGAATGGATCACTGACATCATTTGGCAATACAGTCGTCAATGATACGTA
TGGAATCAAAAATACGAGCCAGAAAGCTGGCATCTTT.

The amino acid sequence of the PspMan4 precursor protein expressed from plasmid p2JM-PspMan4 is set forth as SEQ ID NO:50 (the predicted signal sequence is shown in italics, the three residue amino-terminal extension (AGK) is shown in bold):

MRSKKLWISLLFALTLIFTMAFSNMSAQAAGKMATGFYVSGNKLYDSTGK
PFVMRGVNHGHSWFKNDLNTAIPAIAKTGANTVRIVLSNGSLYTKDDLNA
VKNIINVVNQNKMIAVLEVHDATGKDDYNSLDAAVNYWISIKEALIGKED
RVIVNIANEWYGTWNGSAWADGYKKAIPKLRNAGIKNTLIVDAAGWGQFP
QSIVDYGQSVFAADSQKNTVFSIRMYEYAGKDAATVKANMENVLNKGLAL
IIGEFGGYHTNGDVDEYAIMRYGQEKGVGWLAWSWYGNSSGLNYLDMATG
PNGSLTSFGNTVVNDTYGIKNTSQKAGIF.

The amino acid sequence of the confirmed PspMan4 mature protein expressed from p2JM-PspMan4 is set forth as SEQ ID NO:51 (the three residue amino-terminal extension (AGK) based on the predicted cleavage site shown in bold):

AGKMATGFYVSGNKLYDSTGKPFVMRGVNHGHSWFKNDLNTAIPAIAKTG
ANTVRIVLSNGSLYTKDDLNAVKNIINVVNQNKMIAVLEVHDATGKDDYN
SLDAAVNYWISIKEALIGKEDRVIVNIANEWYGTWNGSAWADGYKKAIPK
LRNAGIKNTLIVDAAGWGQFPQSIVDYGQSVFAADSQKNTVFSIHMYEYA
GKDAATVKANMENVLNKGLALIIGEFGGYHTNGDVDEYAIMRYGQEKGVG
WLAWSWYGNSSGLNYLDMATGPNGSLTSFGNTVVNDTYGIKNTSQKAGI
F.

The amino acid sequence of the confirmed PspMan4 mature protein, based on the predicted cleavage of the naturally occurring sequence, is set forth as SEQ ID NO:52:

MATGFYVSGNKLYDSTGKPFVMRGVNHGHSWFKNDLNTAIPAIAKTGANT
VRIVLSNGSLYTKDDLNAVKNIINVVNQNKMIAVLEVHDATGKDDYNSLD
AAVNYWISIKEALIGKEDRVIVNIANEWYGTWNGSAWADGYKKAIPKLRN
AGIKNTLIVDAAGWGQFPQSIVDYGQSVFAADSQKNTVFSIHMYEYAGKD
AATVKANMENVLNKGLALITGEFGGYHTNGDVDEYAIMRYGQEKGVGWLA
WSWYGNSSGLNYLDMATGPNGSLTSFGNTVVNDTYGIKNTSQKAGIF.

The nucleotide sequence of the synthesized PspMan5 gene in plasmid p2JM-PspMan5 is set forth as SEQ ID NO:53 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTA
ATCTTTACGATGGCGTTCAGCAACATGAGCGCGCAGGCTGCTGGAAAA
GCAACAGGCTTTTATGTTTCAGGCACAACACTGTATGATTCAACAGGC
AAACCGTTTGTTATGAGAGGCGTTAATCATAGCCATACGTGGTTTAAA
AACGATCTGAATGCAGCAATTCCGGCAATCGCAAAAACAGGCGCAAAT
ACAGTTAGAATTGTTCTGTCAAATGGCGTCCAGTATACAAGAGATGAT
GTCAATAGCGTCAAAAACATTATCAGCCTGGTCAACCAGAACAAAATG
ATTGCAGTTCTGGAAGTTCATGATGCGACAGGCAAAGATGATTATGCA
TCACTGGATGCAGCAGTCAATTATTGGATTAGCATTAAAGATGCGCTG
ATCGGCAAAGAAGATCGCGTTATTGTTAATATTGCGAACGAATGGTAT
GGCACATGGAATGGCTCAGCATGGGCAGATGGCTATAAACAAGCGATT
CCGAAACTGAGAAATGCAGGCATTAAAAACACACTGATTGTTGATGCG
GCAGGCTGGGGACAATGTCCGCAATCAATTGTTGATTATGGCCAATCA
GTTTTTGCAGCGGATAGCCTGAAAAACACAATCTTTAGCATCCATATG
TATGAATATGCAGGCGGAACGGATGCAATTGTCAAAAGCAATATGGAA
AACGTCCTGAATAAAGGCCTGCCGCTGATTATTGGCGAATTTGGCGGA
CAACATACAAATGGCGACGTTGATGAACATGCAATTATGAGATATGGC
CAACAAAAAGGCGTTGGCTGGCTTGCATGGTCATGGTATGGAAATAAT
TCAGAACTGAGCTATCTGGATCTGGCAACAGGACCGGCAGGCTCACTG
ACATCAATTGGAAATACAATTGTGAACGATCCGTATGGCATTAAAGCG
ACATCAAAAAAAGCAGGCATTTTT.

The amino acid sequence of the PspMan5 precursor protein expressed from plasmid p2JM-PspMan5 is set forth as SEQ ID NO:54 (the predicted signal sequence is shown in italics, the three residue amino-terminal extension (AGK) is shown in bold):

MRSKKLWISLLFALTLIFTMAFSNMSAQAAGKATGFYVSGTTLYDSTG
KPFVMRGVNHSHTWFKNDLNAAIPAIAKTGANTVRIVLSNGVQYTRDD
VNSVKNIISLVNQNKMIAVLEVHDATGKDDYASLDAAVNYWISIKDAL
IGKEDRVIVNIANEWYGTWNGSAWADGYKQAIPKLRNAGIKNTLIVDA
AGWGQCPQSIVDYGQSVFAADSLKNTIFSIHMYEYAGGTDAIVKSNME
NVLNKGLPLIIGEFGGQHTNGDVDEHAIMRYGQQKGVGWLAWSWYGNN
SELSYLDLATGPAGSLTSIGNTIVNDPYGIKATSKKAGIF.

The amino acid sequence of the PspMan5 mature protein expressed from p2JM-PspMan5 is set forth as SEQ ID NO:55 (the three residue amino-terminal extension (AGK) based on the predicted cleavage site shown in bold):

AGKATGFYVSGTTLYDSTGKPFVMRGVNHSHTWFKNDLNAAIPAIAKT
GANTVRIVLSNGVQYTRDDVNSVKNIISLVNQNKMIAVLEVHDATGKD
DYASLDAAVNYWISIKDALIGKEDRVIVNIANEWYGTWNGSAWADGYK
QAIPKLRNAGIKNTLIVDAAGWGQCPQSIVDYGQSVFAADSLKNTIFS
IHMYEYAGGTDAIVKSNMENVLNKGLPLIIGEFGGQHTNGDVDEHAIM
RYGQQKGVGWLAWSWYGNNSELSYLDLATGPAGSLTSIGNTIVNDPYG
IKATSKKAGIF.

The amino acid sequence of the PspMan5 mature protein, based on the predicted cleavage of the naturally occurring sequence, is set forth as SEQ ID NO:56:

ATGFYVSGTTLYDSTGKPFVMRGVNHSHTWFKNDLNAAIPAIAKTGAN
TVRIVLSNGVQYTRDDVNSVKNIISLVNQNKMIAVLEVHDATGKDDYA
SLDAAVNYWISIKDALIGKEDRVIVNIANEWYGTWNGSAWADGYKQAI
PKLRNAGIKNTLIVDAAGWGQCPQSIVDYGQSVFAADSLKNTIFSIRM
YEYAGGTDAIVKSNMENVLNKGLPLIIGEFGGQHTNGDVDEHAIMRYG
QQKGVGWLAWSWYGNNSELSYLDLATGPAGSLTSIGNTIVNDPYGIKA
TSKKAGIF.

The nucleotide sequence of the synthesized PspMan9 gene in plasmid p2JM-PspMan9 is set forth as SEQ ID NO: 57 (the gene has an alternative start codon (GTG), the oligonucleotide encoding the three residue addition (AGK) is shown in bold):

GTGAGAAGCAAAAAATTGTGGATCAGCTTGTTGTTTGCGTTAACGTTA
ATCTTTACGATGGCGTTCAGCAACATGAGCGCGCAGGCTGCTGGAAAA
GCAACAGGCTTTTATGTTTCAGGAACAAAACTTTATGATAGCACGGGA
AAACCGTTTGTGATGAGAGGCGTTAATCACTCACATACATGGTTTAAG
AATGATCTGAATGCAGCTATCCCTGCGATTGCGAAGACAGGCGCAAAC
ACGGTTAGAATTGTTCTGTCAAACGGCGTTCAATATACGAGAGATGAT
GTTAATTCAGTCAAGAATATCATTTCACTGGTGAATCAAAATAAGATG
ATTGCAGTTCTGGAAGTTCATGATGCTACAGGAAAAGACGATTATGCA
TCACTGGATGCAGCAATTAACTATTGGATTTCAATTAAAGATGCACTG
ATTGGCAAAGAAGATAGAGTTATTGTGAACATTGCAAATGAATGGTAT
GGCACATGGAATGGCTCAGCATGGGCAGATGGATATAAACAAGCTATT
CCTAAACTGAGAAATGCGGGCATCAAAAATACGCTGATCGTGGATGCG
GCTGGCTGGGGCCAATATCCGCAATCAATTGTTGATTACGGCCAGTCA
GTTTTTGCAGCAGATTCACTGAAGAACACAGTGTTTAGCATCCATATG
TATGAATATGCAGGCGGCACAGATGCAATGGTTAAAGCTAATATGGAA
GGAGTTCTGAATAAAGGCCTGCCGCTGATTATTGGAGAATTTGGCGGA
CAACATACAAATGGCGATGTTGACGAACTGGCAATTATGAGATATGGC
CAACAAAAAGGCGTGGGATGGCTGGCATGGTCATGGTACGGCAACAAC
AGCGATCTGTCATATCTTGATCTGGCAACGGGACCGAATGGATCACTG
ACAACGTTTGGAAATACAGTGGTGAACGATACGAACGGAATTAAGGCA
ACGAGCAAGAAGGCGGGAATTTTTCAA.

The amino acid sequence of the PspMan9 precursor protein expressed from plasmid p2JM-PspMan9 is set forth as SEQ ID NO:58 (the predicted signal sequence is shown in italics, the three residue amino-terminal extension (AGK) is shown in bold):

MRSKKLWISLLFALTLIFTMAFSNMSAQAAGKATGFYVSGTKLYDSTG
KPFVMRGVNHSHTWFKNDLNAMPAIAKTGANTVRIVLSNGVQYTRDDV
NSVKNIISLVNQNKMIAVLEVHDATGKDDYASLDAAINYWISIKDALI
GKEDRVIVNIANEWYGTWNGSAWADGYKQAIPKLRNAGIKNTLIVDAA
GWGQYPQSIVDYGQSVFAADSLKNTVFSIHMYEYAGGTDAMVKANMEG
VLNKGLPLIIGEFGGQHTNGDVDELAIMRYGQQKGVGWLAWSWYGNNS
DLSYLDLATGPNGSLTTFGNTVVNDTNGIKATSKKAGIFQ

The amino acid sequence of the PspMan9 mature protein expressed from p2JM-PspMan9 is set forth as SEQ ID NO:59 (the three residue amino-terminal extension (AGK) based on the predicted cleavage site shown in bold):

AGKATGFYVSGTKLYDSTGKPFVMRGVNHSHTWFKNDLNAAIPAIAKT
GANTVRIVLSNGVQYTRDDVNSVKNIISLVNQNKMIAVLEVHDATGKD
DYASLDAAINYWISIKDALIGKEDRVIVNIANEWYGTWNGSAWADGYK
QMPKLRNAGIKNTLIVDAAGWGQYPQSIVDYGQSVFAADSLKNTVFSI
HMYEYAGGTDAMVKANMEGVLNKGLPLIIGEFGGQHTNGDVDELAIMR
YGQQKGVGWLAWSWYGNNSDLSYLDLATGPNGSLTTFGNTVVNDTNGI
KATSKKAGIFQ.

The amino acid sequence of the PspMan9 mature protein, based on the predicted cleavage of the naturally occurring sequence, is set forth as SEQ ID NO:60:

ATGFYVSGTKLYDSTGKPFVMRGVNHSHTWFKNDLNAAIPAIAKTGAN
TVRIVLSNGVQYTRDDVNSVKNIISLVNQNKMIAVLEVHDATGKDDYA
SLDAAINYWISIKDALIGKEDRVIVNIANEWYGTWNGSAWADGYKQMP
KLRNAGIKNTLIVDAAGWGQYPQSIVDYGQSVFAADSLKNTVFSIHMY
EYAGGTDAMVKANMEGVLNKGLPLIIGEFGGQHTNGDVDELAIMRYGQ
QKGVGWLAWSWYGNNSDLSYLDLATGPNGSLTTFGNTVVNDTNGIKAT
SKKAGIFQ.

### Example 3

### Purification of Mannanases

BciMan1, BciMan4, and PspMan4 proteins were purified via two chromatography steps: anion-exchange and hydrophobic interaction chromatography. The concentrated and desalted crude protein samples were loaded onto a 70 ml Q-Sepharose High Performance column pre-equilibrated with buffer A (Tris-HCl, pH7.5, 20 mM). After column washing, the proteins were eluted with a gradient of 0-50% buffer A with 1 M NaCl in 5 column volumes. The target protein was in the flowthrough. Ammonium sulfate was then added to the flowthrough to a final concentration of 0.8-1 M. The solution was loaded onto a Phenyl-Sepharose Fast Flow column pre-equilibrated with 20 mM Tris pH 7.5 with 0.8-1 M ammonium sulfate (buffer B). Gradient elution (0-100% buffer A) in 4 column volumes followed with 3 column volumes step elution (100% buffer A) was performed and the protein of interest was eventually eluted. The purity of the fractions was detected with SD S-PAGE and the results showed that the target protein had been completely purified. The active fractions were pooled and concentrated using 10 kDa Amicon Ultra-15 devices. The sample was above 90% pure and stored in 40% glycerol at −20° C. to −80° C. until usage.

BciMan3, PpoMan1, PpoMan2 proteins were purified using a three step anion-exchange, hydrophobic interaction chromatography and gel filtration purification strategy. The 700 mL crude broth from the shake flask was concentrated by VIVAFLOW 200 (cutoff 10 kDa) and buffer exchanged into 20 mM Tris-HCl (pH 7.5). The liquid was then loaded onto a 50 mL Q-Sepharose High Performance column which was pre-equilibrated with 20 mM Tris-HCl, pH 7.5 (buffer A). The column was eluted with a linear gradient from 0 to 50% buffer B (buffer A containing 1 M NaCl) in 3 column volumes, followed with 3 column volumes of 100% buffer B. The protein of interest was detected in the gradient elution part and the pure fractions were pooled. Subsequently, 3 M ammonium sulfate solution was added to the active fractions to an ultimate concentration of 1 M, and then the pretreated fraction was loaded onto a 50 mL Phenyl-Sepharose Fast Flow column equilibrated with 20 mM Tris-HCl (pH 7.5) containing 1 M ammonium sulfate. Four column volumes gradient elution (0-100% buffer A) followed with 3 column volumes step elution (100% buffer A) was performed and the relative pure fractions were pooled. The collected fraction was concentrated into 10 mL and loaded onto the HiLoad™ 26/60, Superdex-75 column (1 column volume=320 mL) pre-equilibrated with 20 mM sodium phosphate buffer containing 0.15 M NaCl (pH 7.0). The pure fractions were pooled and concentrated using 10 kDa Amicon Ultra-15 devices. The purified sample was stored in 20 mM sodium phosphate buffer (pH 7.0) with 40% glycerol at −20° C. until usage.

To purify PspMan5 and PspMan9 proteins, ammonium sulfate was added to the crude samples to a final concentration of 1 M. The solution was applied to a HiPrep™ 16/10 Phenyl FF column pre-equilibrated with 20 mM Tris (pH 8.5), 1M ammonium sulfate (buffer A). The target protein was eluted from the column with a linear salt gradient from 1 to 0 M ammonium sulfate. The active fractions were pooled, concentrated and buffer exchanged into 20 mM Tris (pH8.5) using a VivaFlow 200 ultra filtration device (Sartorius Stedim). The resulting solution was applied to a HiPrep™ Q XL 16/10 column pre-equilibrated with 20 mM Tris (pH8.5). The target protein was eluted from the column with a linear salt gradient from 0 to 0.6 M NaCl in buffer A. The resulting active protein fractions were then pooled and concentrated via 10 kDa Amicon Ultra devices, and stored in 40% glycerol at −20° C. until usage.

PpaMan2 was purified using hydrophobic interaction chromatography and cation exchange chromatography. 800 mL crude broth was concentrated by VIVAFLOW 200 (cutoff 10 kDa) and ammonium sulfate was added to a final concentration of 0.8 M. The sample was then loaded onto a 50 mL Phenyl-Sepharose High Performance column which was pre-equilibrated with buffer A (20 mM sodium acetate containing 0.8 M ammonium sulfate, pH 5.5). The column was treated with a gradient elution of 0-100% buffer B (20 mM sodium acetate at pH 5.5) in 5 column volumes, followed with 3 column volumes of 100% buffer B. The relative pure active fractions were pooled and buffer exchanged into buffer B. The solution turned to be cloudy and was dispensed to 50 mL tubes, centrifuged at 3800 rpm for 20 min. The supernatant and the precipitant were collected. According to the SDS-PAGE gel analysis results, the target protein was identified in the supernatant which was then subjected onto an SP-Sepharose Fast Flow column, a linear gradient elution with 0-50% buffer C (20 mM sodium acetate containing 1M sodium chloride) in 4 column volumes followed with 3 column volumes' step elution (100% buffer C) was performed. The purity of the each fraction was evaluated with SDS-PAGE. Pure fractions were pooled and concentrated using 10 kDa Amicon Ultra-15 devices. The purified sample was stored in 20 mM sodium acetate buffer (pH 5.5) with 40% glycerol at −20° C.

### Example 4

### Activity of Mannanases

The beta 1-4 mannanase activity of the mannanases was measured using 0.5% locust bean gum galactomannan (Sigma G0753) and konjac glucomannan (Megazyme P-GLCML) as substrates. The assays were performed at 50° C. for 10 minutes using two different buffer systems: 50 mM sodium acetatepH 5, and 50 mM HEPES pH 8.2. In both sets of assays, the released reducing sugar was quantified using a PAHBAH (p-Hydroxy benzoic acid hydrazide) assay (Lever, Anal Biochem, 47:248, 1972). A standard curve using mannose was created for each buffer, and was used to calculate enzyme activity units. In this assay, one mannanase unit is defined as the amount of enzyme required to generate 1 micromole of mannose reducing sugar equivalent per minute. The specific activities of the mannanases are summarized in Table 1.

TABLE 1
Specific activities (U/mg) of mannanases at
pH 5.0 and pH 8.2 using different substrates

| | pH 5.0 | | pH 8.2 | |
|---|---|---|---|---|
| | Locust bean | Konjac | Locust bean | Konjac |
| Mannanase | gum | glucomannan | gum | glucomannan |
| BciMan1 | 25 | 70 | 328 | 363 |
| BciMan3 | 17 | 35 | 377 | 414 |
| BciMan4 | 160 | 221 | 590 | 681 |
| PpaMan2 | 94 | 162 | 419 | 454 |
| PpoMan1 | 148 | 205 | 616 | 601 |
| PpoMan2 | 62 | 108 | 618 | 615 |
| PspMan4 | 112 | 159 | 520 | 624 |
| PspMan5 | 105 | 136 | 116 | 152 |
| PspMan9 | 145 | 251 | 518 | 628 |

### Example 5

### pH Profile of Mannanases

The pH profile of mannanases was determined by assaying for mannanase activity at various pH values ranging from 2 to 9 at 50° C. for 10 min with locust bean gum as the substrate. The proteins were diluted in 0.005% Tween-80 to an appropriate concentration based on the dose response curve. The substrate solutions, buffered using sodium citrate/sodium phosphate buffers of different pH units, were pre-incubated in the thermomixer at 50° C. for 5 min. The reaction was initiated by the addition of mannanases. The mixture was incubated at 50° C. for 10 min, and then the reaction was stopped by transferring 10 microliters of reaction mixture to a 96-well PCR plate containing 100 microliters of the PAHBAH solution. The PCR plate was heated at 95° C. for 5 minutes in a Bio-Rad DNA Engine. Then 100 microliters were transferred from each well to a new 96-well plate. The release of reducing sugars from the substrate was quantified by measuring the optical density at 410 nm in a spectrophotometer. Enzyme activity at each pH is reported as relative activity where the activity at the pH optimum was set to 100%. The pH optimum and range of ≥70% activity for the mannanases under these assay conditions is shown in Table 2.

TABLE 2
Optimal pH and pH range of activity for mannanases

| | | pH range |
|---|---|---|
| | pH | of ≥70% |
| Mannanase | Optimum | activity |
| BciMan1 | 7.0 | 6.0-8.5 |
| BciMan3 | 7.0 | 6.5-8.5 |
| BciMan4 | 7.0 | 5.5-8.5 |
| PpaMan2 | 8.0 | 5.5-9.0* |
| PpoMan1 | 7.0 | 5.5-8.5 |
| PpoMan2 | 7.0 | 6.0-8.5 |
| PspMan4 | 7.5 | 5.5-9.0 |
| PspMan5 | 6.0 | 4.5-7.5 |
| PspMan9 | 6.0-8.0 | 5.5-9.0* |

*PpaMan2 and PspMan9 showed mannanase activity above pH 9

### Example 6

### Temperature Profile of Mannanases

The temperature profile of mannanases was determined by assaying for mannanase activity with locust bean gum as the substrate at various temperatures for 10 min in 50 mM sodium citrate buffer at pH 6.0. The activity is reported as relative activity where the activity at the temperature optimum was set to 100%. The temperature optimum and temperature range of ≥70% activity for the mannanases under these assay conditions is shown in Table 3.

TABLE 3
Optimal temperature and temperature
range of activity for mannanases.

| | Temperature | Temperature range |
|---|---|---|
| | Optimum | of ≥70% activity |
| Mannanase | (° C.) | (° C.) |
| BciMan1 | 60-65 | 45-70 |
| BciMan3 | 55 | 40-65 |
| BciMan4 | 55 | 50-60 |
| PpaMan2 | 60 | 54-63 |
| PpoMan1 | 55-58 | 45-65 |
| PpoMan2 | 50-55 | <35-60 |
| PspMan4 | 55 | 47-60 |
| PspMan5 | 50 | 40-55 |
| PspMan9 | 58 | 48-62 |

### Example 7

### Thermostability of Paenibacillus and Bacillus Mannanases

The temperature stability of Paenibacillus and Bacillus mannanases was determined in 50 mM sodium citrate buffer at pH 6.0. The enzyme was incubated at temperatures ranging from 40° C. to 90° C. for 2 hours in a thermocycler. The remaining enzyme activity was measured using locust bean gum as the substrate. The activity of the sample kept on ice was defined as 100% activity. The temperatures at which the enzymes retain 50% activity (T₅₀) after a 2-hour incubation period under these assay conditions are shown in Table 4.

TABLE 4
Thermal Stability of Mannanases.

| | T₅₀ |
|---|---|
| Mannanase | (° C.) |
| PspMan4 | 57 |
| BciMan1 | 53 |
| BciMan3 | 47 |
| BciMan4 | 53 |
| PpoMan1 | 54 |
| PpoMan2 | 52 |
| PspMan5 | 53 |
| PspMan9 | 54 |
| PpaMan2 | 58 |

### Example 8

### Cleaning Performance of Mannanases

Cleaning performance was measured using a high throughput assay developed to measure galactomannan removal from technical soils. The assay measures the release of locust bean gum from the technical soils containing locust bean gum. The BCA reagent measures the reducing ends of oligosaccharides released in the presence of mannanase enzyme, as compared to a blank (no enzyme) control. This measurement correlates with the cleaning performance for the enzymes. As the mannanases hydrolyze galactomannans, oligosaccharides of varying lengths with new reducing ends are released from the cotton swatch. The bicinchoninic acid in the BCA reagent then allows for the highly sensitive colorimetric detection as Cu′ is formed by the reduction of Cu²⁺.

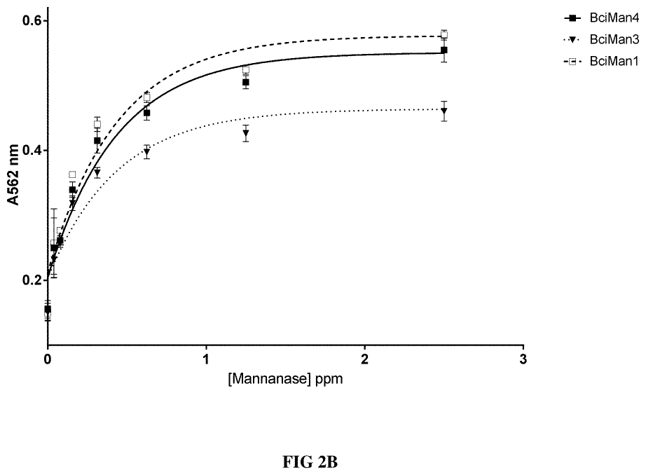

Two 5.5 cm diameter locust bean gum CS-73 microswatches (CFT, Vlaardingen, Holland) were placed into each well of a flat-bottom, non-binding 96-well assay plate. Enzymes were diluted into 50 mM MOPS, pH 7.2, 0.005% Tween-80. Diluted enzyme and microswatch assay buffer (25 mM HEPES, pH 8, 2 mM CaCl₂, 0.005% Tween-80) was added into each well for a combined volume of 100 microliters. Plates were sealed and incubated in an iEMS machine at 25° C. with agitation at 1150 rpm for 20 minutes. To measure the new reducing ends produced, 100 microliters of the BCA assay reagent (Thermo Scientific Pierce, Rockford, Ill.) was pipetted into each well of a fresh PCR plate. 15 microliters of wash liquor was removed from each well of the microswatch assay plates after the incubation period was completed, and transferred to the plate containing the BCA reagent. Plates were sealed and incubated in a PCR machine at 95° C. for 2-3 minutes. After the plate cooled to 25° C., 100 microliters of the supernatant was transferred to a fresh microtiter flat-bottom assay plate and absorbance was measured at 562 nm in a spectrophotometer. FIGS. 2A and 2B show the response of the mannanases in this assay. All mannanases tested exhibited galactomannan removal activity.

### Example 9

### Identification of Homologous Mannanases

Related proteins were identified by a BLAST search (Altschul et al., Nucleic Acids Res, 25:3389-402, 1997) against the NCBI non-redundant protein database using the mature protein amino acid sequence of PpaMan2 (SEQ ID NO:40), PspMan4 (SEQ ID NO:52), and PspMan9 (SEQ ID NO:60) and a subset of the results are shown on Tables 5A, 6A, and 7A, respectively. A similar search was run against the Genome Quest Patent database with search parameters set to default values using the mature protein amino acid sequence of PpaMan2 (SEQ ID NO:40), PspMan4 (SEQ ID NO:52), and PspMan9 (SEQ ID NO:60) as the query sequences, and a subset of the results are shown in Tables 5B, 6B, and 7B, respectively. Percent identity (PID) for both search sets is defined as the number of identical residues divided by the number of aligned residues in the pairwise alignment. The column labeled “Sequence Length” refers to the length (in amino acids) of the protein sequences associated with the listed Accession Nos., while the column labeled “Aligned Length” refers to the length (in amino acids) of the aligned protein sequence used for the PID calculation.

TABLE 5A
List of sequences with percent identity to PpaMan2 protein
identified from the NCBI non-redundant protein database

| | | | Sequence | Alignment |
|---|---|---|---|---|
| Accession # | PID | Organism | Length | Length |
| WP_024633848.1 | 95 | Paenibacillus sp. MAEPY2] | 326 | 296 |
| ETT37549.1 | 94 | Paenibacillus sp. FSL R5-192 | 326 | 296 |
| WP_017688745.1 | 93 | Paenibacillus sp. PAMC 26794 | 326 | 296 |
| ACU30843.1 | 93 | Paenibacillus sp. A1 | 319 | 296 |
| AAX87003.1 | 91 | B. circulans | 326 | 296 |
| WP_017813111.1 | 88 | Paenibacillus sp. A9 | 327 | 296 |
| AEX60762.1 | 86 | Paenibacillus sp. CH-3 | 327 | 296 |
| YP_003868989.1/WP_013308634.1 | 81 | Paenibacillus polymyxa E681 | 327 | 296 |
| WP_016819573.1 | 81 | Paenibacillus polymyxa | 327 | 296 |
| WP_017427981.1 | 81 | Paenibacillus sp. ICGEB2008 | 327 | 296 |
| YP_003944884.1/WP_013369280.1 | 80 | Paenibacillus polymyxa SC2 | 327 | 296 |
| WP_009593769.1 | 80 | Paenibacillus sp. HGF5 | 326 | 296 |
| AAX87002.1 | 81 | B. circulans | 327 | 296 |
| BAA25878.1 | 71 | B. circulans | 516 | 297 |
| WP_019912481.1 | 66 | Paenibacillus sp. HW567 | 547 | 294 |
| YP_006190599.1/WP_014651264.1 | 66 | Paenibacillus mucilaginosus K02 | 475 | 296 |

TABLE 5B
List of sequences with percent identity to PpaMan2
protein identified from the Genome Quest database

| | | | Sequence | Alignment |
|---|---|---|---|---|
| Patent ID # | PID | Organism | Length | Length |
| EP2260105-0418 | 91.6 | B. circulans | 326 | 296 |
| EP2260105-0427 | 81.1 | B. circulans | 327 | 296 |
| CN100410380-0004, | 81.1 | B. circulansB48 | 296 | 296 |
| CN1904052-0003 | 80.4 | B. circulansB48 | 327 | 296 |
| US20090325240-0477 | 71.7 | B. circulans | 516 | 297 |
| US20140199705-0388 | 68.4 | empty | 490 | 291 |
| WO2014100018-0002 | 66 | Bacillus lentus | 299 | 297 |
| WO2015022428-0015 | 63.1 | Bacillus sp. | 309 | 290 |
| US20030203466-0004 | 62.8 | Bacillus sp. | 490 | 290 |
| EP2260105-0445 | 62.1 | B. circulans | 493 | 290 |
| EP2260105-0429 | 61.8 | Bacillus sp. | 490 | 296 |
| | | JAMB-602 | | |
| US20030215812-0002 | 60.6 | Bacillus sp. | 493 | 297 |
| US20030203466-0008 | 60.6 | Bacillus | 468 | 297 |
| | | agaradhaerens | | |
| US20030215812-0002 | 60.6 | Bacillus sp | 493 | 297 |

TABLE 6A
List of sequences with percent identity to PspMan4 protein
identified from the NCBI non-redundant protein database

| | | | Sequence | Alignment |
|---|---|---|---|---|
| Accession # | PID | Organism | Length | Length |
| ACU30843.1 | 100 | Paenibacillus sp. A1 | 319 | 297 |
| ETT37549.1 | 99 | Paenibacillus sp. FSL R5-192 | 326 | 296 |
| WP_017688745.1 | 99 | Paenibacillus sp. PAMC 26794 | 326 | 296 |
| AAX87003.1 | 94 | B. circulans | 326 | 296 |
| WP_024633848.1 | 94 | Paenibacillus sp. MAEPY2 | 326 | 296 |
| WP_017813111.1 | 89 | Paenibacillus sp. A9 | 327 | 296 |
| AEX60762.1 | 87 | Paenibacillus sp. CH-3 | 327 | 296 |
| YP_003868989.1/WP_013308634.1 | 81 | Paenibacillus polymyxa E681 | 327 | 296 |
| YP_003944884.1/WP_013369280.1 | 80 | Paenibacillus polymyxa SC2 | 327 | 296 |
| WP_016819573.1 | 80 | Paenibacillus polymyxa | 327 | 296 |
| WP_017427981.1 | 80 | Paenibacillus sp. ICGEB2008 | 327 | 296 |
| AAX87002.1 | 79 | B. circulans | 327 | 296 |
| WP_009593769.1 | 78 | Paenibacillus sp. HGF5 | 326 | 296 |
| BAA25878.1 | 72 | B. circulans | 516 | 297 |
| YP_006190599.1/WP_014651264.1 | 67 | Paenibacillus mucilaginosus K02 | 475 | 296 |
| WP_019912481.1 | 65 | Paenibacillus sp. HW567 | 547 | 294 |
| BAD99527.1 | 62 | Bacillus sp. JAMB-602 | 490 | 296 |
| AGU71466.1 | 64 | Bacillus nealsonii | 353 | 297 |
| WP_017426982.1 | 63 | Paenibacillus sp. ICGEB2008 | 796 | 296 |
| AAS48170.1 | 61 | Bacillus circulans | 493 | 296 |
| AAT06599.1 | 60 | Bacillus sp. N16-5 | 493 | 297 |
| WP_018887458.1 | 63 | Paenibacillus massiliensis | 592 | 294 |
| YP_006844719.1 | 60 | Amphibacillus xylanus NBRC 15112 | 497 | 297 |

TABLE 6B
List of sequences with percent identity to PspMan4
protein identified from the Genome Quest database

| | | | Sequence | Alignment |
|---|---|---|---|---|
| Patent ID # | PID | Organism | Length | Length |
| EP2260105-0418 | 94.3 | B. circulans | 326 | 296 |
| CN100410380-0004 | 79.1 | B. circulansB48 | 296 | 296 |
| EP2260105-0427 | 79.1 | B. circulans | 327 | 296 |
| CN1904052-0003 | 78.4 | B. circulansB48 | 327 | 296 |
| US20090325240-0477 | 72.1 | B. circulans | 516 | 297 |
| EP2409981-0388 | 67.7 | empty | 490 | 297 |
| WO2014100018-0002 | 66.3 | Bacillus lentus | 299 | 297 |
| WO2015022428-0015 | 62.5 | Bacillus sp. | 309 | 296 |
| JP2006087401-0006 | 62.5 | Bacillus sp. | 458 | 296 |
| US20090325240-0429 | 62.5 | Bacillus sp. | 490 | 296 |
| | | JAMB-602 | | |
| EP2284272-0004 | 62.2 | Bacillus sp. | 476 | 296 |
| EP2287318-0002 | 62.2 | Bacillus sp. I633 | 490 | 296 |
| WO2014124927-0018 | 62.2 | Bacillus sp. I633 | 490 | 296 |
| US20090325240-0445 | 61.5 | B. circulans | 493 | 296 |
| US20030203466-0008 | 60.9 | Bacillus | 468 | 297 |
| | | agaradhaerens | | |
| US6964943-0002 | 60.9 | Bacillus sp. | 493 | 297 |

TABLE 7A
List of sequences with percent identity to PspMan9 protein
identified from the NCBI non-redundant protein database

| | | | Sequence | Alignment |
|---|---|---|---|---|
| Accession # | PID | Organism | Length | Length |
| AEX60762.1 | 94 | Paenibacillus sp. CH-3 | 327 | 296 |
| WP_017813111.1 | 89 | Paenibacillus sp. A9 | 327 | 296 |
| ACU30843.1 | 88 | Paenibacillus sp. A1 | 319 | 297 |
| WP_024633848.1 | 88 | Paenibacillus sp. MAEPY2] | 326 | 296 |
| ETT37549.1 | 88 | Paenibacillus sp. FSL R5-192 | 326 | 296 |
| WP_017688745.1 | 87 | Paenibacillus sp. PAMC 26794 | 326 | 296 |
| AAX87003.1 | 86 | B. circulans | 326 | 296 |
| YP_003868989.1/WP_013308634.1 | 83 | Paenibacillus polymyxa E681 | 327 | 296 |
| WP_016819573.1 | 83 | Paenibacillus polymyxa | 327 | 296 |
| WP_017427981.1 | 82 | Paenibacillus sp. ICGEB2008 | 327 | 296 |
| YP_003944884.1/WP_013369280.1 | 82 | Paenibacillus polymyxa SC2 | 327 | 296 |
| AAX87002.1 | 80 | B. circulans | 327 | 296 |
| WP_009593769.1 | 79 | Paenibacillus sp. HGF5 | 326 | 296 |
| BAA25878.1 | 73 | B. circulans | 516 | 297 |
| YP_006190599.1/WP_014651264.1 | 68 | Paenibacillus mucilaginosus K02 | 475 | 296 |
| WP_019912481.1 | 66 | Paenibacillus sp. HW567 | 547 | 294 |
| AGU71466.1 | 68 | B. nealsonii | 353 | 297 |
| WP_018887458.1 | 65 | Paenibacillus massiliensis | 592 | 294 |
| WP_019687326.1 | 64 | Paenibacillus polymyxa | 796 | 296 |
| WP_006037399.1 | 64 | Paenibacillus curdlanolyticus | 707 | 297 |

TABLE 7B
List of sequences with percent identity to PspMan9
protein identified from the Genome Quest database

| | | | Sequence | Alignment |
|---|---|---|---|---|
| Patent ID # | PID | Organism | Length | Length |
| EP2260105-0418 | 86.2 | B. circulans | 326 | 296 |
| CN100410380-0004 | 80.4 | B. circulansB48 | 296 | 296 |
| EP2260105-0427 | 80.4 | B. circulans | 327 | 296 |
| CN1904052-0003 | 79.7 | B. circulansB48 | 327 | 296 |
| EP2260105-0477 | 73.4 | B. circulans | 516 | 297 |
| US20140199705-0388 | 68.4 | empty | 490 | 297 |
| WO2014100018-0002 | 68 | Bacillus lentus | 299 | 297 |
| JP2006087401-0001 | 62.8 | Bacillus sp. | 458 | 296 |
| WO2015022428-0015 | 62.5 | Bacillus sp. | 309 | 296 |
| US20030203466-0004 | 62.2 | Bacillus sp. | 490 | 296 |
| JP2006087401-0005 | 62.8 | Bacillus sp. | 490 | 296 |
| US20090325240-0429 | 62.8 | Bacillus sp. | 490 | 296 |
| | | JAMB-602 | | |
| EP2287318-0004 | 62.2 | Bacillus sp. | 476 | 296 |
| EP2260105-0445 | 61.5 | B. circulans | 493 | 296 |

### Example 10

### Analysis of Homologous Sequences

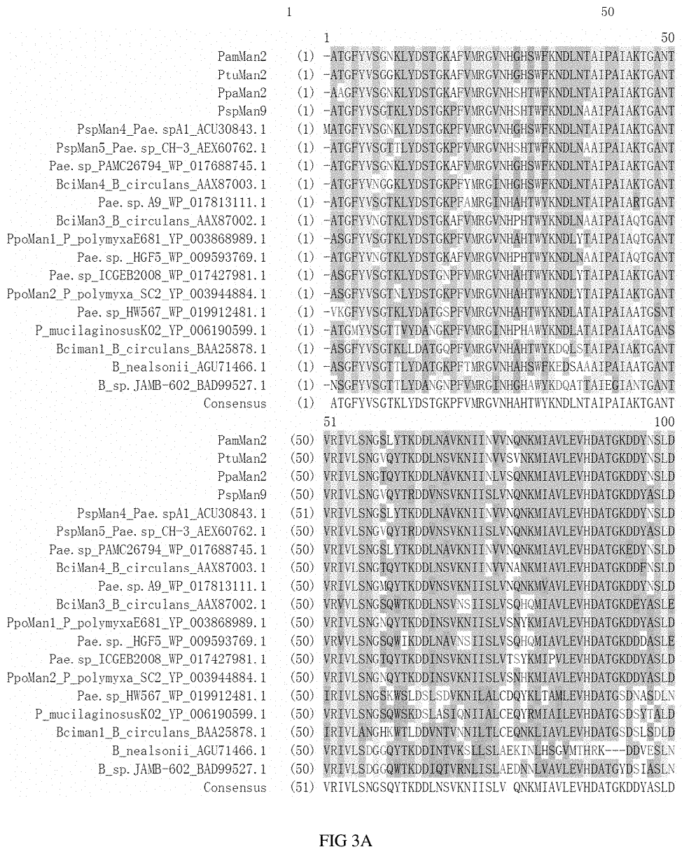

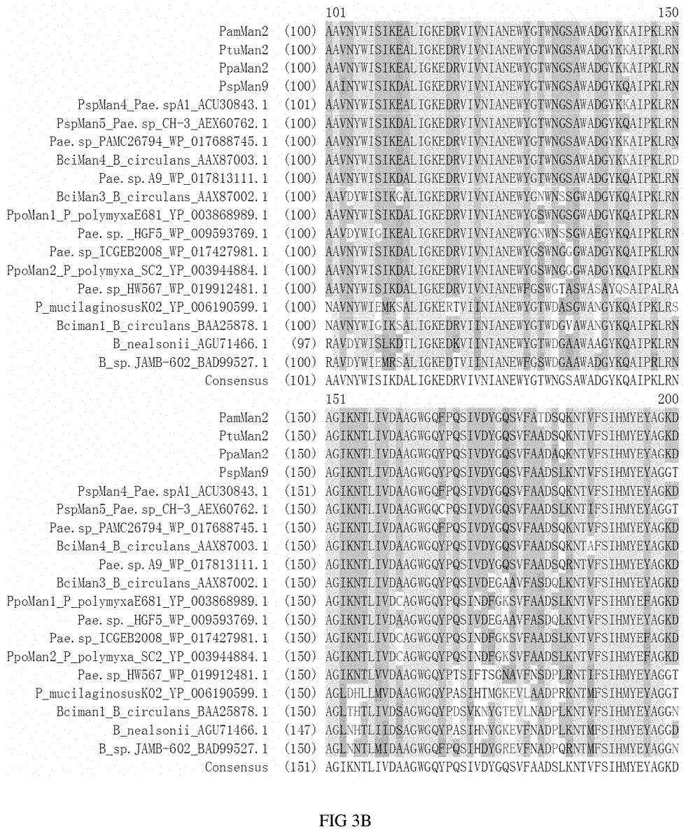

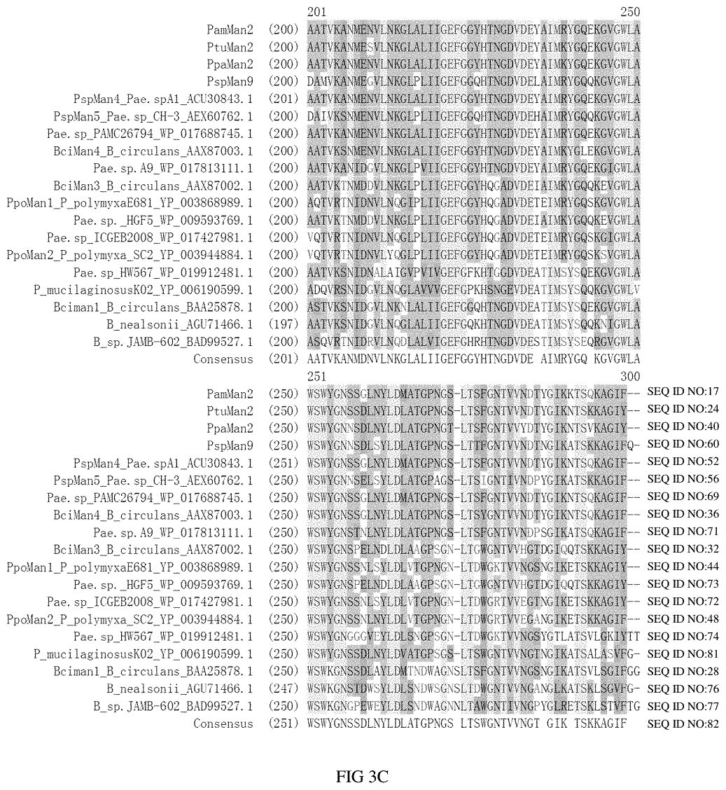

An alignment of the amino acid sequences of the mature BciMan1 (SEQ ID NO:28), BciMan3 (SEQ ID NO:32), BciMan4 (SEQ ID NO:36), PamMan2 (SEQ ID NO:17), PpaMan2 (SEQ ID NO:40), PpoMan1 (SEQ ID NO:44), PpoMan2 (SEQ ID NO:48), PspMan4 (SEQ ID NO:52), PspMan5 (SEQ ID NO:56), PspMan9 (SEQ ID NO:60), and PtuMan2 (SEQ ID NO:24) mannanases with some of the sequences of the mature forms of mannanases from Tables 5A, 6A, and 7A (identified from NCBI searches) is shown in FIG. 3. The full-length, untrimmed sequences were aligned using CLUSTALW software (Thompson et al., Nucleic Acids Research, 22:4673-4680, 1994) with the default parameters, wherein FIG. 3 displays the alignment of amino acids 1-300 and not the alignment of the entire full-length, untrimmed sequences.

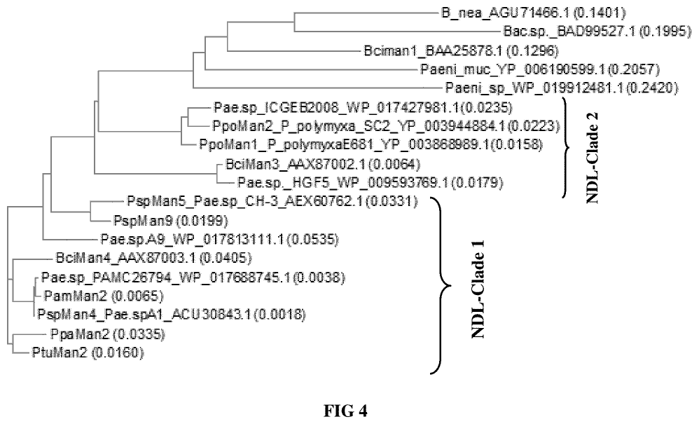

A phylogenetic tree for amino acid sequences of the mannanases aligned in FIG. 3 was built, and is shown on FIG. 4. The full-length, untrimmed sequences were entered in the Vector NTI Advance suite and a Guide Tree was created using the Neighbor Joining (NJ) method (Saitou and Nei, Mol Biol Evol, 4:406-425, 1987). The tree construction was calculated using the following parameters: Kimura's correction for sequence distance and ignoring positions with gaps. AlignX displays the calculated distance values in parenthesis following the molecule name displayed on the tree shown in FIG. 4.

### Example 11

### Unique Features of the NDL-Clade Mannanases

When the mannanases described in Example 10 were aligned common features were shared among BciMan3, BciMan4, PamMan2, PpaMan2, PpoMan1, PpoMan2, PspMan4, PspMan5, PspMan9, and PtuMan2 mannanases. In one case, there is a common pattern of conserved amino acids between residues Trp30 and Ile39, wherein the amino acid positions of the polypeptide are numbered by correspondence with the amino sequence set forth in SEQ ID NO:32. The NDL mannanases share features to create a clade, subsequently termed NDL-Clade, where the term NDL derives from the complete conserved residues NDL near the N-terminus (Asn-Asp-Leu 33-35). The numbering of residues for the mannanases shown is the consecutive linear sequence and are numbered by correspondence with the amino acid sequence set forth in SEQ ID NO:32. The pattern of conserved amino acids related to the NDL-Clade is highlighted in FIG. 5, and can be described as WXₐKNDLXXAI, where Xₐ is F or Y and X is any amino acid; WXₐKNDLX_{b}X_{c}AI, where Xₐ is F or Y, X_{b} is N, Y or A, and X_{c} is A or T; or WF/YKNDLX₁T/AAI, where X₁ is N, Y or A.

The phylogenetic tree described in Example 10 shows a differentiation between the NDL-Clade mannanases and other mannanases. The clade further differentiates into NDL-Clade 1 and NDL-Clade 2 where NDL-Clade 1 includes PtuMan2, PamMan2, PspMan4, BciMan4, PpaMan2, PspMan9 and PspMan5 while NDL-Clade 2 includes BciMan3, PpoMan2 and PpoMan1.

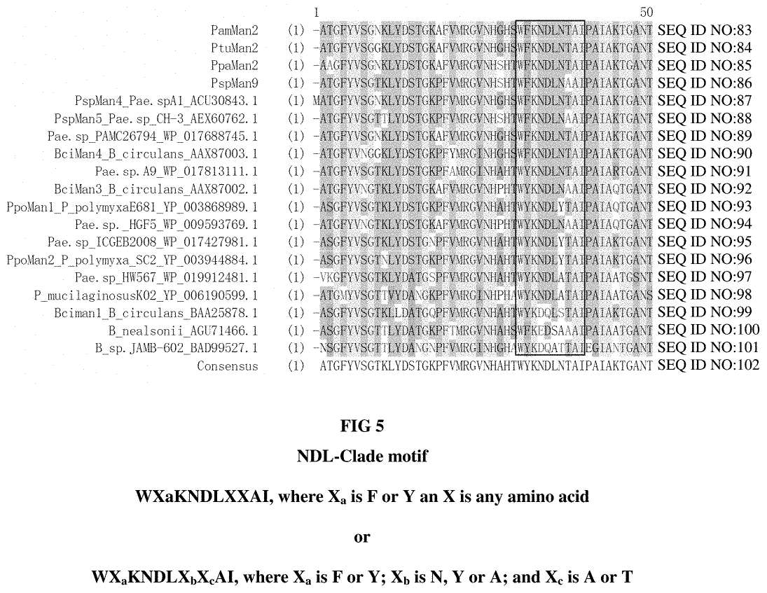

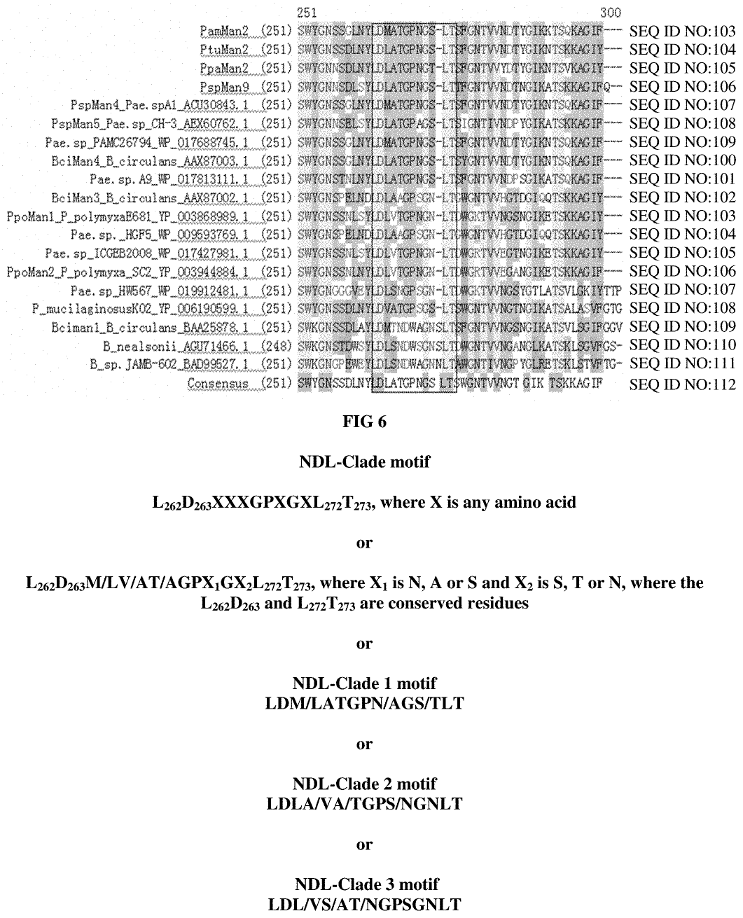

All members of the NDL-Clade have a conserved motif with the key feature of a deletion which is not present in the Bacillus sp. JAMB-602 and other reference mannanase sequences (hereinafter the “deletion motif”). The deletion motif starts at position 262 in the conserved linear sequence of the amino acid sequences set forth in FIG. 6 and includes the sequence LDXXXGPXGXLT, where X is any amino acid or LDM/LV/AT/AGPX₁GX₂LT, where X₁ is N, A or S and X₂ is S, T or N. The sequence further differentiates into LDM/LATGPN/AGS/TLT for NDL-Clade 1 mannanases; LDLA/VA/TGPS/NGNLT for NDL-Clade 2 mannanases; and LDL/VS/AT/NGPSGNLT for NDL-Clade 3 mannanases. All members of the NDL-Clade have a conserved deletion motif not seen in the Bacillus sp. JAMB-602 BAD99527.1, B_nealsonii_AGU71466.1, and Bciman1_B_circulans_BAA25878.1 mannanase sequences. The NDL-Clade deletion motif (i.e., LDM/LV/AT/AGPX₁GX₂LT, where X₁ is N, A or S and X₂ is S, T or N) set forth in FIG. 6 occurs between the conserved residues Leu262-Asp263 (LD) and Leu272-Thr273 (LT).

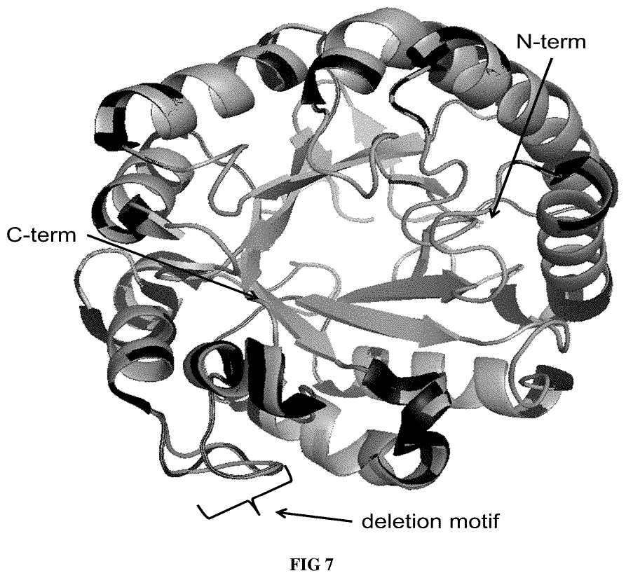

The closest related structure to the NDL-Clade mannanases is that from Bacillus sp. JAMB-602 (1WKY.pdb) and thus this will be used as a reference to understand the probable consequences of the differentiating characteristics of the NDL-Clade mannanases. FIG. 7 shows the structure of Bacillus sp. JAMB-602 (black) and models of the NDL-Clade mannanases PspMan4, PspMan9 and PpaMan2 (gray). The structures of PspMan4, PspMan9 and PpaMan2 were modelled using the “align” option in the Molecular Operating Environment (MOE) software (Chemical Computing Group, Montreal, Quebec, Canada) to look for structural similarities. The alignment applies conserved structural motifs as an additional guide to conventional sequence alignment. This alignment was performed using standard program defaults present in the 2012.10 distribution of MOE. The deletion motif segment is designated with an arrow. This deletion motif is located in a loop in the structure in the C-terminus. The C-terminal region of the Bacillus sp. JAMB-602 mannanase is thought to be important to understanding how these mannanases interact in alkaline environments (Akita et al., Acta Cryst, 60:1490-1492, 2004). It is postulated that the deletion impacts the structure, length and flexibility of this loop which then impacts the activity and performance of the NDL-Clade mannanases.

### Example 12

### Identification of Additional Mannanase from Paenibacillus sp. N021

The entire genome of the Paenibacillus sp. N021strain (DuPont Culture Collection) was sequenced using ILLUMINA® sequencing by synthesis technology. After sequence assembly and annotation, one of the genes identified from this strain, PamMan3, showed homology to members of the NDL-Clade mannanases.

The nucleotide sequence of the PamMan3 gene isolated from Paenibacillus sp. N021 is set forth as SEQ ID NO:61 (the sequence encoding the predicted native signal peptide is shown in bold):

ATGGTCAATCTGAAGAAATGTACGATCTTTACGTTGATTGCTGCGCTC
ATGTTCATGGCTCTGGGGAGTGTTACGCCCAAGGCAGCTGCTGCATCC
GGTTTTTATGTAAGCGGGAATAAGTTATATGACTCGACTGGCAAGCCT
TTTGTCATGAGAGGAATCAATCACGGCCATTCCTGGTTCAAAAATGAT
CTGAATACAGCCATACCTGCTATTGCGAAAACAGGCGCCAACACGGTA
CGAATTGTTCTCTCGAATGGAACACTGTACACCAAAGATGATCTGAAT
TCAGTTAAAAACATAATCAATCTGGTCAATCAGAATAAGATGATCGCC
GTGCTTGAAGTGCATGATGCAACAGGCAAAGACGATTATAACTCGCTG
GATGCAGCCGTGAATTACTGGATCAGCATCAAAGAAGCGTTGATTGGC
AAGGAAGATCGAGTGATCGTTAATATCGCCAACGAATGGTATGGAACC
TGGAACGGCAGCGCTTGGGCAGACGGTTACAAAAAGGCTATTCCGAAG
CTCAGAAACGCAGGCATCAAAAATACGTTGATTGTTGATGCTGCAGGC
TGGGGTCAATATCCACAATCGATTGTCGATTATGGTCAAAGCGTATTC
GCAACAGATACGCTCAAAAATACGGTGTTTTCCATTCATATGTATGAA
TATGCGGGTAAGGATGCGGCAACGGTGAAAGCTAATATGGAGAATGTG
CTGAACAAAGGACTTGCAGTAATCATTGGTGAGTTCGGTGGATATCAC
ACAAATGGTGATGTGGATGAATATGCCATTATGAGATATGGACAAGAG
AAGGGTGTAGGCTGGCTTGCATGGTCATGGTACGGCAACAGTTCCGGT
CTGGGTTATCTGGATCTGGCTACCGGTCCGAACGGAAGTCTCACAAGT
TATGGCAATACGGTAGTTAATGACACATACGGAATCAAAAATACGTCC
CAAAAAGCAGGGATATTTCAATAG.

The amino acid sequence of the PamMan3 precursor protein is set forth as SEQ ID NO:62 (the predicted native signal peptide is shown in bold):

MVNLKKCTIFTLIAALMFMALGSVTPKAAAASGFYVSGNKLYDSTGKP
FVMRGINHGHSWFKNDLNTAIPAIAKTGANTVRIVLSNGTLYTKDDLN
SVKNIINLVNQNKMIAVLEVHDATGKDDYNSLDAAVNYWISIKEALIG
KEDRVIVNIANEWYGTWNGSAWADGYKKAIPKLRNAGIKNTLIVDAAG
WGQYPQSIVDYGQSVFATDTLKNTVFSIHMYEYAGKDAATVKANMENV
LNKGLAVIIGEFGGYHTNGDVDEYAIMRYGQEKGVGWLAWSWYGNSSG
LGYLDLATGPNGSLISYGNTVVNDTYGIKNTSQKAGIFQ.

The sequence of the fully processed mature PamMan3 protein (297 amino acids) is set forth in SEQ ID NO:63:

ASGFYVSGNKLYDSTGKPFVMRGINHGHSWFKNDLNTAIPAIAKTGAN
TVRIVLSNGTLYTKDDLNSVKNIINLVNQNKMIAVLEVHDATGKDDYN
SLDAAVNYWISIKEALIGKEDRVIVNIANEWYGTWNGSAWADGYKKAI
PKLRNAGIKNTLIVDAAGWGQYPQSIVDYGQSVFATDTLKNTVFSIHM
YEYAGKDAATVKANMENVLNKGLAVIIGEFGGYHTNGDVDEYAIMRYG
QEKGVGWLAWSWYGNSSGLGYLDLATGPNGSLTSYGNTVVNDTYGIKN
TSQKAGIFQ.

### Example 13

### Expression of PamMan3

The DNA sequence of the mature form of PamMan3 gene was synthesized and PamMan3 protein was expressed as described in Example 2.

The nucleotide sequence of the synthesized PamMan3 gene in plasmid p2JM-PamMan3 is set forth as SEQ ID NO:64 (the gene has an alternative start codon (GTG), the oligonucleotide encoding the three residue amino-terminal extension (AGK) is shown in bold):

GTGAGAAGCAAAAAATTGTGGATCAGCTTGTTGTTTGCGTTAACGTTA
ATCTTTACGATGGCGTTCAGCAACATGAGCGCGCAGGCTGCTGGAAAA
GCATCAGGCTTTTATGTTTCAGGCAATAAACTTTATGATTCAACAGGA
AAACCGTTTGTTATGAGAGGAATTAATCACGGACATTCATGGTTCAAA
AATGATCTTAACACAGCTATTCCGGCGATTGCGAAGACAGGCGCAAAT
ACAGTTAGAATTGTTCTGTCAAATGGCACGCTGTACACAAAGGACGAT
CTGAACAGCGTTAAAAACATCATTAATCTGGTTAATCAAAATAAGATG
ATTGCAGTTCTGGAAGTCCATGATGCTACAGGCAAAGACGATTACAAT
TCACTGGATGCTGCAGTCAATTACTGGATTTCAATTAAAGAAGCACTG
ATTGGAAAAGAGGACAGAGTTATTGTTAATATCGCAAATGAATGGTAT
GGAACATGGAATGGCAGCGCATGGGCAGATGGCTATAAGAAAGCAATT
CCGAAACTGAGAAACGCAGGCATCAAGAACACGCTTATCGTTGATGCA
GCAGGCTGGGGACAATATCCGCAATCAATTGTTGATTATGGCCAAAGC
GTTTTTGCAACAGACACACTGAAAAACACAGTTTTCTCAATTCATATG
TACGAATATGCCGGAAAGGATGCGGCAACGGTTAAAGCAAATATGGAA
AATGTTCTGAATAAAGGCCTGGCAGTTATTATCGGCGAATTTGGCGGC
TATCATACGAATGGCGATGTTGACGAATACGCGATCATGAGATATGGA
CAGGAGAAAGGCGTTGGCTGGCTTGCGTGGTCATGGTACGGAAATAGC
TCAGGACTGGGCTATCTGGATCTTGCAACGGGACCGAACGGCTCACTT
ACATCATATGGCAACACGGTCGTGAATGATACATACGGCATTAAGAAT
ACATCACAAAAAGCCGGCATTTTTCAA.

The amino acid sequence of the PamMan3 precursor protein expressed from plasmid p2JM-PamMan3 is set forth as SEQ ID NO:65 (the predicted signal sequence is shown in italics, the three residue amino-terminal extension (AGK) is shown in bold):

MRSKKLWISLLFALTLIFTMAFSNMSAQAAGKASGFYVSGNKLYDSTG
KPFVMRGINHGHSWFKNDLNTAIPAIAKTGANTVRIVLSNGTLYTKDD
LNSVKNIINLVNQNKMIAVLEVHDATGKDDYNSLDAAVNYWISIKEAL
IGKEDRVIVNIANEWYGTWNGSAWADGYKKAIPKLRNAGIKNTLIVDA
AGWGQYPQSIVDYGQSVFATDTLKNTVFSIHMYEYAGKDAATVKANME
NVLNKGLAVIIGEFGGYHTNGDVDEYAIMRYGQEKGVGWLAWSWYGNS
SGLGYLDLATGPNGSLTSYGNTVVNDTYGIKNTSQKAGIFQ.

The amino acid sequence of the PamMan3 mature protein expressed from p2JM-PamMan3 plasmid is set forth as SEQ ID NO:66 (the three residue amino-terminal extension (AGK) based on the predicted cleavage site is shown in bold):

AGKASGFYVSGNKLYDSTGKPFVMRGINHGHSWFKNDLNTAIPAIAKT
GANTVRIVLSNGTLYTKDDLNSVKNIINLVNQNKMIAVLEVHDATGKD
DYNSLDAAVNYWISIKEALIGKEDRVIVNIANEWYGTWNGSAWADGYK
KAIPKLRNAGIKNTLIVDAAGWGQYPQSIVDYGQSVFATDTLKNTVFS
IHMYEYAGKDAATVKANMENVLNKGLAVIIGEFGGYHTNGDVDEYAIM
RYGQEKGVGWLAWSWYGNSSGLGYLDLATGPNGSLTSYGNTVVNDTYG
IKNTSQKAGIFQ.

The amino acid sequence of the PamMan3 mature protein, based on the predicted cleavage of the naturally occurring sequence, is set forth as SEQ ID NO:67:

ASGFYVSGNKLYDSTGKPFVMRGINHGHSWFKNDLNTAIPAIAKTGAN
TVRIVLSNGTLYTKDDLNSVKNIINLVNQNKMIAVLEVHDATGKDDYN
SLDAAVNYWISIKEALIGKEDRVIVNIANEWYGTWNGSAWADGYKKAI
PKLRNAGIKNTLIVDAAGWGQYPQSIVDYGQSVFATDTLKNTVFSIHM
YEYAGKDAATVKANMENVLNKGLAVIIGEFGGYHTNGDVDEYAIMRYG
QEKGVGWLAWSWYGNSSGLGYLDLATGPNGSLTSYGNTVVNDTYGIKN
TSQKAGIFQ.

### Example 14

### Purification of PamMan3

PamMan3 was purified via two chromatography steps: hydrophobic interaction chromatography and anion-exchange chromatography. The concentrated and desalted crude protein sample was loaded onto a Phenyl-Sepharose High Performance column pre-equilibrated with 20 mM HEPES (pH 7.4) containing 2.0 M ammonium sulfate. Gradient elution was performed, and fractions with enzymatic activity were pooled and loaded onto a 30 mL Q-Sepharose High Performance column pre-equilibrated with buffer A (20 mM HEPES, pH 7.4). The column was subjected to a gradient elution of 0-50% buffer B (buffer A containing 1 M sodium chloride) in 5 column volumes, followed by 4 column volumes of 100% buffer B. The purity of each fraction was analyzed by SDS-PAGE, and the result showed that the target protein had been effectively purified. The fractions with high purity were pooled and concentrated using an Amicon Ultra-15 device with 10 K MWCO. The final purified protein was stored in 40% glycerol at −20° C. until usage.

### Example 15

### Mannanase Activity of PamMan3

The beta 1-4 mannanase activity of PamMan3 was measured as described in Example 4. The specific activity of purified PamMan3 is summarized in Table 8.

TABLE 8
Specific activities (U/mg) of mannanases at
pH 5.0 and pH 8.2 using different substrates

| | pH 5.0 | | pH 8.2 | |
|---|---|---|---|---|
| | Locust bean | Konjac | Locust bean | Konjac |
| Mannanase | gum | glucomannan | gum | glucomannan |
| PamMan3 | 95 | 167 | 380 | 521 |

### Example 16

### pH Profile of PamMan3

The pH profile of PamMan3 was determined as described in Example 5. The pH optimum and range of ≥70% activity for PamMan3 under these assay conditions is shown in Table 9.

TABLE 9
Optimal pH and pH range of activity for mannanases

| | | pH range |
|---|---|---|
| | Optimum | of ≥70% |
| Mannanase | pH | activity |
| PamMan3 | 7.0 | 6.0-9.0 |

### Example 17

### Temperature Profile of PamMan3

The temperature profile of PamMan3 was determined as described in Example 6. The temperature optimum and temperature range of ≥70% activity for PamMan3 under these assay conditions is shown in Table 10.

TABLE 10
Optimal temperature and temperature
range of activity for mannanases.

| | Optimum | Temperature range |
|---|---|---|
| | Temperature | of ≥70% activity |
| Mannanase | (° C.) | (° C.) |
| PamMan3 | 57 | 47-62 |

### Example 18

### Thermostability of PamMan3

The temperature stability of PamMan3 was determined as described in Example 7. The temperatures at which PamMan3 retain 50% activity (T₅₀) after a 2-hour incubation period under these assay conditions are shown in Table 11.

TABLE 11
Temperature Stability for mannanases.

| | T₅₀ |
|---|---|
| Mannanase | (° C.) |
| PamMan3 | 57 |

### Example 19

### Cleaning Performance of PamMan3

The cleaning performance of PamMan3 was assessed in a high throughput microswatch assay developed to measure galactomannan release from the technical soil. The released reducing sugar was quantified in a PAHBAH (p-Hydroxy benzoic acid hydrazide) assay (Lever, Anal Biochem, 47:248, 1972).

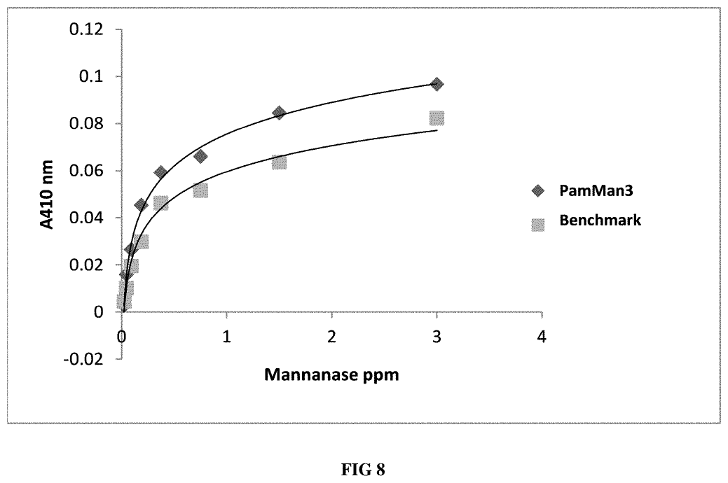

Two 5.5 cm diameter locust bean gum CS-73 (CFT, Vlaardingen, Holland) microswatches were placed into each well of a flat-bottom, non-binding 96-well assay plate. Enzymes were diluted into 50 mM MOPS, pH 7.2, 0.005% Tween-80. Diluted enzyme and microswatch assay buffer (25 mM HEPES, pH 8, 2 mM CaCl₂, 0.005% Tween-80) was added into each well for a combined volume of 100 microliters. Plates were sealed and incubated in an iEMS machine at 25° C. with agitation at 1150 rpm for 30 minutes. 10 microliters reaction mixture was transferred to a PCR plate containing 100 microliters PAHBAH solution each well. Plates were sealed and incubated in a PCR machine at 95° C. for 5 minutes. After the plate was cooled to 4° C., 80 microliters of the supernatant was transferred to a fresh flat-bottom microtiter plate, and the absorbance at 410 nm was measured in a spectrophotometer. FIG. 8 shows the cleaning response of PamMan3 compared to the benchmark (commercially available mannanase, Mannaway®).

### Example 20

### Identification of Homologous Mannanases

The amino acid sequence (297 residues) of the mature form of PamMan3 (SEQ ID NO:67) was subjected to a BLAST search (Altschul et al., Nucleic Acids Res, 25:3389-402, 1997) against the NCBI non-redundant protein database. A similar search was run against the Genome Quest Patent database with search parameters set to default values using SEQ ID NO:67 as the query sequence. Subsets of the search results are shown in Tables 12A and 12B. Percent identity (PID) for both search sets was defined as the number of identical residues divided by the number of aligned residues in the pairwise alignment. The column labeled “Sequence Length” refers to the length (in amino acids) of the protein sequences associated with the listed Accession Nos., while the column labeled “Aligned Length” refers to the length (in amino acids) of the aligned protein sequence used for the PID calculation.

TABLE 12A
List of sequences with percent identity to PamMan3 protein
identified from the NCBI non-redundant protein database

| | PID to | | Sequence | Alignment |
|---|---|---|---|---|
| Accession # | PamMan3 | Organism | Length | Length |
| ACU30843.1 | 95.6 | Paenibacillus sp. A1 | 319 | 296 |
| ETT37549.1 | 95.3 | Paenibacillus sp. FSL R5-192 | 326 | 296 |
| WP_017688745.1 | 94.9 | Paenibacillus sp. PAMC 26794 | 326 | 296 |
| AAX87003.1 | 93.9 | Bacillus circulans | 326 | 296 |
| WP_024633848.1 | 91.9 | Paenibacillus sp. MAEPY1 | 326 | 296 |
| WP_017813111.1 | 89.9 | Paenibacillus sp. A9 | 327 | 296 |
| AEX60762.1 | 87.2 | Paenibacillus sp. CH-3 | 327 | 296 |
| WP_029515900.1 | 81.8 | Paenibacillus sp. WLY78 | 327 | 296 |
| WP_013308634.1/YP_003868989.1 | 81.8 | Paenibacillus polymyxa E681 | 327 | 296 |
| WP_028541088.1 | 81.4 | Paenibacillus sp. UNCCL52 | 327 | 296 |
| WP_023986875.1 | 81.4 | Paenibacillus polymyxa CR1 | 327 | 296 |
| WP_017427981.1 | 81.1 | Paenibacillus sp. ICGEB2008 | 327 | 296 |
| WP_013369280.1/YP_003944884.1 | 80.7 | Paenibacillus polymyxa | 327 | 296 |
| AAX87002.1 | 79.1 | Bacillus circulans | 327 | 296 |
| WP_009593769.1 | 78.0 | Paenibacillus sp. HGF5 | 326 | 296 |
| ETT67091.1 | 77.4 | Paenibacillus sp. FSL H8-457 | 326 | 296 |
| BAA25878.1 | 71.7 | Bacillus circulans | 516 | 297 |
| AIQ62043.1 | 71.4 | Paenibacillus stellifer | 485 | 297 |
| AIQ75360.1 | 70.1 | Paenibacillus odorifer | 573 | 288 |
| ETT49947.1 | 69.8 | Paenibacillus sp. FSL H8-237 | 555 | 288 |

TABLE 12A
List of sequences with percent identity to PamMan3 protein
identified from the NCBI non-redundant protein database

| | PID to | | Sequence | Alignment |
|---|---|---|---|---|
| Accession # | PamMan3 | Organism | Length | Length |
| WP_025708023.1 | 69.2 | Paenibacillus graminis | 294 | 253 |
| WP_028597898.1 | 68.6 | Paenibacillus pasadenensis | 328 | 299 |
| WP_014651264.1/YP_006190599.1 | 68.2 | Paenibacillus mucilaginosus K02 | 475 | 296 |
| WP_013917961.1 | 68.2 | Paenibacillus mucilaginosus KNP414 | 437 | 292 |
| AIQ67798.1 | 67.4 | Paenibacillus graminis | 536 | 288 |
| AGU71466.2 | 65.7 | Bacillus nealsonii | 369 | 297 |
| KGE17399.1 | 65.6 | Paenibacillus wynnii | 516 | 288 |
| WP_017689753.1 | 64.6 | Paenibacillus sp. PAMC 26794 | 595 | 288 |
| WP_027635375.1 | 64.0 | Clostridium butyricum | 470 | 297 |
| WP_028590553.1 | 63.9 | Paenibacillus panacisoli | 596 | 294 |
| WP_031461498.1 | 63.9 | Paenibacillus polymyxa | 796 | 296 |
| WP_006037399.1 | 63.6 | Paenibacillus curdlanolyticus YK9 | 707 | 297 |
| WP_029518464.1 | 62.8 | Paenibacillus sp. WLY78 | 797 | 296 |
| BAD99527.1 | 62.5 | Bacillus sp. JAMB-602 | 490 | 296 |

TABLE 12B
List of sequences with percent identity to PamMan3
protein identified from the Genome Quest database

| | | | Sequence | Alignment |
|---|---|---|---|---|
| Patent ID # | PID | Organism | Length | Length |
| EP2260105-0418 | 93.9 | B. circulans | 326 | 296 |
| CN100410380-0004 | 79.1 | B. circulansB48 | 296 | 296 |
| CN1904052-0003 | 78.4 | B. circulansB48 | 327 | 296 |
| EP2260105-0477 | 71.7 | B. circulans | 516 | 297 |
| WO2014100018-0002 | 68.7 | B. lentus | 299 | 297 |
| US20140199705-0388 | 68.0 | empty | 490 | 297 |
| WO2015022428-0015 | 62.5 | Bacillus sp. | 309 | 296 |
| US20110091941-0001 | 62.5 | Bacillus sp. | 309 | 296 |
| WO2009074685-0001 | 62.5 | Bacillus sp. | 309 | 296 |
| JP2006087401-0001 | 62.5 | Bacillus sp. | 458 | 296 |
| EP2260105-0429 | 62.5 | Bacillus sp. JAMB-602 | 490 | 296 |
| JP2006087401-0003 | 62.5 | Bacillus sp. | 490 | 296 |
| WO2014088940-0002 | 62.3 | B. hemicellulosilyticus | 493 | 297 |
| WO2014124927-0018 | 62.2 | Bacillus sp. I633 | 490 | 296 |
| US20030203466-0008 | 61.62 | B. agaradhaerens | 468 | 297 |

### Example 21

### Analysis of Homologous Mannanase Sequences

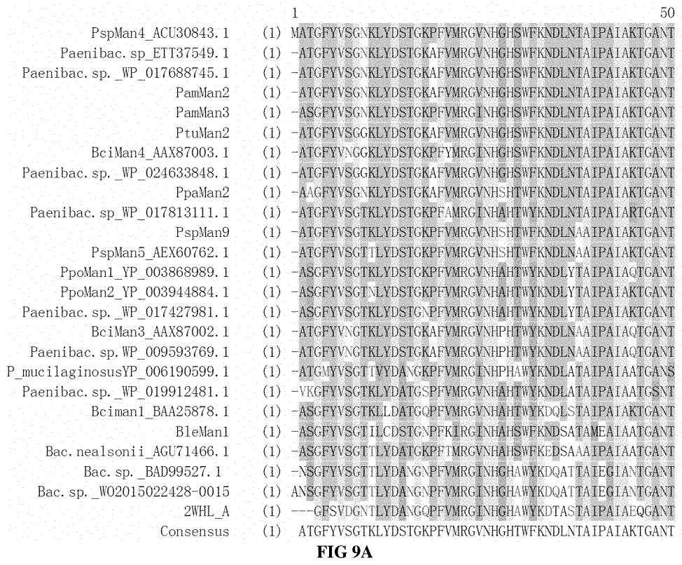

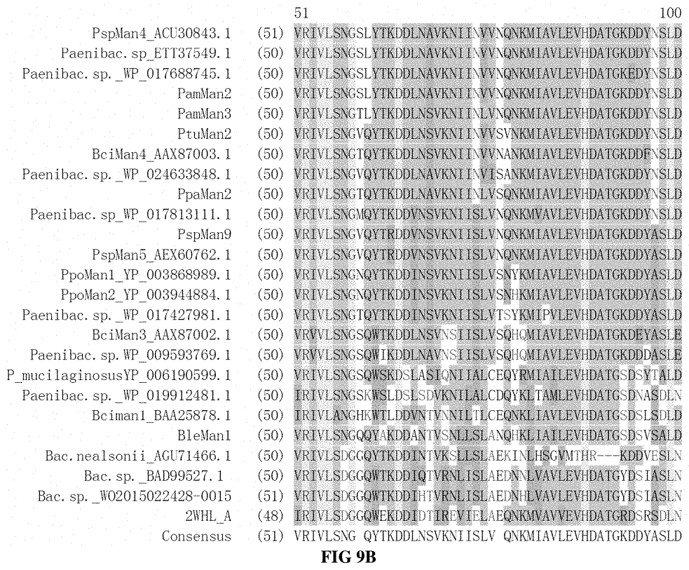

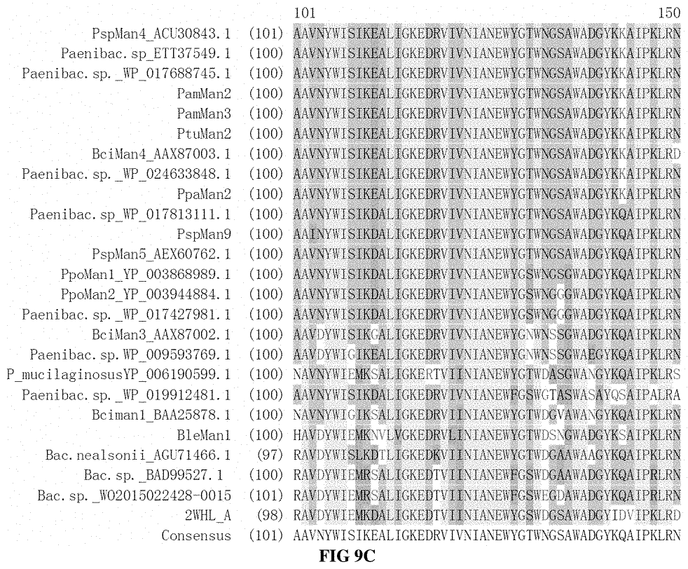

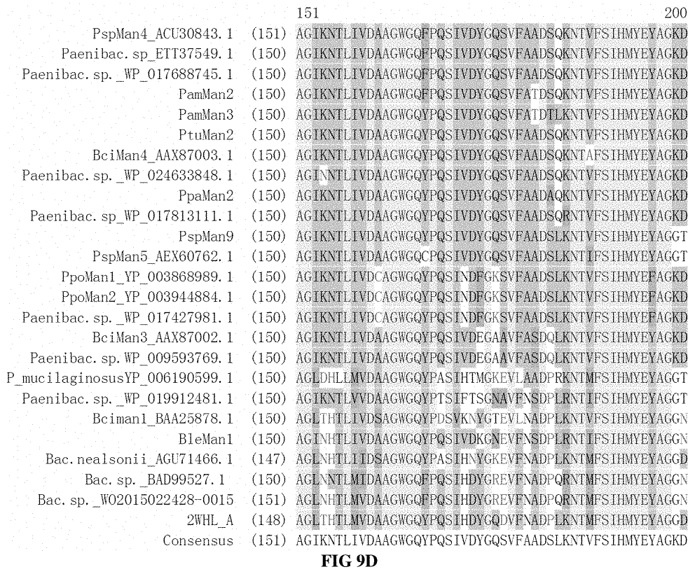

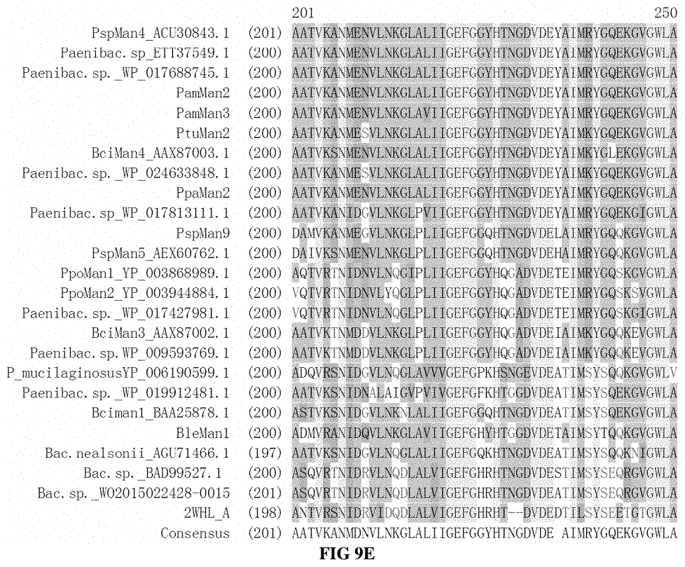

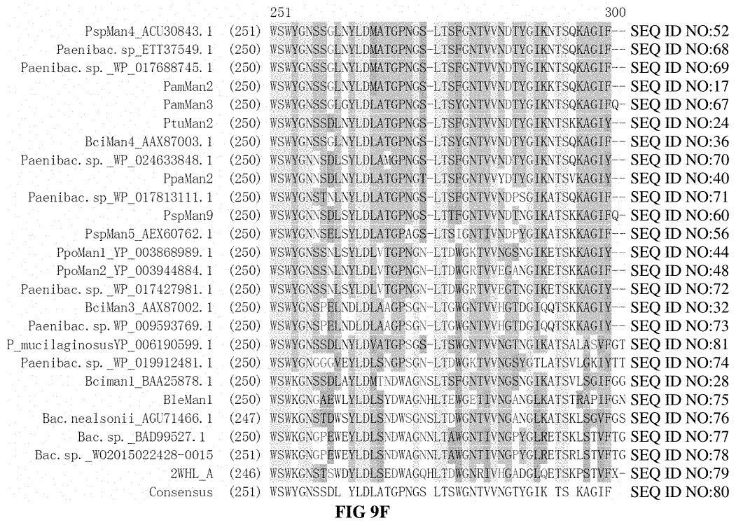

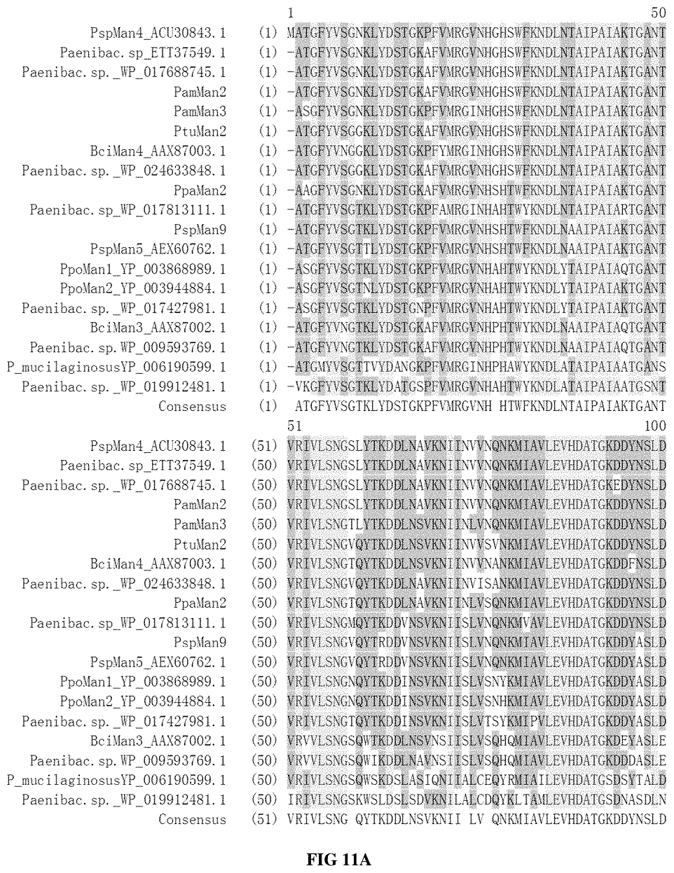

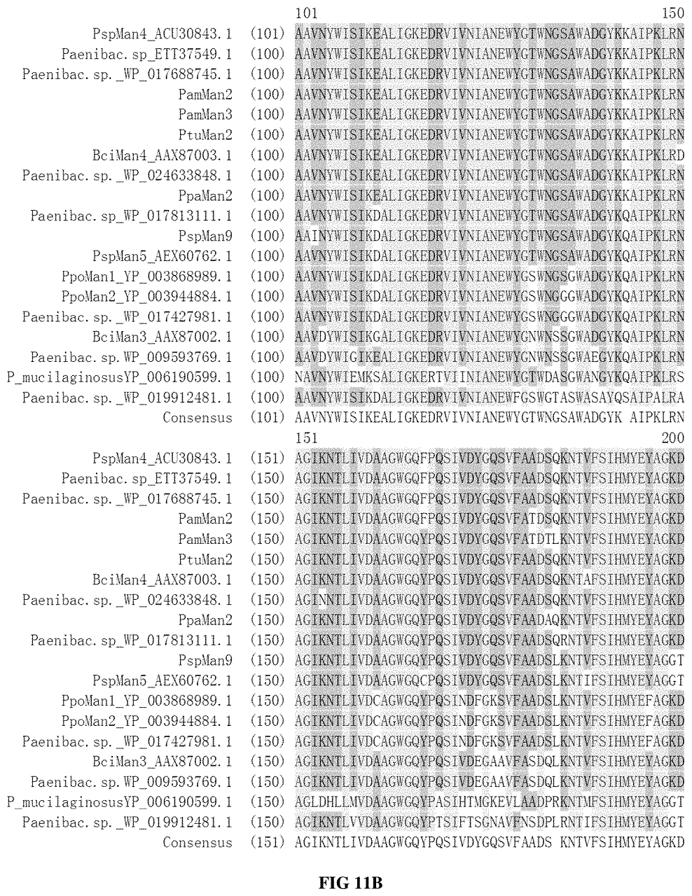

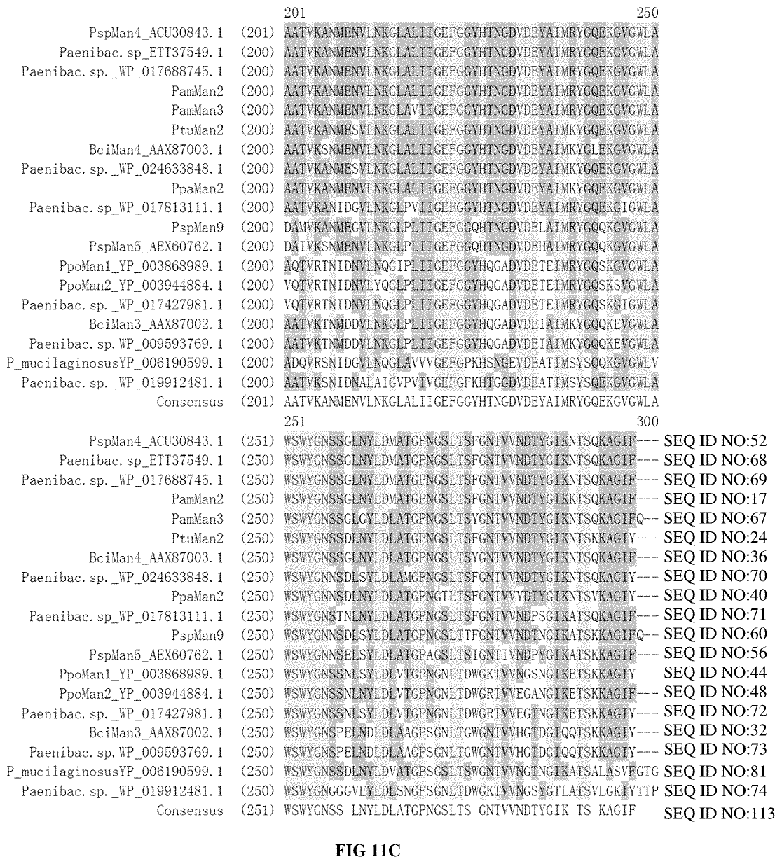

A multiple mannanase amino acid sequence alignment was constructed using the trimmed amino acid sequences set forth in FIG. 5 and the trimmed mature amino acid sequences for: PamMan3 (SEQ ID NO:67), Paenibac.sp_ETT37549.1 (SEQ ID NO:68), Paenibac.sp._WP_024633848.1 (SEQ ID NO:70), BleMan1 (SEQ ID NO:75), Bac.sp_WO2015022428-0015 (SEQ ID NO:78), 2WHL_A (SEQ ID NO:79) and P_mucilaginosus_YP_006190599.1 (SEQ ID NO:81) mannanases, and is shown in FIG. 9. These sequences were aligned using CLUSTALW software (Thompson et al., Nucleic Acids Research, 22:4673-4680, 1994) with the default parameters. Review of the sequence alignment in the region covering the NDL-Clade unique residues (see FIG. 9) shows that mannanases P_mucilaginosus_YP_006190599.1 (SEQ ID NO:81), Paenibac.sp_WP_019912481.1 (SEQ ID NO:74), BciMan3 (SEQ ID NO:32), Paenibac.sp.WP_009593769.1 (SEQ ID NO:73), PpoMan1 (SEQ ID NO:44), PpoMan2 (SEQ ID NO:48), Paenibac.sp_WP_017427981.1 (SEQ ID NO:72), PspMan9 (SEQ ID NO:60), PspMan5 (SEQ ID NO:56), Paenibac.sp. WP_017813111.1 (SEQ ID NO:71), PpaMan2 (SEQ ID NO:40), PtuMan2 (SEQ ID NO:24), Paenibac.sp._WP_024633848.1 (SEQ ID NO:70), PamMan3 (SEQ ID NO:67), BciMan4 (SEQ ID NO:36), PspMan4 (SEQ ID NO:52), PamMan2 (SEQ ID NO:17), Paenibac.sp_ETT37549.1 (SEQ ID NO:68), and Paenibac.sp_WP_017688745.1 (SEQ ID NO:69) all belong to the NDL-Clade, of which a further sequence alignment of the trimmed amino acid sequences was provide using CLUSTALW software (Thompson et al., Nucleic Acids Research, 22:4673-4680, 1994) with the default parameters and is set forth in FIG. 11.

The NDL-Clade can be further differentiated into NDL-Clade 1, NDL-Clade 2, and NDL-Clade 3. NDL-Clade 1 includes PtuMan2, PamMan2, PamMan3, PspMan4, BciMan4, PpaMan2, PspMan9, PspMan5, Paenibac.sp._WP_017813111.1, Paenibac.sp._WP_024633848.1, Paenibac.sp_ETT37549.1, and Paenibac.sp_WP_017688745.1. NDL-Clade 2 includes BciMan3, Paenibac.sp.WP_009593769.1, PpoMan1, PpoMan2, and Paenibac.sp_WP_017427981.1. NDL-Clade 3 includes P_mucilaginosus_YP_006190599.1 and Paenibac.sp_WP_019912481.1.

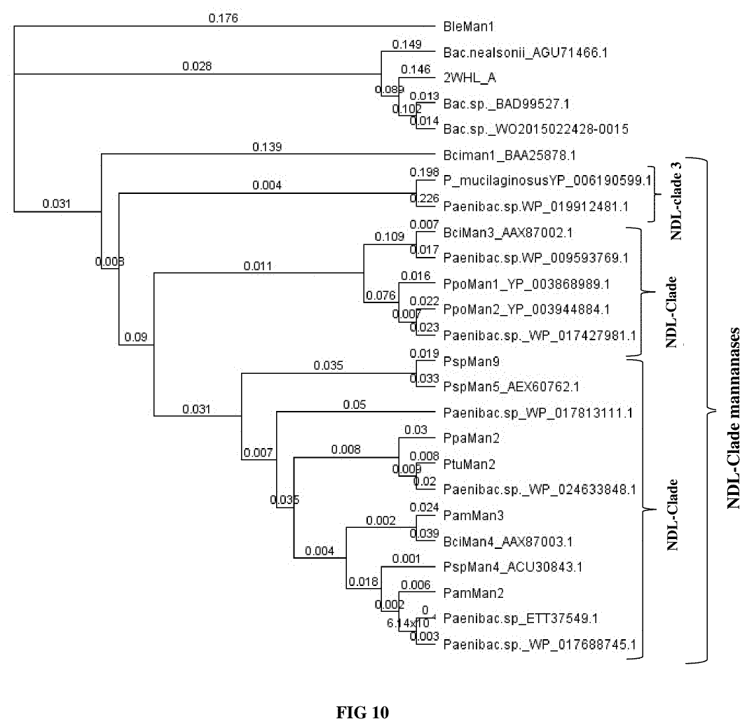

A phylogenetic tree for the trimmed amino acid sequences of the NDL clade mannanases: BciMan1 (SEQ ID NO:28), BciMan3 (SEQ ID NO:32), BciMan4 (SEQ ID NO:36), PamMan2 (SEQ ID NO:17), PpaMan2 (SEQ ID NO:40), PpoMan1 (SEQ ID NO:44), PpoMan2 (SEQ ID NO:48), PspMan4 (SEQ ID NO:52), PspMan5 (SEQ ID NO:56), PspMan9 (SEQ ID NO:60), and PtuMan2 (SEQ ID NO:24), PamMan3 (SEQ ID NO:67), Paenibac.sp_ETT37549.1 (SEQ ID NO:68), Paenibac.sp_WP_017688745.1 (SEQ ID NO:69), Paenibac.sp._WP_024633848.1 (SEQ ID NO:70), Paenibac.sp._WP_017813111.1 (SEQ ID NO:71), Paenibac.sp_WP_017427981.1 (SEQ ID NO:72), Paenibac.sp.WP_009593769.1 (SEQ ID NO:73), Paenibac.sp_WP_019912481.1 (SEQ ID NO:74), BleMan1 (SEQ ID NO:75), Bac.nealsonii_AGU71466.1 (SEQ ID NO:76), Bac.sp._BAD99527.1 (SEQ ID NO:77), Bac.sp_WO2015022428-0015 (SEQ ID NO:78), and 2WHL_A (SEQ ID NO:79) and P_mucilaginosus_YP_006190599.1 (SEQ ID NO:81), was built, and shown on FIG. 10. The trimmed sequences were entered in the Vector NTI Advance suite and the alignment file was subsequently imported into The Geneious Tree Builder program (Geneious 8.1.2) and the phylogenetic tree shown in FIG. 10 was built using the The Geneious Tree Builder, Neighbor-Joining tree build method. The percent sequences identity among these sequences was calculated and is shown on Table 13.

TABLE 13
The percent sequence identity among NDL-1 clade mannanase mature sequences.

| | | | Paenibac.sp._{—} | | | |
|---|---|---|---|---|---|---|
| | PspMan4_ACU30843.1 | Paenibac.sp_ETT37549.1 | WP_017688745.1 | PtuMan2 | PpaMan2 | PamMan2 |
| PspMan4_ACU30843.1 | | 99.7 | 99.3 | 95.3 | 93.9 | 99 |
| Paenibac.sp_ETT37549.1 | 99.7 | | 99.7 | 95.6 | 94.3 | 99.3 |
| Paenibac.sp._{—} | 99.3 | 99.7 | | 95.3 | 93.9 | 99 |
| WP_017688745.1 | | | | | | |
| PtuMan2 | 95.3 | 95.6 | 95.3 | | 95.3 | 94.9 |
| PpaMan2 | 93.9 | 94.3 | 93.9 | 95.3 | | 93.6 |
| PamMan2 | 99 | 99.3 | 99 | 94.9 | 93.6 | |
| PamMan3 | 95.6 | 95.3 | 94.9 | 93.2 | 92.9 | 95.3 |
| BciMan4_AAX87003.1 | 94.3 | 93.9 | 93.6 | 94.3 | 91.6 | 93.2 |
| Paenibac.sp._WP_{—} | 94.3 | 94.6 | 94.3 | 97.3 | 94.6 | 93.9 |
| 024633848.1 | | | | | | |
| Paenibac.sp_{—} | 89.9 | 89.5 | 89.2 | 89.2 | 88.2 | 89.2 |
| WP_017813111.1 | | | | | | |
| PspMan9 | 88.5 | 88.2 | 87.8 | 89.2 | 88.5 | 87.8 |
| PspMan5_AEX60762.1 | 87.5 | 87.2 | 86.8 | 87.2 | 86.8 | 86.8 |

| | | | Paenibac.sp._{—} | Paenibac.sp_{—} | | |
|---|---|---|---|---|---|---|
| | PamMan3 | BciMan4_AAX87003.1 | WP_024633848.1 | WP_017813111.1 | PspMan9 | PspMan5_AEX60762.1 |
| PspMan4_ACU30843.1 | 95.6 | 94.3 | 94.3 | 89.9 | 88.5 | 87.5 |
| Paenibac.sp_ETT37549.1 | 95.3 | 93.9 | 94.6 | 89.5 | 88.2 | 87.2 |
| Paenibac.sp._{—} | 94.9 | 93.6 | 94.3 | 89.2 | 87.8 | 86.8 |
| WP_017688745.1 | | | | | | |
| PtuMan2 | 93.2 | 94.3 | 97.3 | 89.2 | 89.2 | 87.2 |
| PpaMan2 | 92.9 | 91.6 | 94.6 | 88.2 | 88.5 | 86.8 |
| PamMan2 | 95.3 | 93.2 | 93.9 | 89.2 | 87.8 | 86.8 |
| PamMan3 | | 93.9 | 91.9 | 89.9 | 88.2 | 87.2 |
| BciMan4_AAX87003.1 | 93.9 | | 92.9 | 88.5 | 86.1 | 86.1 |
| Paenibac.sp._WP_{—} | 91.9 | 92.9 | | 87.5 | 88.2 | 86.1 |
| 024633848.1 | | | | | | |
| Paenibac.sp_{—} | 89.9 | 88.5 | 87.5 | | 89.2 | 87.5 |
| WP_017813111.1 | | | | | | |
| PspMan9 | 88.2 | 86.1 | 88.2 | 89.2 | | 94.9 |
| PspMan5_AEX60762.1 | 87.2 | 86.1 | 86.1 | 87.5 | 94.9 | |

We claim:

1. A polypeptide or active fragment thereof in the NDL-Clade.

2. The polypeptide or active fragment thereof of claim 1, wherein said polypeptide further comprises an amino acid sequence having at least 70% identity to an amino acid sequence selected from SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81.

3. The polypeptide or active fragment thereof of any preceding claim, wherein said polypeptide is a recombinant polypeptide.

4. The polypeptide or active fragment thereof of any preceding claim, wherein the polypeptide or active fragment thereof is an endo-β-mannanase.

5. The polypeptide or active fragment thereof of any preceding claim, wherein the polypeptide or active fragment thereof contains Asn33-Asp-34-Leu35, wherein the amino acid positions of the polypeptide are numbered by correspondence with the amino sequence set forth in SEQ ID NO:32 and are based on the conserved linear sequence numbering.

6. The polypeptide or an active fragment thereof of any preceding claim, wherein the polypeptide further comprises a WXₐKNDLXXAI motif at positions 30-38, wherein Xₐ is F or Y and X is any amino acid, wherein the amino acid positions of the polypeptide are numbered by correspondence with the amino sequence set forth in SEQ ID NO:32 and are based on the conserved linear sequence numbering.

7. The polypeptide or an active fragment thereof of any preceding claim, wherein the polypeptide further comprises a WXₐKNDLX_{b}X_{c}AI motif at positions 30-38, wherein Xₐ is F or Y, X_{b} is N, Y or A, and X_{c} is A or T, wherein the amino acid positions of the polypeptide are numbered by correspondence with the amino sequence set forth in SEQ ID NO:32 and are based on the conserved linear sequence numbering.

8. The polypeptide or an active fragment thereof of any preceding claim, wherein the NDL-Clade polypeptide further comprises a L₂₆₂D₂₆₃XXXGPXGXL₂₇₂T₂₇₃, motif at positions 262-273, where X is any amino acid and wherein the amino acid positions of the polypeptide are numbered by correspondence with the amino sequence set forth in SEQ ID NO:32 and are based on the conserved linear sequence numbering.

9. The polypeptide or an active fragment thereof of any preceding claim, wherein the NDL-Clade polypeptide further comprises a L₂₆₂D₂₆₃M/LV/AT/AGPX₁GX₂L₂₇₂T₂₇₃ motif at positions 262-273, where X₁ is N, A or S and X₂ is S, T or N, and wherein the amino acid positions of the polypeptide are numbered by correspondence with the amino sequence set forth in SEQ ID NO:32 and are based on the conserved linear sequence numbering.

10. The polypeptide or active fragment thereof of any preceding claim, wherein the NDL-Clade polypeptide is an NDL-Clade-1 polypeptide further comprising a LDM/LATGPA/NGS/TLT motif at positions 262-273, wherein the amino acid positions of the polypeptide are numbered by correspondence with the amino sequence set forth in SEQ ID NO:32 and are based on the conserved linear sequence numbering.

11. The polypeptide or active fragment thereof of any preceding claim, wherein the NDL-Clade polypeptide is an NDL-Clade 2 polypeptide further comprising a LDLA/VA/TGPS/NGNLT motif at positions 262-273, wherein the amino acid positions of the polypeptide are numbered by correspondence with the amino sequence set forth in SEQ ID NO:32 and are based on the conserved linear sequence numbering.

12. The polypeptide or an active fragment thereof of any preceding claim, wherein the NDL-Clade polypeptide is and NDL-Clade 3 polypeptide comprising a LDM/LATGPA/NGS/TLT motif at positions 262-273, wherein the amino acid positions of the polypeptide are numbered by correspondence with the amino sequence set forth in SEQ ID NO:32 and are based on the conserved linear sequence numbering.

13. The polypeptide or an active fragment thereof of any preceding claim, wherein the polypeptide has mannanase activity.

14. The polypeptide or an active fragment thereof of any preceding claim, wherein the mannanase activity is activity on locust bean gum galactomannan.

15. The polypeptide or an active fragment thereof of any preceding claim, wherein the mannanase activity is activity on konjac glucomannan.

16. The polypeptide or an active fragment thereof of any preceding claim, wherein the mannanase activity is in the presence of a surfactant.

17. The polypeptide or an active fragment thereof of any preceding claim, wherein the polypeptide retains at least 70% of its maximal mannanase activity at a pH range of 4.5-9.0.

18. The polypeptide or an active fragment thereof of any preceding claim, wherein the polypeptide retains at least 70% of its maximal mannanase activity at a pH range of 5.5-8.5.

19. The polypeptide or an active fragment thereof of any preceding claim, wherein the polypeptide retains at least 70% of its maximal mannanase activity at a pH range of 6.0-7.5.

20. The polypeptide or an active fragment thereof of any preceding claim, wherein the polypeptide retains at least 70% of its maximal mannanase activity at a temperature range of 40° C. to 70° C.

21. The polypeptide or an active fragment thereof of any preceding claim, wherein the polypeptide retains at least 70% of its maximal mannanase activity at a temperature range of 45° C. to 65° C.

22. The polypeptide or an active fragment thereof of any preceding claim, wherein the polypeptide retains at least 70% of its maximal mannanase activity at a temperature range of 50° C. to 60° C.

23. The polypeptide or an active fragment thereof of any preceding claim, wherein the polypeptide has cleaning activity in a detergent composition.

24. The polypeptide or an active fragment thereof of any preceding claim, wherein the polypeptide has mannanase activity in the presence of a protease.

25. The polypeptide or an active fragment thereof of any preceding claim, wherein the polypeptide is capable of hydrolyzing a substrate selected from the group consisting of guar gum, locust bean gum, and combinations thereof.

26. The polypeptide or an active fragment thereof of any preceding claim, wherein the polypeptide does not further comprise a carbohydrate-binding module.

27. A cleaning composition comprising the polypeptide of any one of claims 1-26.

28. A cleaning composition comprising an amino acid sequence having at least 70% identity to an amino acid sequence selected from SEQ ID NO: 2, 4, 6, 8, 10, 12, 14, 16, 17, 19, 21, 23, 24, 26, 27, 28, 30, 31, 32, 34, 35, 36, 38, 39, 40, 42, 43, 44, 46, 47, 48, 50, 51, 52, 54, 55, 56, 58, 59, 60, 62, 63, 65, 66, 67, 68, 69, 70, 71, 72, 73, 74, and 81.

29. The cleaning composition of claim 27 or 28, further comprising a surfactant.

30. The cleaning composition of claim 29, wherein the surfactant is an ionic surfactant.

31. The cleaning composition of claim 30, wherein the ionic surfactant is selected from the group consisting of an anionic surfactant, a cationic surfactant, a zwitterionic surfactant, and a combination thereof.

32. The cleaning composition of any one of claims 27-31, further comprising an enzyme selected from the group consisting of acyl transferases, amylases, alpha-amylases, beta-amylases, alpha-galactosidases, arabinases, arabinosidases, aryl esterases, beta-galactosidases, beta-glucanases, carrageenases, catalases, cellobiohydrolases, cellulases, chondroitinases, cutinases, endo-beta-1, 4-glucanases, endo-beta-mannanases, exo-beta-mannanases, esterases, exo-mannanases, galactanases, glucoamylases, hemicellulases, hyaluronidases, keratinases, laccases, lactases, ligninases, lipases, lipolytic enzymes, lipoxygenases, mannanases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, and combinations thereof.

33. The cleaning composition of any one of claims 27-32, wherein the cleaning composition is a detergent composition selected from the group consisting of a laundry detergent, a fabric softening detergent, a dishwashing detergent, and a hard-surface cleaning detergent.

34. The cleaning composition of any one of claims 27-33, wherein the cleaning composition is in a form selected from the group consisting of a liquid, a powder, a granulated solid, a tablet, a sheet, and a unit dose.

35. The cleaning composition of any one of claims 27-34, wherein said composition is phosphate-free.

36. The cleaning composition of any one of claims 27-34, wherein said composition contains phosphate.

37. The cleaning composition of any one of claims 27-34, wherein said composition is boron-free.

38. The cleaning composition of any one of claims 27-34, wherein said composition contains boron.

39. The cleaning composition of any one of claims 27-34, further comprising at least one adjunct ingredient.

40. A method for hydrolyzing a mannan substrate present in a soil or stain on a surface, comprising: contacting the surface with the cleaning composition of any one of claims 27-39 to produce a clean surface.

41. A method of textile cleaning comprising: contacting a soiled textile with the cleaning composition of any one of claims 27-39 to produce a clean textile.

42. An nucleic acid encoding the recombinant polypeptide of any one of claims 1-26.

43. The nucleic acid of claim 42, wherein said nucleic acid is isolated.

44. An expression vector comprising the nucleic acid of claim 42 or 43 operably linked to a regulatory sequence.

45. A host cell comprising the expression vector of claim 44.

46. The host cell of claim 45, wherein the host cell is a bacterial cell or a fungal cell.

47. A method of producing an endo-β-mannanase, comprising: culturing the host cell of claim 45 or 46 in a culture medium, under suitable conditions to produce a culture comprising the endo-β-mannanase.

48. The method of claim 47, further comprising removing the host cells from the culture by centrifugation, and removing debris of less than 10 kDa by filtration to produce an endo-β-mannanase-enriched supernatant.

49. A method for hydrolyzing a polysaccharide, comprising: contacting a polysaccharide comprising mannose with the supernatant of claim 48 to produce oligosaccharides comprising mannose.

50. The method of claim 49, wherein the polysaccharide is selected from the group consisting of mannan, glucomannan, galactomannan, galactoglucomannan, and combinations thereof.

51. A food or feed composition and/or food additive comprising the polypeptide of any of claims 1-26.

52. A method for preparing a food or feed composition and/or food or feed additive, comprising mixing the polypeptide of any of claims 1-26 with one or more food or feed and/or food or feed additive ingredients.

53. Use of the polypeptide according to any of claims 1-26 in the preparation of a food or feed composition and/or food or feed additive and/or food or feed stuff and/or pet food.

54. The food or feed composition of claim 51, wherein the food or feed composition is a fermented beverage such as beer.

55. The method of claim 52, wherein the food or feed composition is a fermented beverage such as beer and wherein the one or more food ingredients comprise malt or adjunct.

56. Use of the polypeptide according to any of claims 1-26 in the production of a fermented beverage, such as a beer.

57. A method of providing a fermented beverage comprising the step of contacting a mash and/or a wort with a polypeptide according to any of claims 1-26.

58. A method of providing a fermented beverage comprising the steps of:
a) preparing a mash,
b) filtering the mash to obtain a wort, and
c) fermenting the wort to obtain a fermented beverage, such as a beer wherein a polypeptide according to any of claims 1-26 is added to:
i. the mash of step (a) and/or
ii. the wort of step (b) and/or
iii. the wort of step (c).

59. A fermented beverage, such as a beer, produced by a method according to claim 57 or 58.

60. Use according to claim 56, method according to claim 57 or 58, or fermented beverage according to claim 59, wherein the fermented beverage is a beer, such as full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavoured malt beverages, e. g., citrus flavoured, such as lemon-, orange-, lime-, or berry-flavoured malt beverages, liquor flavoured malt beverages, e.g., vodka-, rum-, or tequila-flavoured malt liquor, or coffee flavoured malt beverages, such as caffeine-flavoured malt liquor, and the like.
